(19) United States
(12) Patent Application Publication
(10) Pub. No.: US 2024/0401057 A1
(43) Pub. Date: Dec. 5, 2024

(54) ADAMTS14 INHIBITION

(71) Applicants: Boehringer Ingelheim International GmbH, Ingelheim Am Rhein (DE); The General Hospital Corporation d/b/a Massachusetts General Hospital, Boston, MA (US)

(72) Inventors: Franziska Elena Herrmann, Ingelheim Am Rhein (DE); Quang Huy Le, Ingelheim Am Rhein (DE); Peter Seither, Ingelheim Am Rhein (DE); Benjamin D. Medoff, Boston, MA (US); Daniela Vanessa Moutinho Dos Santos, Boston, MA (US); Yang Yang, Boston, MA (US)

(73) Assignees: Boehringer Ingelheim International GmbH, Ingelheim Am Rhein (DE); The General Hospital Corporation d/b/a Massachusetts General Hospital, Boston, MA (US)

(21) Appl. No.: 18/698,973

(22) PCT Filed: Oct. 7, 2022
(86) PCT No.: PCT/EP2022/077976

§ 371 Date: Apr. 5, 2024

(30) Foreign Application Priority Data
Oct. 8, 2021 (EP) 21201765.1
Apr. 13, 2022 (EP) 22168132.3

(51) Int. Cl. C12N 15/113; A61P 43/00

(52) U.S. Cl. CPC C12N 15/1137; A61P 43/00; C12N 2310/14; C12Y 304/24

(57) ABSTRACT

Methods of treating and preventing fibrosis, and diseases/disorders characterised by fibrosis, through ADAMTS14 inhibition are disclosed, as well as agents for use in such methods.

This application claims priority from EP21201765.1 filed 8 Oct. 2021 and EP22168132.3 filed 13 Apr. 2022, the contents and elements of which are herein incorporated by reference for all purposes.

### TECHNICAL FIELD

The present disclosure relates to the fields of treatment and prophylaxis of disease, particularly through inhibition of ADAMTS14.

### BACKGROUND

Idiopathic Pulmonary Fibrosis (IPF) is a progressive lung disease with short life-expectancy from the time of diagnosis [1]. IPF causes stiffening of the interstitial tissue and decreased gas exchange. Increased stiffness promotes activation of YAP (Yes-associated Protein) and TAZ (Transcription co-activator with PDZ-binding motif), two transcription co-activators that are activated by mechanical cues in the environment [4-6]. In response to high stiffness, YAP and TAZ translocate to the nucleus of the lung fibroblasts, turning on pro-fibrotic genes. Consistent with this, YAP and TAZ are enriched in the nuclei of fibroblasts in fibrotic areas of the IPF lung [7]. YAP and TAZ are also effectors of the TGFβ signaling pathway and are required for fibroblast activation in the presence of TGFβ [8]. YAP and TAZ activity are kept in check by the Hippo pathway kinases through phosphorylation on multiple serine residues [9]. Transferring fibroblasts overexpressing a non-phosphorylatable form of YAP and TAZ into mouse lungs confers them with fibrogenic capability, and the mice developed lung fibrosis [7]. In summary, YAP and TAZ are important potential targets for IPF treatments.

ADAMTS14 (a disintegrin and metalloproteinase with thrombospondin motif 14) is an extracellular enzyme with N-terminal pro-collagen peptidase activity. It is structurally similar to ADAMTS2 and ADAMTS3 and may have redundant enzyme activity with ADAMTS2 [10]. Single nucleotide polymorphism (SNP) analysis suggests a role for ADAMTS14 in multiple sclerosis (MS), Achille tendon, osteoarthritis, and cancer [16-19]. A potential substrate repertoire of ADAMTS14 was proposed by Beckhouche et al. FASEB J. (2016) 30(5):1741-56, including substrates implicated in tissue maintenance, remodelling, wound repair and immune cell function. However, the putative ADAMTS14 substrates have pleiotropic roles in various physiological functions, and so it is not clear from this study whether and what kind of role ADAMTS14 plays in human disease.

As noted above, ADAMTS14 has been reported to possess aminoprocollagen type I collagenase activity, and e.g. Shiomi et al., Pathol Int. (2010) 60(7): 477-496 describes ADAMTS14 as being an extracellular matrix-degrading proteinase. WO 03/042379 A2 describes the identification of ADAMTS14 (see e.g. para [001]). US 2002/0119555 A1 identifies a protein having the same sequence as ADAMTS14 designated ‘53014’ as a member of the ADAMTS metalloprotease family, and teaches the use of this protein as a therapeutic agent for the treatment of diseases associated with undesirable extracellular matrix accumulation, e.g. disorders characterised by fibrosis (see e.g. paras [0018], [0067]-[0068], [0350]). Similarly, US 2016/0331817 A1 describes ADAMTS14 as an anti-fibrotic agent (see e.g. para [0012]), and teaches the administration of ADAMTS14 to subjects for the treatment of fibrotic disease (see e.g. claim 2).

### SUMMARY

In a first aspect, the present disclosure provides an ADAMTS14 inhibitor for use in the treatment or prevention of fibrosis.

Also provided is the use of an ADAMTS14 inhibitor in the manufacture of a medicament for treating or preventing fibrosis.

Also provided is a method of treating or preventing fibrosis in a subject, comprising administering to a subject a therapeutically- or prophylactically-effective amount of an ADAMTS14 inhibitor.

In some embodiments, the fibrosis is fibrosis of: an organ or tissue of the respiratory system, lung, bronchioles, alveoli, airways, nasal cavity, oral cavity, pharynx, larynx, trachea, bronchus, an organ or tissue of the cardiovascular system, heart, blood vessels, an organ or tissue of the gastrointestinal system, liver, bowel, small intestine, large intestine, colon, pancreas, skin, eye, an organ or tissue of the nervous system, brain, an organ or tissue of the urogenital system, kidney, ovaries, fallopian tubes, an organ or tissue of the musculoskeletal system, muscle tissue, or bone marrow.

In some embodiments, the fibrosis is fibrosis of a disease or condition selected from: pulmonary fibrosis, interstitial lung disease (ILD), idiopathic interstitial pneumonia (IIP), idiopathic pulmonary fibrosis (IPF), cystic fibrosis, progressive massive fibrosis, scleroderma, obliterative bronchiolitis, Hermansky-Pudlak syndrome, asbestosis, silicosis, sarcoidosis, tumor stroma in lung disease, chronic obstructive pulmonary disease (COPD), emphysema, chronic bronchitis, asthma, chronic liver disease, liver fibrosis, cirrhosis, non-alcoholic fatty liver disease (NAFLD), steatohepatitis, non-alcoholic steatohepatitis (NASH), alcoholic liver disease (ALD), alcoholic fatty liver (AFL), alcoholic hepatitis, alcoholic steatohepatitis (ASH), primary biliary cirrhosis (PBC), schistosomal liver disease, hepatocellular carcinoma (HCC), hypertrophic cardiomyopathy (HCM), dilated cardiomyopathy (DCM), fibrosis of the atrium, atrial fibrillation, fibrosis of the ventricle, ventricular fibrillation, myocardial fibrosis, Brugada syndrome, myocarditis, endomyocardial fibrosis, myocardial infarction, fibrotic vascular disease, hypertension, hypertensive heart disease, arrhythmogenic right ventricular cardiomyopathy (ARVC), atherosclerosis, chronic pulmonary hypertension, AIDS-associated pulmonary hypertension, varicose veins, cerebral infarcts, tubulointerstitial fibrosis, glomerular fibrosis, renal fibrosis, nephritic syndrome, Alport's syndrome, HIV-associated nephropathy, polycystic kidney disease, Fabry's disease, diabetic nephropathy, chronic glomerulonephritis, nephritis associated with systemic lupus, pancreatic fibrosis, chronic pancreatitis, endometriosis, gliosis, Alzheimer's disease, multiple sclerosis, muscular dystrophy, Duchenne muscular dystrophy (DMD), Becker's muscular dystrophy (BMD), fibrotic myopathy, inflammatory bowel disease (IBD), Crohn's disease, microscopic colitis, primary sclerosing cholangitis (PSC), scleroderma, nephrogenic systemic fibrosis, Dupuytren's contracture, cutis keloid, Grave's opthalmopathy, epiretinal fibrosis, retinal fibrosis, subretinal fibrosis, subretinal fibrosis associated with macular degeneration, wet age-related macular degeneration (AMD), diabetic retinopathy, glaucoma, corneal fibrosis, post-surgical fibrosis, post-surgical fibrosis of the posterior capsule following cataract surgery, post-surgical fibrosis of the bleb following trabeculectomy for glaucoma, conjunctival fibrosis, subconjunctival fibrosis, arthrofibrosis, arthritis, adhesive capsulitis, progressive systemic sclerosis (PSS), chronic graft versus host disease (GVHD), fibrotic pre-neoplastic disease, fibrotic neoplastic disease, fibrosis induced by chemical insult, or fibrosis induced by environmental insult, fibrosis induced by cancer chemotherapy, fibrosis induced by pesticides, fibrosis induced by radiation, fibrosis induced by cancer radiotherapy, cancer, hepatocellular carcinoma, gastric cancer, esophageal cancer, head and neck cancer, colorectal cancer, pancreatic cancer, cervical cancer, vulvar cancer, mediastinal fibrosis, retroperitoneal fibrosis, myelofibrosis and Peyronie's disease.

In some embodiments, the fibrosis is fibrosis of: an organ or tissue of the respiratory system, lung, bronchioles, alveoli, airways, nasal cavity, oral cavity, pharynx, larynx, trachea or bronchus.

In some embodiments, the fibrosis is fibrosis of a disease or condition selected from: pulmonary fibrosis, interstitial lung disease (ILD), idiopathic interstitial pneumonia (IIP), idiopathic pulmonary fibrosis (IPF), cystic fibrosis, progressive massive fibrosis, scleroderma, obliterative bronchiolitis, Hermansky-Pudlak syndrome, asbestosis, silicosis, sarcoidosis, tumor stroma in lung disease, chronic obstructive pulmonary disease (COPD), emphysema, chronic bronchitis and asthma.

Also provided is an ADAMTS14 inhibitor for use in the treatment or prevention of a disease or condition characterised by fibrosis.

Also provided is the use of an ADAMTS14 inhibitor in the manufacture of a medicament for treating or preventing a disease or condition characterised by fibrosis.

Also provided is a method of treating or preventing a disease or condition characterised by fibrosis in a subject, comprising administering to a subject a therapeutically- or prophylactically-effective amount of an ADAMTS14 inhibitor.

In some embodiments, the disease or condition characterised by fibrosis comprises fibrosis of: an organ or tissue of the respiratory system, lung, bronchioles, alveoli, airways, nasal cavity, oral cavity, pharynx, larynx, trachea, bronchus, an organ or tissue of the cardiovascular system, heart, blood vessels, an organ or tissue of the gastrointestinal system, liver, bowel, small intestine, large intestine, colon, pancreas, skin, eye, an organ or tissue of the nervous system, brain, an organ or tissue of the urogenital system, ovaries, fallopian tubes, kidney, an organ or tissue of the musculoskeletal system, muscle tissue, or bone marrow.

In some embodiments, the disease or condition characterised by fibrosis is selected from: pulmonary fibrosis, interstitial lung disease (ILD), idiopathic interstitial pneumonia (IIP), idiopathic pulmonary fibrosis (IPF), cystic fibrosis, progressive massive fibrosis, scleroderma, obliterative bronchiolitis, Hermansky-Pudlak syndrome, asbestosis, silicosis, sarcoidosis, tumor stroma in lung disease, chronic obstructive pulmonary disease (COPD), emphysema, chronic bronchitis, asthma, chronic liver disease, liver fibrosis, cirrhosis, non-alcoholic fatty liver disease (NAFLD), steatohepatitis, non-alcoholic steatohepatitis (NASH), alcoholic liver disease (ALD), alcoholic fatty liver (AFL), alcoholic hepatitis, alcoholic steatohepatitis (ASH), primary biliary cirrhosis (PBC), schistosomal liver disease, hepatocellular carcinoma (HCC), hypertrophic cardiomyopathy (HCM), dilated cardiomyopathy (DCM), fibrosis of the atrium, atrial fibrillation, fibrosis of the ventricle, ventricular fibrillation, myocardial fibrosis, Brugada syndrome, myocarditis, endomyocardial fibrosis, myocardial infarction, fibrotic vascular disease, hypertension, hypertensive heart disease, arrhythmogenic right ventricular cardiomyopathy (ARVC), atherosclerosis, chronic pulmonary hypertension, AIDS-associated pulmonary hypertension, varicose veins, cerebral infarcts, tubulointerstitial fibrosis, glomerular fibrosis, renal fibrosis, nephritic syndrome, Alport's syndrome, HIV-associated nephropathy, polycystic kidney disease, Fabry's disease, diabetic nephropathy, chronic glomerulonephritis, nephritis associated with systemic lupus, pancreatic fibrosis, chronic pancreatitis, endometriosis, gliosis, Alzheimer's disease, multiple sclerosis, muscular dystrophy, Duchenne muscular dystrophy (DMD), Becker's muscular dystrophy (BMD), fibrotic myopathy, inflammatory bowel disease (IBD), Crohn's disease, microscopic colitis, primary sclerosing cholangitis (PSC), scleroderma, nephrogenic systemic fibrosis, Dupuytren's contracture, cutis keloid, Grave's opthalmopathy, epiretinal fibrosis, retinal fibrosis, subretinal fibrosis, subretinal fibrosis associated with macular degeneration, wet age-related macular degeneration (AMD), diabetic retinopathy, glaucoma, corneal fibrosis, post-surgical fibrosis, post-surgical fibrosis of the posterior capsule following cataract surgery, post-surgical fibrosis of the bleb following trabeculectomy for glaucoma, conjunctival fibrosis, subconjunctival fibrosis, arthrofibrosis, arthritis, adhesive capsulitis, progressive systemic sclerosis (PSS), chronic graft versus host disease (GVHD), fibrotic pre-neoplastic disease, fibrotic neoplastic disease, fibrosis induced by chemical insult, or fibrosis induced by environmental insult, fibrosis induced by cancer chemotherapy, fibrosis induced by pesticides, fibrosis induced by radiation, fibrosis induced by cancer radiotherapy, cancer, hepatocellular carcinoma, gastric cancer, esophageal cancer, head and neck cancer, colorectal cancer, pancreatic cancer, cervical cancer, vulvar cancer, mediastinal fibrosis, retroperitoneal fibrosis, myelofibrosis and Peyronie's disease.

In some embodiments, the disease or condition characterised by fibrosis comprises fibrosis of: an organ or tissue of the respiratory system, lung, bronchioles, alveoli, airways, nasal cavity, oral cavity, pharynx, larynx, trachea or bronchus.

In some embodiments, the disease or condition characterised by fibrosis is selected from: pulmonary fibrosis, interstitial lung disease (ILD), idiopathic interstitial pneumonia (IIP), idiopathic pulmonary fibrosis (IPF), cystic fibrosis, progressive massive fibrosis, scleroderma, obliterative bronchiolitis, Hermansky-Pudlak syndrome, asbestosis, silicosis, sarcoidosis, tumor stroma in lung disease, chronic obstructive pulmonary disease (COPD), emphysema, chronic bronchitis and asthma.

Also provided is a method of inhibiting TGFβ1-mediated signalling in a cell, comprising contacting a cell with an ADAMTS14 inhibitor.

Also provided is a method of inhibiting YAP-mediated signalling in a cell, comprising contacting a cell with an ADAMTS14 inhibitor.

Also provided is a method of increasing YAP degradation in a cell, comprising contacting a cell with an ADAMTS14 inhibitor.

Also provided is a method of inhibiting the generation of a pro-fibrotic fibroblast, comprising contacting a pro-fibrotic fibroblast precursor cell with an ADAMTS14 inhibitor.

Also provided is a method of inhibiting a process mediated by pro-fibrotic fibroblasts, comprising contacting a pro-fibrotic fibroblast or a pro-fibrotic fibroblast precursor cell with an ADAMTS14 inhibitor.

In some embodiments, the pro-fibrotic fibroblast is a myofibroblast.

Also provided is a method of inhibiting the generation of a myofibroblast, comprising contacting a myofibroblast precursor cell with an ADAMTS14 inhibitor.

Also provided is a method of inhibiting a process mediated by myofibroblasts, comprising contacting a myofibroblast or a myofibroblast precursor cell with an ADAMTS14 inhibitor.

In some embodiments, in accordance with the various aspects of the present disclosure, the ADAMTS14 inhibitor reduces gene and/or protein expression of ADAMTS14.

In some embodiments, in accordance with the various aspects of the present disclosure, the ADAMTS14 inhibitor is an inhibitory nucleic acid selected from: an siRNA, dsiRNA, miRNA, shRNA, pri-miRNA, pre-miRNA, saRNA, snoRNA, and an antisense oligonucleotide.

### DESCRIPTION

The present disclosure is based on the inventors' unexpected finding that ADAMTS14 is a key mediator of the fibrotic response. Antagonism of ADAMTS14 is shown to inhibit the generation of pro-fibrotic fibroblasts and their activity, resulting in a reduction in correlates of fibrosis.

The identification of ADAMTS14 is described in WO 03/042379 A2. This document includes speculative statements that nucleic acid encoding ADAMTS14, or alternatively its complementary strand, may be useful for the treatment of a wide range of diseases (see e.g. claim 23 of WO 03/042379 A2, as dependent from claim 16 and claim 1). However, WO 03/042379 A2 does not provide any data supporting the use of an inhibitor of ADAMTS14 expression/activity for the treatment of any of the specified diseases. WO 03/042379 A2 moreover discloses that treatment of fibroblasts with various profibroinflammatory cytokines (including IL-1β, TNFα and TGFβ) did not modify ADAMTS14 expression (see para [0076]). By contrast, ADAMTS1 is disclosed to be an inflammation-associated gene whose expression can be induced by IL-1, and expression of ADAMTS12, ADAMTS4 and ADAMTS5 is similarly disclosed to be upregulated by proinflammatory factors (see para [0079]). Thus, WO 03/042379 A2 suggests that ADAMTS14 is not an effector of fibroinflammatory processes, and implies that other, profibroinflammatory cytokine-responsive ADAMTS genes (i.e. ADAMTS1, ADAMTS12, ADAMTS4 and ADAMTS5) would be more promising targets for intervention for the treatment/prevention of fibrosis.

### ADAMTS14

The present disclosure relates particularly to the inhibition of gene and/or protein expression of ADAM Metallopeptidase with Thrombospondin Type 1 Motif 14 (ADAMTS14).

The structure and function of ADAMTS14 is described in e.g. in Colige et al., J Biol Chem. (2002) 277(8):5756-5766 and Bekhouche and Coliege, Matrix Biol. (2015) 44-46:46-53, both of which are hereby incorporated by reference in their entirety.

Alterative splicing of the mRNA transcribed from the human ADAMTS14 gene yields four isoforms: isoform A (UniProtKB: Q8WXS8-1, v2; SEQ ID NO:1); isoform B (UniProtKB: Q8WXS8-2; SEQ ID NO:2) in which the amino acid sequence corresponding to positions 1 to 67 of SEQ ID NO:1 are missing; isoform C (UniProtKB: Q8WXS8-3; SEQ ID NO:3) in which the amino acid sequence corresponding to positions 1 to 67 of SEQ ID NO:1 are missing, and wherein ‘MQG’ is inserted after ‘G’ at the position corresponding to position 368 of SEQ ID NO:1; and isoform D (UniProtKB: Q8WXS8-4; SEQ ID NO:4) in which ‘MQG’ is inserted after ‘G’ at the position corresponding to position 368 of SEQ ID NO:1.

The 1223 amino acid sequence of human ADAMTS14 isoform A comprises: an N-terminal signal peptide (positions 1 to 22 of SEQ ID NO:1, shown in SEQ ID NO:5), followed by a 230 amino acid propeptide (positions 23 to 252 of SEQ ID NO:1, shown in SEQ ID NO:6), and the mature protein region (positions 253 to 1223 of SEQ ID NO:1, shown in SEQ ID NO:7).

The mature protein region comprises an N-terminal peptidase M12B domain (positions 259 to 460 of SEQ ID NO:1, shown in SEQ ID NO:10), a disintegrin domain (positions 461 to 551 of SEQ ID NO:1, shown in SEQ ID NO: 11), four TSP type 1 repeats (positions 552 to 607, 847 to 907, 908 to 967 and 968 to 1022 of SEQ ID NO:1, shown in SEQ ID NOs:13, 14, 15 and 16 respectively) with a spacer domain provided between TSP type 1 repeats 1 and 2 (positions 730 to 846 of SEQ ID NO:1, shown in SEQ ID NO:17), a PLAC domain (positions 1059 to 1097 of SEQ ID NO:1, shown in SEQ ID NO:18), and a C-terminal proline-rich region (positions 1100-1223 of SEQ ID NO:1, shown in SEQ ID NO:19).

ADAMTS14 has been reported to display aminoprocollagen type I collagenase activity (Colige et al., supra).

In the experimental examples of the present disclosure, the inventors demonstrate that ADAMTS14 is implicated in nuclear localisation of YAP, and the regulation of the level and phosphorylation of YAP and TAZ in fibroblasts, as well as the YAP target genes and pro-fibrotic mediators CTGF and CYR61. ADAMTS14 is implicated in the TGFβ1-induced upregulation of expression of pro-fibrotic and metaplastic differentiation genes. ADAMTS14 is also shown to be involved in the TGFβ1-induced phosphorylation of SMAD2, and TGFβ1-induced localisation of SMAD2 to the nucleus. ADAMTS14 is also shown to be important for formation of α-SMA fibers in, and collagen deposition by, fibroblasts in response to TGFβ1.

In this specification, reference to ‘ADAMTS14’ encompasses: human ADAMTS14 isoforms A, B C and D, homologues of human ADAMTS14 isoforms A, B, C and D (i.e. encoded by the genome of a non-human animal), and variants thereof.

A homologue of human ADAMTS14 isoform A, B C or D may be from any animal. In some embodiments, a homologue of human ADAMTS14 isoform A, B C or D may be from a mammal. In some embodiments, the mammal may be a non-human mammal, e.g. a primate (e.g. a non-human primate, e.g. an animal of the genus Macaca (e.g. Macaca fascicularis, Macaca mulatta), e.g. a non-human hominid (e.g. Pan troglodytes)). In some embodiments, the mammal may be a rabbit, guinea pig, rat, mouse or animal of the order Rodentia, cat, dog, pig, sheep, goat, an animal of the order Bos (e.g. cattle), an animal of the family Equidae (e.g. horse) or donkey.

Homologues of a human ADAMTS14 isoform (e.g. isoform A, B, C or D) may optionally be characterised as having 70% or greater amino acid sequence identity, preferably one of 75%, 80%, 85%, 90%, 91%, 92%, 93%, 94%, 95%, 96%, 97%, 98%, 99% or 100% amino acid sequence identity to the amino acid sequence of the relevant isoform. Variants of a human ADAMTS14 isoform (e.g. isoform A, B, C or D) may optionally be characterised as having 70% or greater amino acid sequence identity, preferably one of 75%, 80%, 85%, 90%, 91%, 92%, 93%, 94%, 95%, 96%, 97%, 98%, 99% or greater amino acid sequence identity to the relevant isoform.

In some embodiments, ADAMTS14 according to the present disclosure comprises or consists of an amino acid sequence having at 70% or greater amino acid sequence identity, preferably one of 75%, 80%, 85%, 90%, 91%, 92%, 93%, 94%, 95%, 96%, 97%, 98%, 99% or 100% amino acid sequence identity to the amino acid sequence of SEQ ID NO:1, 2, 3 or 4.

In some embodiments, ADAMTS14 according to the present disclosure comprises an amino acid sequence having at 70% or greater amino acid sequence identity, preferably one of 75%, 80%, 85%, 90%, 91%, 92%, 93%, 94%, 95%, 96%, 97%, 98%, 99% or 100% amino acid sequence identity to the amino acid sequence of SEQ ID NO:8 or 9.

### ADAMTS14 Inhibitors

Aspects of the present disclosure are concerned with inhibitors of ADAMTS14.

An ‘ADAMTS14 inhibitor’ refers to any agent capable of inhibiting ADAMTS14 expression and/or function. That is, inhibitors of ADAMTS14 encompass agents that reduce the level of gene and/or protein expression of ADAMTS14, and agents which reduce an activity of ADAMTS14. It will be appreciated that the reduction in the preceding sentence refers to the level of expression/activity observed in the absence of such inhibition. In the present disclosure, inhibitors of ADAMTS14 may also be referred to as ‘antagonists’ of ADAMTS14, and similarly inhibition of ADAMTS14 may be referred to as ‘antagonism’ of ADAMTS14.

As used herein, an ‘activity’ or ‘function’ of or mediated by ADAMTS14 may refer to: cleavage of aminoprocollagen type I, nuclear localisation of YAP, nuclear localisation of TAZ, nuclear localisation of SMAD2, phosphorylation of SMAD2, upregulation of expression of one or more genes involved in fibrosis and/or metaplastic differentiation, formation of αSMA fibers and/or collagen I deposition (e.g. in/by fibroblasts).

In some embodiments, an ADAMTS14 inhibitor according to the present disclosure displays one or more of the following properties:
Reduces expression (e.g. gene and/or protein expression) of ADAMTS14;
Reduces the level of RNA encoding ADAMTS14;
Reduces transcription of nucleic acid encoding ADAMTS14;
Increases degradation of RNA encoding ADAMTS14;
Reduces the level of ADAMTS14 protein;
Reduces post-transcriptional processing (e.g. splicing, translation, post-translational processing) of RNA encoding ADAMTS14;
Increases degradation of ADAMTS14 protein;
Reduces the level of a correlate of ADAMTS14 activity;
Reduces cleavage of type I aminoprocollagen;
Reduces TGFβ1-mediated upregulation of the expression of one or more genes involved in fibrosis and/or metaplastic differentiation (e.g. one or more genes selected from ADAMTS14, ACTA2, COL1A1, FN1, FOXJ1, KRT5, MUC5AC, MUC5B, SCGB1A1, CTGF, CYR61, MMP7 and SOX2; e.g. in fibroblasts or epithelial cells);
Reduces the level of YAP (e.g. in fibroblasts);
Reduces nuclear localisation of YAP (e.g. in fibroblasts);
Increases degradation of YAP (e.g. in fibroblasts);
Increases the level of phosphorylated YAP (e.g. YAP phosphorylated at S397; e.g. in fibroblasts);
Reduces the level of a protein encoded by a gene whose expression is upregulated by YAP (e.g. in fibroblasts);
Reduces the level of TAZ (e.g. in fibroblasts);
Reduces nuclear localisation of TAZ (e.g. in fibroblasts);
Increases the level of phosphorylated TAZ (e.g. TAZ phosphorylated at S89; e.g. in fibroblasts);
Reduces the level of a protein encoded by a gene whose expression is upregulated by TAZ (e.g. in fibroblasts);
Reduces the level of CTGF (e.g. in fibroblasts);
Reduces the level of CYR61 (e.g. in fibroblasts);
Reduces TGFβ1-mediated signalling (e.g. in fibroblasts);
Reduces TGFβ1-mediated upregulation of the level of phosphorylated SMAD2 (e.g. in fibroblasts);
Reduces TGFβ1-mediated upregulation localisation of SMAD2 to the nucleus (e.g. in fibroblasts);
Reduces TGFβ1-mediated upregulation of the level of αSMA (e.g. in fibroblasts); and/or
Reduces TGFβ1-mediated production of a component of extracellular matrix (e.g. type 1 collagen I; e.g. in/by fibroblasts);
Reduces extracellular matrix stiffness (e.g. of extracellular matrix produced by fibroblasts or epithelial cells in culture in vitro); and
Reduces the stiffness of a tissue/organ (e.g. lung, liver, skin, kidney), e.g. a tissue/organ comprising fibrosis.

It will be appreciated that a given ADAMTS14 inhibitor may display more than one of the properties recited in the preceding paragraph. A given ADAMTS14 inhibitor may be evaluated for the properties recited in the preceding paragraph using suitable assays. For example, the assays may be e.g. in vitro assays, optionally cell-based assays or cell-free assays. In some embodiments, the assays may be e.g. in vivo assays, i.e. performed in non-human animals. In some embodiments, the assays may be e.g. ex vivo assays, i.e. performed using cells/tissue/an organ obtains from a subject.

Where assays are cell-based assays, they may comprise treating cells with a given agent in order to determine whether the agent displays one or more of the recited properties. Assays may employ species labelled with detectable entities in order to facilitate their detection. Assays may comprise evaluating the recited properties following treatment of cells separately with a range of quantities/concentrations of a given agent (e.g. a dilution series). It will be appreciated that the cells employed in such are preferably cells that express ADAMTS14, e.g. fibroblasts (e.g. lung fibroblasts).

In accordance with the various aspects and embodiments of the present disclosure, a fibroblast/fibroblasts may be resident in or derived from a tissue or organ of interest. In some embodiments, a fibroblast/fibroblasts may be resident in or derived from the lung, bronchioles, alveoli, airways, nasal cavity, oral cavity, pharynx, larynx, trachea, bronchi, heart, kidney, liver, skeletal muscle, blood vessels, eye, skin, pancreas, bowel, small intestine, large intestine, colon, joints, brain, or bone marrow. In some embodiments, a fibroblast/fibroblasts may be resident in or derived from the lung.

In some embodiments, the assays may comprise treating cells to upregulate ADAMTS14 expression and/or activity. For example, the assays may comprise treating the cells with TGFβ1 (e.g. at a final concentration of 5 ng/ml), or with IPF-relevant cytokine cocktail (IPF-RC, described e.g. in Schruf et al. FASEB J. (2020) 34(6):7825-7846; e.g. at a 1:100 dilution).

Analysis of the results of such assays may comprise determining the concentration at which 50% of the maximal level of the relevant activity is attained. The concentration of a given agent at which 50% of the maximal level of the relevant activity is attained may be referred to as the ‘half-maximal effective concentration’ of the agent in relation to the relevant activity, which may also be referred to as the ‘EC₅₀’. By way of illustration, the EC₅₀ of a given agent for increasing degradation of RNA encoding ADAMTS14 may be the concentration of the agent at which 50% of the maximal degradation of RNA encoding ADAMTS14 is achieved.

Depending on the property, the EC₅₀ may also be referred to as the ‘half-maximal inhibitory concentration’ or ‘IC₅₀’, this being the concentration of the agent at which 50% of the maximal level of inhibition of a given property is observed. By way of illustration, the IC₅₀ of a given agent for reducing gene expression of ADAMTS14 may be the concentration of the agent at which 50% of the maximal level of inhibition of expression of ADAMTS14 is achieved.

ADAMTS14 inhibitors capable of reducing gene expression of ADAMTS14 and/or reducing the level of RNA encoding ADAMTS14 and/or reducing transcription of nucleic acid encoding ADAMTS14 and/or increasing degradation of RNA encoding ADAMTS14 may be identified using assays comprising detecting and/or quantifying the level of RNA encoding ADAMTS14. Such assays may comprise quantifying RNA encoding ADAMTS14 by RT-qPCR, northern blot, etc., which are techniques well known to the skilled person. The methods may employ primers and/or probes for the detection and/or quantification of RNA encoding ADAMTS14. Such assays may comprise contacting cells that express ADAMTS14 (e.g. fibroblasts) in in vitro culture with a putative ADAMTS14 inhibitor, and subsequently (e.g. after an appropriate period of time, i.e. a period of time sufficient for a change in the level of RNA encoding ADAMTS14 to be observed) measuring the level of RNA encoding ADAMTS14. Such assays may further comprise comparing the level of RNA encoding ADAMTS14 in cells treated with the putative ADAMTS14 inhibitor to the level of RNA encoding ADAMTS14 detected in a control condition in which cells of the same type are subjected to the same conditions, except that instead of being treated with the putative ADAMTS14 inhibitor they are untreated, or otherwise treated with a negative control agent known not to affect the level of RNA encoding ADAMTS14.

Reduced transcription of nucleic acid encoding ADAMTS14 may be a consequence of inhibition of assembly and/or activity of factors required for transcription of the DNA encoding ADAMTS14. Increased degradation of RNA encoding ADAMTS14 may be a consequence of increased enzymatic degradation of RNA encoding ADAMTS14, e.g. as a consequence of RNA interference (RNAi), and/or reduced stability of RNA encoding ADAMTS14.

Herein, ‘contacting’ cells with a given agent (e.g. a putative ADAMTS14 inhibitor) may comprise applying the agent to, and/or mixing the agent with, the cells. In some embodiments, the putative ADAMTS14 inhibitor is provided to the cells in combination with one or more further agents for facilitating introduction of the putative ADAMTS14 inhibitor into the cells, and/or for facilitating uptake of the putative ADAMTS14 inhibitor by the cells. For example, in embodiments wherein a putative ADAMTS14 inhibitor is, or is encoded by, one or more nucleic acids, the cells may be contacted with the nucleic acid(s) and an agent for facilitating introduction of the nucleic acid(s) into the cells, e.g. by transfection or transduction.

In some embodiments, a putative ADAMTS14 inhibitor may be evaluated for its ability to reduce gene expression of ADAMTS14 (e.g. in fibroblasts) as described in Example 1 herein.

ADAMTS14 inhibitors capable of reducing post-transcriptional processing of RNA encoding ADAMTS14 (e.g. reducing normal splicing of pre-mRNA encoding ADAMTS14) may be identified using assays comprising detecting and/or quantifying the level of RNA (e.g. mature mRNA) encoding one or more isoforms of ADAMTS14. Such assays may comprise quantifying RNA (e.g. mature mRNA) encoding one or more isoforms of ADAMTS14 by RT-qPCR. The methods may employ primers and/or probes for the detection and/or quantification of mature mRNA produced by canonical splicing of pre-mRNA transcribed from a gene encoding ADAMTS14, and/or primers and/or probes for the detection and/or quantification of mature mRNA produced by alternative splicing of pre-mRNA transcribed from a gene encoding ADAMTS14. Mature mRNA produced by canonical splicing of pre-mRNA transcribed from a gene encoding ADAMTS14 may be mature mRNA encoding the major isoform produced by expression of the gene encoding ADAMTS14. The major isoform may be the most commonly produced/detected isoform. For example, mature mRNA produced by canonical splicing of pre-mRNA transcribed from human ADAMTS14 may be mature mRNA encoding human ADAMTS14 isoform A (i.e. having the amino acid sequence shown in SEQ ID NO:1). Mature mRNA produced by alternative splicing of pre-mRNA transcribed from a gene encoding ADAMTS14 may be mature mRNA encoding an isoform other than the major isoform produced by expression of the gene encoding ADAMTS14. For example, mature mRNA produced by alternative splicing of pre-mRNA transcribed from human ADAMTS14 may be mature mRNA encoding an isoform of human ADAMTS14 other than isoform A (i.e. having an amino acid sequence non-identical to SEQ ID NO:1); e.g. mature mRNA encoding human ADAMTS14 isoform B, C or D. Such assays may comprise contacting cells that express ADAMTS14 (e.g. fibroblasts) in in vitro culture with a putative ADAMTS14 inhibitor, and subsequently (e.g. after an appropriate period of time, i.e. a period of time sufficient for an effect on post-transcriptional processing of RNA encoding ADAMTS14 (e.g. splicing of pre-mRNA encoding ADAMTS14) to be observed) measuring the level of mature mRNA encoding one or more isoforms of ADAMTS14. Such assays may further comprise comparing the level of mature mRNA encoding one or more isoforms of ADAMTS14 in cells treated with the putative ADAMTS14 inhibitor to the level of mature mRNA encoding one or more isoforms of ADAMTS14 detected in a control condition in which cells of the same type are subjected to the same conditions, except that instead of being treated with the putative ADAMTS14 inhibitor they are untreated, or otherwise treated with a negative control agent known not to affect splicing of pre-mRNA encoding ADAMTS14.

A reduction in the level of canonical splicing of pre-mRNA encoding ADAMTS14 may be a consequence of inhibition of assembly and/or activity of factors required for canonical splicing. Reduced translation of mRNA encoding ADAMTS14 may be a consequence of inhibition of assembly and/or activity of factors required for translation. Reduced post-translational processing (e.g. enzymatic processing, folding) of ADAMTS14 may be a consequence of inhibition of assembly and/or activity of factors required for post-translational processing of ADAMTS14. Increased degradation of ADAMTS14 protein may be a consequence of increased enzymatic (e.g. protease-mediated) degradation of ADAMTS14 protein.

ADAMTS14 inhibitors capable of reducing the level of ADAMTS14 protein and/or increasing degradation of ADAMTS14 protein and/or reducing translation of mRNA encoding ADAMTS14 may be identified using assays comprising detecting the level of ADAMTS14 protein, e.g. using techniques well known to the skilled person, such as antibody/reporter-based methods (western blot, ELISA, immunohisto/cytochemistry, etc.). The methods may employ antibodies specific for ADAMTS14. Such assays may comprise contacting cells that express ADAMTS14 (e.g. fibroblasts) in in vitro culture with a putative ADAMTS14 inhibitor, and subsequently (e.g. after an appropriate period of time, i.e. a period of time sufficient for a change in the level of ADAMTS14 protein to be observed) measuring the level of ADAMTS14 protein. Such assays may further comprise comparing the level of ADAMTS14 protein in cells treated with the putative ADAMTS14 inhibitor to the level of ADAMTS14 protein detected in a control condition in which cells of the same type are subjected to the same conditions, except that instead of being treated with the putative ADAMTS14 inhibitor they are untreated, or otherwise treated with a negative control agent known not to affect the level of ADAMTS14 protein.

A reduction in the level of ADAMTS14 protein may e.g. be the result of a reduction in the level of RNA encoding ADAMTS14, reduced post-transcriptional processing of RNA encoding ADAMTS14, or increased degradation of ADAMTS14 protein.

In some embodiments, a putative ADAMTS14 inhibitor may be evaluated for its ability to reduce the level of ADAMTS14 protein (e.g. in fibroblasts) as described in Example 1 herein.

ADAMTS14 inhibitors capable of reducing the level of a function of ADAMTS14 (e.g. a function of ADAMTS14 as described hereinabove) may be identified using assays comprising detecting the level of the relevant function. Detecting the level of a given function may comprise detecting and/or quantifying a correlate of the function. By way of illustration, detecting the level of cleavage of type I aminoprocollagen may comprise detecting and/or quantifying the level of cleaved and/or uncleaved type I aminoprocollagen. Such assays may comprise contacting cells that express ADAMTS14 (e.g. fibroblasts) in in vitro culture with a putative ADAMTS14 inhibitor, and subsequently (e.g. after an appropriate period of time, i.e. a period of time sufficient for a reduction in the level of the relevant function and/or a correlate thereof to be observed) measuring the level of the relevant function and/or a correlate thereof. Such assays may further comprise comparing the level of a function of ADAMTS14 in cells treated with the putative ADAMTS14 inhibitor to the level of the function of ADAMTS14 detected in a control condition in which cells of the same type are subjected to the same conditions, except that instead of being treated with the putative ADAMTS14 inhibitor they are untreated, or otherwise treated with a negative control agent known not to affect the level of the relevant function of ADAMTS14.

ADAMTS14 inhibitors capable of reducing cleavage of type I aminoprocollagen may be identified using assays comprising detecting and/or quantifying the level of cleaved and/or uncleaved type I aminoprocollagen. Such assays may comprise contacting cells that express ADAMTS14 (e.g. fibroblasts) in in vitro culture with a putative ADAMTS14 inhibitor, and subsequently (e.g. after an appropriate period of time, i.e. a period of time sufficient for a reduction in the cleavage of type I aminoprocollagen and/or a correlate thereof to be observed) measuring the level of cleaved and/or uncleaved type I aminoprocollagen. Such assays may further comprise comparing the level of cleavage of type I aminoprocollagen in cells treated with the putative ADAMTS14 inhibitor to the level detected in a control condition in which cells of the same type are subjected to the same conditions, except that instead of being treated with the putative ADAMTS14 inhibitor they are untreated, or otherwise treated with a negative control agent known not to affect the level of cleavage of type I aminoprocollagen.

ADAMTS14 inhibitors capable of reducing upregulation of the expression of one or more genes involved in fibrosis and/or metaplastic differentiation (e.g. one or more genes selected from ADAMTS14, ACTA2, COL1A1, FN1, FOXJ1, KRT5, MUC5AC, MUC5B, SCGB1A1, CTGF, CYR61, MMP7 and SOX2; e.g. in fibroblasts or epithelial cells) in response to stimulation with IPF-RC may be identified using assays comprising detecting and/or quantifying the level of expression of the relevant gene(s). Such assays may comprise contacting cells that express ADAMTS14 (e.g. fibroblasts) in in vitro culture with a putative ADAMTS14 inhibitor and TGFβ1, and subsequently (e.g. after an appropriate period of time, i.e. a period of time sufficient for a reduction in the level of expression of the relevant gene(s) to be observed) measuring the level of expression of the relevant gene(s), e.g. by detecting and/or quantifying the level of RNA encoding a product of the relevant gene(s). Such assays may further comprise comparing the level of expression of the relevant gene(s) in cells treated with the putative ADAMTS14 inhibitor to the level detected in a control condition in which cells of the same type are subjected to the same conditions, except that instead of being treated with the putative ADAMTS14 inhibitor they are untreated, or otherwise treated with a negative control agent known not to affect the level of expression of the relevant gene(s).

Genes whose expression is upregulated by YAP/TAZ are described e.g. in Moya and Halder, Nat. Rev. Mol. Cell Biol. (2019) 20:211-226 (which is hereby incorporated by reference in its entirety), and include e.g. CTGF, CYR61, BIRC5, AREG, AMOTL1 and ANKRD1.

In some embodiments, a putative ADAMTS14 inhibitor may be evaluated for its ability to reduce upregulation of the expression of one or more genes involved in fibrosis and/or metaplastic differentiation (e.g. one or more genes selected from ADAMTS14, ACTA2, COL1A1, FN1, FOXJ1, KRT4, MUC5AC, MC5B, SCGB1A1, CTGF, CYR61, MMP7 and SOX2; e.g. in fibroblasts or epithelial cells) in response to stimulation with IPF-RC as described in Example 1 herein.

ADAMTS14 inhibitors capable of reducing the level of a given protein (e.g. YAP, TAZ, SMAD2, collagen I, αSMA, proteins encoded by genes whose expression is upregulated by YAP/TAZ) or a specific post-translationally modified version thereof (e.g. phosphorylated SMAD2) may be identified using assays comprising detecting and/or quantifying the level of the relevant protein/post-translationally modified version thereof, e.g. using techniques well known to the skilled person, such as antibody/reporter-based methods (western blot, ELISA, immunohisto/cytochemistry, etc.). The methods may employ antibodies specific for the relevant protein or post-translationally modified version thereof. Such assays may comprise contacting cells that express ADAMTS14 (e.g. fibroblasts) in in vitro culture with a putative ADAMTS14 inhibitor and optionally TGFβ1, and subsequently (e.g. after an appropriate period of time, i.e. a period of time sufficient for a change in the level of the relevant protein/post-translationally modified version thereof to be observed) measuring the level of the relevant protein/post-translationally modified version thereof. Such assays may further comprise comparing the level of the relevant protein/post-translationally modified version thereof in cells treated with the putative ADAMTS14 inhibitor to the level detected in a control condition in which cells of the same type are subjected to the same conditions, except that instead of being treated with the putative ADAMTS14 inhibitor they are untreated, or otherwise treated with a negative control agent known not to affect the level of the relevant protein/post-translationally modified version thereof.

In some embodiments, a putative ADAMTS14 inhibitor may be evaluated for its ability to reduce the level of YAP, TAZ, SMAD2, collagen I, αSMA, phosphorylated SMAD2, a protein encoded by a gene whose expression is upregulated by YAP or a protein encoded by a gene whose expression is upregulated by TAZ (e.g. in fibroblasts, e.g. following stimulation with TGFβ1) as described in Example 1 herein.

ADAMTS14 inhibitors capable of reducing TGFβ1-mediated signalling may be identified using assays comprising detecting and/or quantifying the level of a correlate of TGFβ1-mediated signalling, e.g. using techniques well known to the skilled person, such as antibody/reporter-based methods (western blot, ELISA, immunohisto/cytochemistry, etc.). The methods may employ antibodies specific for a correlate of TGFβ1-mediated signalling, e.g. phosphorylated SMAD2. Such assays may comprise contacting cells that express ADAMTS14 (e.g. fibroblasts) in in vitro culture with a putative ADAMTS14 inhibitor and TGFβ1, and subsequently (e.g. after an appropriate period of time, i.e. a period of time sufficient for inhibition of TGFβ1-mediated signalling to be observed) measuring the level of the correlate of TGFβ1-mediated signalling. Such assays may further comprise comparing the level of the correlate of TGFβ1-mediated signalling in cells treated with the putative ADAMTS14 inhibitor to the level detected in a control condition in which cells of the same type are subjected to the same conditions, except that instead of being treated with the putative ADAMTS14 inhibitor they are untreated, or otherwise treated with a negative control agent known not to affect TGFβ1-mediated signalling.

In some embodiments, a putative ADAMTS14 inhibitor may be evaluated for its ability to reduce TGFβ1-mediated signalling (e.g. in fibroblasts) as described in Example 1 herein.

ADAMTS14 inhibitors capable of increasing or reducing the proportion of a given protein which is a specific post-translationally modified version thereof may be identified using assays comprising detecting and/or quantifying the level of the relevant protein and/or the post-translationally modified version thereof, e.g. using techniques well known to the skilled person, such as antibody/reporter-based methods (western blot, ELISA, immunohisto/cytochemistry, etc.). The methods may employ antibodies specific for the relevant protein and antibodies specific for the post-translationally modified version thereof. Such assays may comprise contacting cells that express ADAMTS14 (e.g. fibroblasts) in in vitro culture with a putative ADAMTS14 inhibitor and optionally TGFβ1, and subsequently (e.g. after an appropriate period of time, i.e. a period of time sufficient for a change in the proportion of the given protein which is a specific post-translationally modified version thereof to be observed) measuring the level of the relevant protein and the level of the post-translationally modified version thereof. Such assays may further comprise determining the proportion of the relevant protein which is the post-translationally modified version thereof. Such assays may further comprise comparing the proportion of the relevant protein which is the post-translationally modified version thereof determined in cells treated with the putative ADAMTS14 inhibitor to the proportion determined in a control condition in which cells of the same type are subjected to the same conditions, except that instead of being treated with the putative ADAMTS14 inhibitor they are untreated, or otherwise treated with a negative control agent known not to affect the proportion of the relevant protein which is the post-translationally modified version thereof.

ADAMTS14 inhibitors capable of reducing nuclear localisation of a given protein or a specific post-translationally modified version thereof (e.g. YAP, TAZ, SMAD2, phosphorylated SMAD2) and/or reducing the proportion of a given protein or a specific post-translationally modified version thereof localised to the nucleus may be identified using assays comprising detecting, quantifying and/or determining the proportion of the relevant protein/post-translationally modified version thereof localised to the nucleus, e.g. using techniques well known to the skilled person, such as antibody/reporter-based methods (western blot, ELISA, immunohisto/cytochemistry, etc.). The subcellular localisation of a given protein/post-translationally modified version thereof may be analysed e.g. by immunocytochemistry, or western blot of extracts prepared from different cellular fractions, and may employ organelle (e.g. nucleus) markers and/or labelled proteins of known subcellular localisation. Such assays may comprise contacting cells that express ADAMTS14 (e.g. fibroblasts) in in vitro culture with a putative ADAMTS14 inhibitor and optionally TGFβ1, and subsequently (e.g. after an appropriate period of time, i.e. a period of time sufficient for a change in subcellular localisation of the given protein/post-translationally modified version thereof to be observed) detecting and/or quantifying the given protein/post-translationally modified version thereof localised to the nucleus and/or not localised to the nucleus (e.g. localised to the cytoplasm). Such assays may further comprise determining the proportion of the relevant protein/post-translationally modified version thereof localised to the nucleus. Such assays may further comprise comparing the proportion of the relevant protein/post-translationally modified version thereof localised to the nucleus in cells treated with the putative ADAMTS14 inhibitor to the proportion determined in a control condition in which cells of the same type are subjected to the same conditions, except that instead of being treated with the putative ADAMTS14 inhibitor they are untreated, or otherwise treated with a negative control agent known not to affect localisation of the relevant protein/post-translationally modified version thereof to the nucleus.

In some embodiments, an ADAMTS14 inhibitor according to the present disclosure is capable of reducing extracellular matrix stiffness. In some embodiments, the extracellular matrix may be extracellular matrix produced by fibroblasts or epithelial cells in culture in vitro. In some embodiments, an ADAMTS14 inhibitor is capable of reducing the stiffness of a tissue or organ. In some embodiments, the tissue/organ may be, or may be from, the lung, liver, skin or kidney. In some embodiments, the tissue/organ is a tissue/organ comprising fibrosis (i.e. in some embodiments the tissue/organ is fibrotic).

The stiffness of extracellular matrix, or the stiffness of a given tissue/organ, can be evaluated e.g. by determining Young's modulus, e.g. as described in Akhmanova et al., Stem Cells Int. (2015) 2015:167025, which is hereby incorporated by reference in its entirety. Methods for evaluating Young's modulus are summarised e.g. in Table 2 of Akhmanova et al., and included analysis by ultrasound elastography, atomic force microscopy or millimeter indentation.

In some embodiments, an ADAMTS14 inhibitor according to the present disclosure is capable of reducing expression of a gene encoding ADAMTS14/reducing the level of RNA encoding ADAMTS14/reducing transcription of nucleic acid encoding ADAMTS14/reducing the level of ADAMTS14 protein/reducing post-transcriptional processing of RNA encoding ADAMTS14/reducing normal splicing of pre-mRNA encoding ADAMTS14/reducing translation of mRNA encoding ADAMTS14/reducing the level of a correlate of ADAMTS14 activity/reducing cleavage of type I aminoprocollagen/reducing TGFβ1-mediated upregulation of the expression of one or more genes involved in fibrosis and/or metaplastic differentiation/reducing the level of YAP/reducing nuclear localisation of YAP/reducing the level of a protein encoded by a gene whose expression is upregulated by YAP/reducing the level of TAZ/reducing nuclear localisation of TAZ/reducing the level of a protein encoded by a gene whose expression is upregulated by TAZ/reducing the level of CTGF/reducing the level of CYR61/reducing TGFβ1-mediated signalling/reducing upregulation of the level of phosphorylated SMAD2 in response to stimulation with TGFβ1/reducing TGFβ1-mediated upregulation of localisation of SMAD2 to the nucleus/reducing TGFβ1-mediated upregulation of the level of αSMA/reducing TGFβ1-mediated production of a component of extracellular matrix (e.g. type 1 collagen)/reducing extracellular matrix stiffness/reducing the stiffness of a tissue or organ to less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the level observed in the absence of the ADAMTS14 inhibitor, or in the presence of the same quantity of a control agent known not to possess such inhibitory activity, in a given assay.

In some embodiments, an ADAMTS14 inhibitor according to the present disclosure is capable of reducing expression of a gene encoding ADAMTS14/reducing the level of RNA encoding ADAMTS14/reducing transcription of nucleic acid encoding ADAMTS14/reducing the level of ADAMTS14 protein/reducing post-transcriptional processing of RNA encoding ADAMTS14/reducing normal splicing of pre-mRNA encoding ADAMTS14/reducing translation of mRNA encoding ADAMTS14/reducing the level of a correlate of ADAMTS14 activity/reducing cleavage of type I aminoprocollagen/reducing TGFβ1-mediated upregulation of the expression of one or more genes involved in fibrosis and/or metaplastic differentiation/reducing the level of YAP/reducing nuclear localisation of YAP/reducing the level of a protein encoded by a gene whose expression is upregulated by YAP/reducing the level of TAZ/reducing nuclear localisation of TAZ/reducing the level of a protein encoded by a gene whose expression is upregulated by TAZ/reducing the level of CTGF/reducing the level of CYR61/reducing TGFβ1-mediated signalling/reducing upregulation of the level of phosphorylated SMAD2 in response to stimulation with TGFβ1/reducing TGFβ1-mediated upregulation of localisation of SMAD2 to the nucleus/reducing TGFβ1-mediated upregulation of the level of αSMA/reducing TGFβ1-mediated production of a component of extracellular matrix (e.g. type 1 collagen)/reducing extracellular matrix stiffness/reducing the stiffness of a tissue or organ to less than 100%, e.g. one of ≤99%, ≤95%, ≤90%, ≤85%, ≤80%, ≤75%, ≤70%, ≤65%, ≤60%, ≤55%, ≤50%, ≤45%, ≤40%, ≤35%, ≤30%, ≤25%, ≤20%, ≤15%, ≤10%, ≤5%, or ≤1% of the level observed in the absence of the ADAMTS14 inhibitor, or in the presence of the same quantity of a control agent known not to possess such inhibitory activity, in a given assay.

Preferred levels of reduction in accordance with the preceding two paragraphs are reduction to less than 0.5 times/≤50%, e.g. one of less than 0.4 times/≤40%, less than 0.3 times/≤30%, less than 0.2 times/≤20%, less than 0.15 times/≤15%, or less than 0.1 times/≤10%.

In some embodiments, an ADAMTS14 inhibitor according to the present disclosure is capable of increasing degradation of RNA encoding ADAMTS14/increasing degradation of ADAMTS14 protein/increasing degradation of YAP/increasing the level of phosphorylated YAP (e.g. YAP phosphorylated at S397)/increasing degradation of TAZ/increasing the level of phosphorylated TAZ (e.g. TAZ phosphorylated at S89) to more than 1 times, e.g. one of ≥1.01 times, ≥1.02 times, ≥1.03 times, 1.04 times, ≥1.05 times, ≥1.1 times, ≥1.2 times, ≥1.3 times, 1.4 times, ≥1.5 times, ≥1.6 times, ≥1.7 times, ≥1.8 times, ≥1.9 times, ≥2 times, ≥3 times, ≥4 times, ≥5 times, ≥6 times, ≥7 times, ≥8 times, ≥9 times or ≥10 times the level observed in the absence of the ADAMTS14 inhibitor, or in the presence of the same quantity of a control agent known not to possess such activity, in a given assay.

In some embodiments, an ADAMTS14 inhibitor according to the present disclosure prevents or silences expression of a gene encoding ADAMTS14. In some embodiments, an ADAMTS14 inhibitor according to the present disclosure prevents or silences expression of ADAMTS14 at the protein level. As used herein, expression of a given gene/protein may be considered to be ‘prevented’ or ‘silenced’ where the level of expression is reduced to less than 0.1 times/≤10% of the level observed in the absence of the putative ADAMTS14 inhibitor, or in the presence of the same quantity of a control agent known not to inhibit expression of the relevant gene(s)/protein(s).

In preferred embodiments, an ADAMTS14 inhibitor according to the present disclosure inhibits greater than 50%, e.g. one of ≥60%, ≥61%, ≥62%, ≥63%, ≥64%, ≥65%, ≥66%, ≥67%, ≥68%, ≥69%, ≥70%, ≥71%, ≥72%, ≥73%, ≥74%, ≥75%, ≥76%, ≥77%, ≥78%, ≥79%, ≥80%, ≥81%, ≥82%, ≥83%, ≥84%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100% of the gene and/or protein expression of ADAMTS14 observed in the absence of the ADAMTS14 inhibitor, or in the presence of the same quantity of a control agent known not to inhibit gene and/or protein expression of ADAMTS14, in a given assay.

In preferred embodiments, an ADAMTS14 inhibitor according to the present disclosure inhibits greater than 50%, e.g. one of ≥60%, ≥61%, ≥62%, ≥63%, ≥64%, ≥65%, ≥66%, ≥67%, ≥68%, ≥69%, ≥70%, ≥71%, ≥72%, ≥73%, ≥74%, ≥75%, ≥76%, ≥77%, ≥78%, ≥79%, ≥80%, ≥81%, ≥82%, ≥83%, ≥84%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100% of the gene expression of ADAMTS14 (e.g. as determined by qRT-PCR) observed in the absence of the ADAMTS14 inhibitor, or in the presence of the same quantity of a control agent known not to inhibit gene expression of ADAMTS14, in a given assay (e.g. the assay described in Example 1 herein).

In preferred embodiments, an ADAMTS14 inhibitor according to the present disclosure inhibits greater than 50%, e.g. one of ≥60%, ≥61%, ≥62%, ≥63%, ≥64%, ≥65%, ≥66%, ≥67%, ≥68%, ≥69%, ≥70%, ≥71%, ≥72%, ≥73%, ≥74%, ≥75%, ≥76%, ≥77%, ≥78%, ≥79%, ≥80%, ≥81%, ≥82%, ≥83%, ≥84%, ≥85%, 86%, ≥87%, ≥88%, ≥89%, ≥90%, ≥91%, ≥92%, ≥93%, ≥94%, ≥95%, ≥96%, ≥97%, ≥98%, ≥99% or 100% of the protein expression of ADAMTS14 (e.g. as determined by ELISA) observed in the absence of the ADAMTS14 inhibitor, or in the presence of the same quantity of a control agent known not to inhibit protein expression of ADAMTS14, in a given assay (e.g. the assay described in Example 1 herein).

In some embodiments, an ADAMTS14 inhibitor according to the present disclosure is selected from: an agent capable of reducing gene and/or protein expression of ADAMTS14, and an agent capable of binding to and inhibiting the activity of ADAMTS14. Agents capable of reducing gene and/or protein expression of ADAMTS14 and agents capable of inhibiting the activity of ADAMTS14 can be identified using the assays described hereinabove.

Agents capable of binding to and inhibiting the activity of ADAMTS14 may e.g. be peptides/polypeptides, nucleic acids or small molecules. Agents capable of binding to and inhibiting the activity of ADAMTS14 may be identified by screening libraries of such molecules for the ability to bind to ADAMTS14 and inhibit an activity of ADAMTS14.

Peptides/polypeptides capable of binding to and inhibiting the activity of ADAMTS14 may be e.g. antibodies (immunoglobulins, e.g. monoclonal antibodies, polyclonal antibodies, monospecific antibodies, multispecific (e.g. bispecific) antibodies) antibody-derived molecules (e.g. antigen-binding fragments of antibodies (e.g. Fv, scFv, Fab, scFab, F(ab′)2, Fab2, diabodies, triabodies, scFv-Fc, minibodies, single domain antibodies (e.g. VhH), etc.), peptide aptamers, thioredoxins, monobodies, anticalin, Kunitz domains, avimers, knottins, fynomers, atrimers, DARPins, affibodies, nanobodies (i.e. single-domain antibodies (sdAbs)) affilins, armadillo repeat proteins (ArmRPs) or OBodies (reviewed e.g. in Reverdatto et al., Curr Top Med Chem. 2015; 15(12): 1082-1101, which is hereby incorporated by reference in its entirety; see also e.g. Boersma et al., J Biol Chem (2011) 286:41273-85 and Emanuel et al., Mabs (2011) 3:38-48).

Nucleic acids capable of binding to and inhibiting the activity of ADAMTS14 may be e.g. nucleic acid aptamers. Nucleic acid aptamers are reviewed e.g. in Zhou and Rossi, Nat Rev Drug Discov. (2017) 16(3):181-202, and may be identified and/or produced by the method of Systematic Evolution of Ligands by EXponential enrichment (SELEX), or by developing SOMAmers (slow off-rate modified aptamers) (Gold L et al. (2010) PLoS ONE 5(12):e15004). Nucleic acid aptamers may comprise DNA and/or RNA, and may be single stranded or double stranded. They may comprise chemically-modified nucleic acids, for example in which the sugar and/or phosphate and/or base is chemically modified, e.g. to improve the stability of the aptamer and/or to increase resistance of the aptamer to degradation.

ADAMTS14-binding aptamers include Cat. No. CTApt-1291 from Creative Biolabs.

As used herein, a ‘small molecule’ refers to a low molecular weight (<1000 daltons, typically between ˜300-700 daltons) organic compound. Small molecule inhibitors of ADAMTS14 may be identified by screening libraries of such small molecules for the ability to bind to an inhibit an activity of ADAMTS14.

Agents capable of reducing gene and/or protein expression of ADAMTS14 include e.g. inhibitory nucleic acids and site-specific nuclease (SSN) systems.

Inhibitory nucleic acids according to the present disclosure may comprise or consist of DNA and/or RNA. Inhibitory nucleic acids may be single-stranded (e.g. in the case of antisense oligonucleotides (e.g. gapmers)). Inhibitory nucleic acids may be double-stranded or may comprise double-stranded region(s) (e.g. in the case of siRNA, shRNA, etc.). Inhibitory nucleic acids may comprise both double-stranded and single-stranded regions (e.g. in the case of shRNA and pre-miRNA molecules, which are double-stranded in the stem region of the hairpin structure, and single-stranded in the loop region of the hairpin structure).

In some embodiments, an inhibitory nucleic acid according to the present disclosure may be an antisense nucleic acid as described herein. In some embodiments, an inhibitory nucleic acid may comprise an antisense nucleic acid as described herein. In some embodiments, an inhibitory nucleic acid may encode an antisense nucleic acid as described herein.

As used herein, an ‘antisense nucleic acid’ refers to a nucleic acid (e.g. DNA or RNA) that is complementary to at least a portion of a target nucleotide sequence (e.g. of RNA encoding ADAMTS14). Antisense nucleic acids according to the present disclosure are preferably single-stranded nucleic acids, and bind via complementary Watson-Crick base-pairing to a target nucleotide sequence. Complementary base-pairing may involve hydrogen bonding between complementary base pairs. Antisense nucleic acids may be provided as single-stranded molecules, as for example in the case of antisense oligonucleotides, or may be comprised in double-stranded molecular species, as for example in the case of siRNA, shRNA and pre-miRNA molecules.

Complementary base-pairing between the antisense nucleic acid and its target nucleotide sequence may be complete. In such embodiments the antisense nucleic acid comprises, or consists of, the reverse complement of its target nucleotide sequence, and complementary base-pairing occurs between each nucleotide of the target nucleotide sequence and complementary nucleotides in the antisense nucleic acid. Alternatively, complementary base-pairing between the antisense nucleic acid and its target nucleotide sequence may be incomplete/partial. In such embodiments complementary base-pairing occurs between some, but not all, nucleotides of the target nucleotide sequence and complementary nucleotides in the antisense nucleic acid.

Such binding between nucleic acids through complementary base pairing may be referred to as ‘hybridisation’. Through binding to its target nucleotide sequence, an antisense nucleic acid may form a nucleic acid complex comprising (i) the antisense nucleic acid and (ii) a target nucleic acid comprising the target nucleotide sequence.

The nucleotide sequence of an antisense nucleic acid is sufficiently complementary to its target nucleotide sequence such that it binds or hybridises to the target nucleotide sequence. It will be appreciated that an antisense nucleic acid preferably has a high degree of sequence identity to the reverse complement of its target nucleotide sequence. In some embodiments, the antisense nucleic acid comprises or consists of a nucleotide sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to the reverse complement of its target nucleotide sequence.

In some embodiments, an antisense nucleic acid according to the present disclosure comprises: a nucleotide sequence which is the reverse complement of its target nucleotide sequence, or a nucleotide sequence comprising 1 to 10 (e.g. one of 1, 2, 3, 4, 5, 6, 7, 8, 9 or 10) substitutions relative to the reverse complement of its target nucleotide sequence.

In some embodiments, the target nucleotide sequence for an antisense nucleic acid according to the present disclosure comprises, or consists of, 5 to 100 nucleotides, e.g. one of 10 to 80, 12 to 50, or 15 to 30 nucleotides (e.g. 20 to 27, e.g. ˜21). In some embodiments, the target nucleotide sequence for an antisense nucleic acid according to the present disclosure comprises or consists of DNA and/or RNA. In some embodiments, the target nucleotide sequence for an antisense nucleic acid according to the present disclosure comprises or consists of RNA.

In some embodiments, the antisense nucleic acid reduces/prevents transcription of nucleic acid comprising its target nucleotide sequence. In some embodiments, the antisense nucleic acid reduces/prevents association of factors required for normal transcription (e.g. enhancers, RNA polymerase) with nucleic acid comprising its target nucleotide sequence.

In some embodiments, the antisense nucleic acid increases/potentiates degradation of nucleic acid comprising its target nucleotide sequence, e.g. through RNA interference. In some embodiments, the antisense nucleic acid reduces/prevents translation of nucleic acid comprising its target nucleotide sequence, e.g. through RNA interference or antisense degradation via RNase H activity.

RNA interference is described e.g. in Agrawal et al., Microbiol. Mol. Bio. Rev. (2003) 67(4): 657-685 and Hu et al., Sig. Transduc. Tar. Ther. (2020) 5(101), both of which are hereby incorporated by reference in their entirety. Briefly, double-stranded RNA molecules are recognised by the argonaute component of the RNA-induced silencing complex (RISC). The double-stranded RNAs are separated into single strands and integrated into an active RISC, by the RISC-Loading Complex (RLC). The RISC-integrated strands bind to their target RNA through complementary base pairing, and depending on the identity of the RISC-integrated RNA and degree of complementarity to the target RNA, the RISC then either cleaves the target RNA resulting in its degradation, or otherwise blocks access of ribosomes thereby preventing its translation. RNAi based therapeutics have been approved for a number of indications (see e.g. Kim, Chonnam Med J. (2020) 56(2): 87-93).

In some embodiments, the antisense nucleic acid reduces/prevents normal post-transcriptional processing (e.g. splicing and/or translation) of nucleic acid comprising its target nucleotide sequence. In some embodiments, the antisense nucleic acid reduces or alters splicing of pre-mRNA comprising its target nucleotide sequence to mature mRNA. In some embodiments, the antisense nucleic acid reduces translation of mRNA comprising its target nucleotide sequence to protein.

In some embodiments, the antisense nucleic acid reduces/prevents association of factors required for normal post-transcriptional processing (e.g. components of the spliceosome) with nucleic acid comprising its target nucleotide sequence. In such instances, the antisense nucleic may be referred to as a ‘splice-switching’ nucleic acid.

Splice-switching nucleic acids are reviewed e.g. in Haves and Hastings, Nucleic Acids Res. (2016) 44(14): 6549-6563, which is hereby incorporated by reference in its entirety. Splice-switching nucleic acids include e.g. splice-switching oligonucleotides (SSOs). They disrupt the normal splicing of target RNA transcripts by blocking the RNA:RNA base-pairing and/or protein:RNA binding interactions that occur between components of the splicing machinery and pre-mRNA. Splice-switching nucleic acids may be employed to alter the number/proportion of mature mRNA transcripts encoding ADAMTS14. Splice-switching nucleic acids may be designed to target a specific region of the target transcript, e.g. to effect skipping of exon(s) of interest, e.g. exons encoding domains/regions of interest. SSOs often comprise alterations to oligonucleotide sugar-phosphate backbones in order to reduce/prevent RNAse H degradation, such as e.g. phosphorothioate linkages, phosphorodiamidate linkages such as phosphorodiamidate morpholino (PMOs), and may comprise e.g. peptide nucleic acids (PNAs), locked nucleic acids (LNAs), methoxyethyl nucleotide modifications, e.g. 2′O-methyl (2′OMe) and 2′-O-methoxyethyl (MOE) ribose modifications and/or 5′-methylcytosine modifications.

In some embodiments, the antisense nucleic acid inhibits/reduces translation of nucleic acid comprising its target nucleotide sequence. In some embodiments, the antisense nucleic acid reduces/prevents association of factors required for translation (e.g. ribosomes) with nucleic acid comprising its target nucleotide sequence.

It will be appreciated that the target nucleotide sequence to which an antisense nucleic acid binds is a nucleotide sequence encoding a protein which it is desired to inhibit expression of. Accordingly, in aspects and embodiments of the present disclosure, the target nucleotide sequence for an antisense nucleic acid is a nucleotide sequence of a gene encoding ADAMTS14.

In some embodiments, the target nucleotide sequence is a nucleotide sequence of RNA encoded by a gene encoding ADAMTS14. In some embodiments, the target nucleotide sequence is a nucleotide sequence of RNA encoding ADAMTS14. In some embodiments, the target nucleotide sequence comprises one or more nucleotides of an exon of RNA encoding ADAMTS14. In some embodiments, the target nucleotide sequence is a nucleotide sequence of an exon of RNA encoding ADAMTS14.

In some embodiments, the target nucleotide sequence is a nucleotide sequence of the pre-mRNA transcribed from NCBI Reference Sequence: NG_042147.1 (the genomic sequence encoding human ADAMTS14). In some embodiments, the target nucleotide sequence is a nucleotide sequence of the mature mRNA sequence corresponding to NCBI Reference Sequence: NM_080722.4 (the cDNA sequence encoding human ADAMTS14).

In some embodiments, the target nucleotide sequence is the target nucleotide sequence of an siRNA selected from SASI_Hs01_00087500, SASI_Hs02_00362496, SASI_Hs01_00087503, SASI_Hs02_00362497, SASI_Hs02_00362498, SASI_Hs02_00362499, SASI_Hs02_00362500, SASI_Hs02_00362501, SASI_Hs01_00087507, SASI_Hs02_00362502, SASI_Hs01_00087499, SASI_Hs01_00087501, SASI_Hs01_00087502, SASI_Hs01_00087504, SASI_Hs01_00087505, SASI_Hs01_00087506 and SASI_Hs01_00087508 (Sigma-Aldrich).

In some embodiments, the target nucleotide sequence is the target nucleotide sequence of an shRNA selected from TRCN0000046703, TRCN0000046704, TRCN0000046705, TRCN0000046706, TRCN0000046707, TRCN0000415803, TRCN0000427825 and TRCN0000430560 (Sigma-Aldrich).

In some embodiments, the target nucleotide sequence is the target nucleotide sequence of an siRNA of Smart Pool Cat. No. L-00576-00-0005 (Dharmacon). In some embodiments, the target nucleotide sequence is the target nucleotide sequence of an siRNA of siGENOME Human ADAMTS14 siRNA Cat. No. D-005765-01 (Dharmacon). In some embodiments, the target nucleotide sequence is the target nucleotide sequence of an siRNA of siGENOME Human ADAMTS14 siRNA Cat. No. D-005765-03 (Dharmacon).

In some embodiments, the target nucleotide sequence is the target nucleotide sequence of an miR-29 (e.g. miR-29a, miR-29b or miR-29c). In some embodiments, the target nucleotide sequence is not the target nucleotide sequence of an miR-29 (e.g. miR-29a, miR-29b or miR-29c).

In some embodiments, the antisense nucleic acid comprises, or consists of, a sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to the reverse complement of a nucleotide sequence of the pre-mRNA transcribed from NCBI Reference Sequence: NG_042147.1. In some embodiments, the antisense nucleic acid comprises, or consists of, a sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to the reverse complement of a nucleotide sequence of the mature mRNA sequence corresponding to NCBI Reference Sequence: NM_080722.4.

In some embodiments, the antisense nucleic acid comprises, or consists of, the nucleotide sequence of the guide strand of an siRNA selected from SASI_Hs01_00087500, SASI_Hs02_00362496, SASI_Hs01_00087503, SASI_Hs02_00362497, SASI_Hs02_00362498, SASI_Hs02_00362499, SASI_Hs02_00362500, SASI_Hs02_00362501, SASI_Hs01_00087507, SASI_Hs02_00362502, SASI_Hs01_00087499, SASI_Hs01_00087501, SASI_Hs01_00087502, SASI_Hs01_00087504, SASI_Hs01_00087505, SASI_Hs01_00087506 and SASI_Hs01_00087508 (Sigma-Aldrich).

In some embodiments, the antisense nucleic acid comprises, or consists of, the nucleotide sequence of the guide strand of an siRNA derived from an shRNA selected from TRCN0000046703, TRCN0000046704, TRCN0000046705, TRCN0000046706, TRCN0000046707, TRCN0000415803, TRCN0000427825 and TRCN0000430560 (Sigma-Aldrich).

In some embodiments, the antisense nucleic acid comprises, or consists of, the nucleotide sequence of the guide strand of an siRNA of Smart Pool Cat. No. L-00576-00-0005 (Dharmacon). In some embodiments, the antisense nucleic acid comprises, or consists of, the nucleotide sequence of the guide strand of an siRNA of siGENOME Human ADAMTS14 siRNA Cat. No. D-005765-01 (Dharmacon). In some embodiments, the antisense nucleic acid comprises, or consists of, the nucleotide sequence of the guide strand of an siRNA of siGENOME Human ADAMTS14 siRNA Cat. No. D-005765-03 (Dharmacon).

In some embodiments, the antisense nucleic acid comprises, or consists of, the nucleotide sequence of the guide strand of an miR-29 (e.g. miR-29a, miR-29b or miR-29c). In some embodiments, the antisense nucleic acid does not comprise, or does not consist of, the nucleotide sequence of the guide strand of an miR-29 (e.g. miR-29a, miR-29b or miR-29c).

In some embodiments, an inhibitory nucleic acid is selected from: an siRNA, dsiRNA, miRNA, shRNA, pri-miRNA, pre-miRNA, saRNA, snoRNA, or antisense oligonucleotide (e.g. a gapmer), or a nucleic acid encoding the same. In some embodiments, an inhibitory nucleic acid is selected from: an siRNA, dsiRNA, miRNA, shRNA. In some embodiments, an inhibitory nucleic acid is an siRNA.

In some embodiments, an inhibitory nucleic acid may comprise an antisense nucleic acid described herein, e.g. as part of a larger nucleic acid species. For example, in some embodiments, an inhibitory nucleic acid may be an siRNA, dsiRNA, miRNA, shRNA, pri-miRNA, pre-miRNA, saRNA or snoRNA comprising an antisense nucleic acid described herein.

In some embodiments, an inhibitory nucleic acid is a small interfering RNA (siRNA). As used herein, ‘siRNA’ refers to a double-stranded RNA molecule having a length between 17 to 30 (e.g. 20 to 27, e.g. ˜21) base pairs, which is capable of engaging the RNA interference (RNAi) pathway for the targeted degradation of target RNA. Double-stranded siRNA molecules may be formed as a nucleic acid complex of RNA strands having a high degree of complementarity. In some embodiments, siRNA molecules comprise symmetric 3′ overhangs, e.g. comprising one or two nucleotides (e.g. a ‘UU’ 3′ overhang). The strand of the double-stranded siRNA molecule having complementarity to a target nucleotide sequence (i.e. the antisense nucleic acid) may be referred to as the ‘guide’ strand, and the other strand may be referred to as the ‘passenger’ strand. The structure and function of siRNAs is described e.g. in Kim and Rossi, Biotechniques. (2008) 44(5): 613-616.

In some embodiments, the guide strand of an siRNA according to the present disclosure may comprise or consist of an antisense nucleic acid according to an embodiment of an antisense nucleic acid described herein.

In some embodiments, an inhibitory nucleic acid comprises the guide and passenger strands of an siRNA selected from SASI_Hs01_00087500, SASI_Hs02_00362496, SASI_Hs01_00087503, SASI_Hs02_00362497, SASI_Hs02_00362498, SASI_Hs02_00362499, SASI_Hs02_00362500, SASI_Hs02_00362501, SASI_Hs01_00087507, SASI_Hs02_00362502, SASI_Hs01_00087499, SASI_Hs01_00087501, SASI_Hs01_00087502, SASI_Hs01_00087504, SASI_Hs01_00087505, SASI_Hs01_00087506 and SASI_Hs01_00087508 (Sigma-Aldrich).

In some embodiments, inhibitory nucleic acid comprises the guide and passenger strands of an siRNA derived from an shRNA selected from TRCN0000046703, TRCN0000046704, TRCN0000046705, TRCN0000046706, TRCN0000046707, TRCN0000415803, TRCN0000427825 and TRCN0000430560 (Sigma-Aldrich).

In some embodiments, an inhibitory nucleic acid comprises the guide and passenger strands of an siRNA of Smart Pool Cat. No. L-00576-00-0005 (Dharmacon). In some embodiments, an inhibitory nucleic acid comprises the guide and passenger strands of an siRNA of siGENOME Human ADAMTS14 siRNA Cat. No. D-005765-01 (Dharmacon). In some embodiments, an inhibitory nucleic acid comprises the guide and passenger strands of an siRNA of siGENOME Human ADAMTS14 siRNA Cat. No. D-005765-03 (Dharmacon).

In some embodiments, an inhibitory nucleic acid comprises the guide and passenger strands of an miR-29 (e.g. miR-29a, miR-29b or miR-29c). In some embodiments, an inhibitory nucleic acid does not comprise the guide and passenger strands of an miR-29 (e.g. miR-29a, miR-29b or miR-29c).

In some embodiments, an inhibitory nucleic acid is an siRNA selected from SASI_Hs01_00087500, SASI_Hs02_00362496, SASI_Hs01_00087503, SASI_Hs02_00362497, SASI_Hs02_00362498, SASI_Hs02_00362499, SASI_Hs02_00362500, SASI_Hs02_00362501, SASI_Hs01_00087507, SASI_Hs02_00362502, SASI_Hs01_00087499, SASI_Hs01_00087501, SASI_Hs01_00087502, SASI_Hs01_00087504, SASI_Hs01_00087505, SASI_Hs01_00087506 and SASI_Hs01_00087508 (Sigma-Aldrich).

In some embodiments, inhibitory nucleic acid is an shRNA selected from TRCN0000046703, TRCN0000046704, TRCN0000046705, TRCN0000046706, TRCN0000046707, TRCN0000415803, TRCN0000427825 and TRCN0000430560 (Sigma-Aldrich).

In some embodiments, an inhibitory nucleic acid is an siRNA of Smart Pool Cat. No. L-00576-00-0005 (Dharmacon). In some embodiments, an inhibitory nucleic acid is an siRNA of siGENOME Human ADAMTS14 siRNA Cat. No. D-005765-01 (Dharmacon). In some embodiments, an inhibitory nucleic acid is an siRNA of siGENOME Human ADAMTS14 siRNA Cat. No. D-005765-03 (Dharmacon).

In some embodiments, an inhibitory nucleic acid is an miR-29 (e.g. miR-29a, miR-29b or miR-29c). In some embodiments, an inhibitory nucleic acid is not an miR-29 (e.g. miR-29a, miR-29b or miR-29c).

In some embodiments, an inhibitory nucleic acid is a dicer small interfering RNA (dsiRNA). As used herein, ‘dsiRNA’ refers to a double-stranded RNA molecule having a length of ˜27 base pairs, which is processed by Dicer to siRNA for RNAi-mediated degradation of target RNA. DsiRNAs are described e.g. in Raja et al., Asian J Pharm Sci. (2019) 14(5): 497-510, which is hereby incorporated by reference in their entirety. DsiRNAs are optimised for Dicer processing and may have increased potency compared with 21-mer siRNAs (see e.g. Kim et al., Nat Biotechnol. (2005) 23(2):222-226), which may be related to the link between Dicer-mediated nuclease activity and RISC loading.

In some embodiments, an inhibitory nucleic acid is a micro RNA (miRNA), or a precursor thereof (e.g. a pri-miRNA or a pre-miRNA). miRNA molecules have a similar structure to siRNA molecules, but are encoded endogenously, and derived from processing of short hairpin RNA molecules. They are initially expressed as long primary transcripts (pri-miRNAs), which are processed within the nucleus into 60 to 70 nucleotide hairpins (pre-miRNAs), which are further processed in the cytoplasm into smaller species that interact with RISC and target mRNA. miRNAs comprise ‘seed sequences’ that are essential for binding to target mRNA. Seed sequences usually comprise six nucleotides and are situated at positions 2 to 7 at the miRNA 5′ end.

Cushing et al., miRNA Biochem Cell Biol. (2015) 93(2):109-18 is a review article relating to an miRNA designated miR-29, disclosing that miR-29 is an inhibitor of the expression of a wide range of genes implicated in extracellular matrix production, crosslinking and degradation, genes involved in basement membrane function, PDGF genes, and interleukin and TGF genes (see e.g. FIG. 1). ADAMTS14 is identified as a gene whose expression is repressed by miR-29.

In some embodiments, an ADAMTS14 inhibitor according to the present disclosure is an miR-29 (e.g. miR-29a, miR-29b or miR-29c). In some embodiments, an ADAMTS14 inhibitor according to the present disclosure is not an miR-29 (e.g. miR-29a, miR-29b or miR-29c); that is, in some embodiments, an ADAMTS14 inhibitor according to the present disclosure is an ADAMTS14 inhibitor other than an miR-29 (e.g. miR-29a, miR-29b or miR-29c).

In some embodiments, an inhibitory nucleic acid is a short hairpin RNA (shRNA). shRNA molecules comprise sequences of nucleotides having a high degree of complementarity that associate with one another through complementary base pairing to form the stem region of the hairpin. The sequences of nucleotides having a high degree of complementarity may be linked by one or more nucleotides that form the loop region of the hairpin. shRNA molecules may be processed (e.g. via catalytic cleavage by DICER) to form siRNA or miRNA molecules. shRNA molecules may have a length of between 35 to 100 (e.g. 40 to 70) nucleotides. The stem region of the hairpin may have a length between 17 to 30 (e.g. 20 to 27, e.g. ˜21) base pairs. The stem region may comprise G-U pairings to stabilise the hairpin structure.

siRNA, dsiRNA, miRNAs and shRNAs for the targeted inhibition of gene and/or protein expression of ADAMTS14 may be identified/designed in accordance with principles and/or using tools well known to the skilled person. Parameters and tools for designing siRNA and shRNA molecules are described e.g. in Fakhr et al., Cancer Gene Therapy (2016) 23:73-82 (hereby incorporated by reference in its entirety). Software that may be used by the skilled person for the design of such molecules is summarised in Table 1 of Fakhr et al., Cancer Gene Therapy (2016) 23:73-82, and includes e.g. siRNA Wizard (InvivoGen). Details for designing and making such molecules can be found on the websites of commercial vendors such as Ambion, Dharmacon, GenScript, Invitrogen and OligoEngine.

In some embodiments, an inhibitory nucleic acid is an antisense oligonucleotide (ASO). ASOs are single-stranded nucleic acid molecules comprising or consisting of an antisense nucleic acid to a target nucleotide sequence. An antisense oligonucleotide according to the present disclosure may comprise or consist of an antisense nucleic acid as described herein.

ASOs can modify expression of RNA molecules comprising their target nucleotide sequence by altering splicing, or by recruiting RNase H to degrade RNA comprising the target nucleotide sequence. RNase H recognises nucleic acid complex molecules formed when the ASO binds to RNA comprising its target nucleotide sequence. ASOs according to the present disclosure may comprise or consist of an antisense nucleic acid according to the present disclosure. ASOs may comprise 17 to 30 (e.g. 20 to 27, e.g. ˜21) nucleotides in length. Many ASOs are designed as chimeras, comprising a mix of bases with different chemistries, or as gapmers, comprising a central DNA portion surrounded by ‘wings’ of modified nucleotides. ASOs are described in e.g. Scoles et al., Neurol Genet. 2019 April; 5(2): e323. ASOs sometimes comprise alterations to the sugar-phosphate backbone in order to increase their stability and/or reduce/prevent RNAse H degradation, such as e.g. phosphorothioate linkages, phosphorodiamidate linkages such as phosphorodiamidate morpholino (PMOs), and may comprise e.g. peptide nucleic acids (PNAs), locked nucleic acids (LNAs), methoxyethyl nucleotide modifications, e.g. 2′O-methyl (2′OMe) and 2′-O-methoxyethyl (MOE) ribose modifications and/or 5′-methylcytosine modifications.

Inhibitory nucleic acids according to the present disclosure may comprise chemically modified nucleotides, e.g. in which the phosphonate and/or ribose and/or base is/are chemically modified. Such modifications may influence the activity, specificity and/or stability of nucleic acid. One or more (e.g. one of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or all) nucleotides of an inhibitory nucleic acid may comprise such chemical modification.

Modifications contemplated in accordance with inhibitory nucleic acids of the present disclosure include those described in Hu et al., Sig. Transduc. Tar. Ther. (2020) 5(101) (incorporated by reference hereinabove), in particular those shown in FIG. 2 of Hu et al., Sig. Transduc. Tar. Ther. (2020) 5(101). Further modifications contemplated in accordance with inhibitory nucleic acids according to the present disclosure include those described in Selvam et al., Chem Biol Drug Des. (2017) 90(5): 665-678, which is hereby incorporated by reference in its entirety).

In some embodiments, an inhibitory nucleic acid according to the present disclosure comprises one or more nucleotides comprising a phosphonate modification. In some embodiments, the phosphonate modification(s) may be selected from: phosphorothioate (e.g. Rp isomer, Sp isomer), phosphorodithioate, methylphosphonate, methoxypropylphosphonate, 5′-(E)-vinylphosphonate, 5′-methylphosphonate, (S)-5′-C-methyl with phosphate, 5′-phosphorothioate, and peptide nucleic acid. In some embodiments, an inhibitory nucleic acid comprises one or more nucleotides comprising phosphorothioate modification.

In some embodiments, an inhibitory nucleic acid according to the present disclosure comprises one or more nucleotides comprising a ribose modification. In some embodiments, the ribose modification(s) may be selected from: 2′-O-methyl, 2′-O-methoxyethyl, 2′-fluoro, 2′-deoxy-2′-fluoro, 2′-methoxyethyl, 2′-O-alkyl, 2′-O-allyl, 2′-C-allyl, 2′-deoxy, 2′-hydroxyl, 2′-arabino-fluoro, 2′-O-benzyl, 2′-O-methyl-4-pyridine, locked nucleic acid, (S)-cEt-BNA, tricyclo-DNA, PMO, unlocked nucleic acid, hexitol nucleic acid and glycol nucleic acid. In some embodiments, an inhibitory nucleic acid comprises one or more nucleotides comprising 2′-O-methyl modification. In some embodiments, an inhibitory nucleic acid comprises one or more nucleotides comprising 2′-fluoro modification.

In some embodiments, an inhibitory nucleic acid according to the present disclosure comprises one or more nucleotides comprising a base modification. In some embodiments, the base modification(s) may be selected from: pseudouridine, 2′-thiouridine, N6′-methyladenosine, 5′-methylcytidine, 5′-fluoro-2′-deoxyuridine, N-ethylpiperidine 7′-EAA triazole-modified adenine, N-ethylpiperidine 6′-triazole-modified adenine, 6′-phenylpyrrolo-cytosine, 2′,4′-difluorotoluyl ribonucleoside and 5′-nitroindole.

In some embodiments, an inhibitory nucleic acid according to the present disclosure comprises: one or more nucleotides comprising phosphorothioate modification, one or more nucleotides comprising 2′-O-methyl modification, and one or more nucleotides comprising 2′-fluoro modification.

In embodiments wherein inhibitory nucleic acids comprise nucleotides comprising chemical modification as described herein, the nucleotide sequence is nevertheless evaluated for the purposes of sequence comparison in accordance with the present disclosure as if the equivalent unmodified nucleotide were instead present.

In some embodiments, an inhibitory nucleic acid (e.g. an siRNA) according to the present disclosure may inhibit gene and/or protein expression of ADAMTS14 with an IC₅₀ of ≤1 pM, e.g. one of ≤500 nM, ≤100 nM, ≤75 nM, ≤50 nM, ≤40 nM, ≤30 nM, ≤20 nM, ≤15 nM, ≤12.5 nM, ≤10 nM, ≤9 nM, ≤8 nM, ≤7 nM, ≤6 nM, ≤5 nM, ≤4 nM ≤3 nM, ≤2 nM, ≤1 nM, ≤900 pM, ≤800 pM, ≤700 pM, ≤600 pM, ≤500 pM, ≤400 pM, ≤300 pM, ≤200 pM, ≤100 pM, ≤50 pM, ≤40 pM, ≤30 pM, ≤20 pM, ≤10 pM or ≤1 pM.

In some embodiments an inhibitory nucleic acid according to the present disclosure (e.g. an siRNA) may inhibit gene expression of ADAMTS14 (e.g. as determined by qRT-PCR) with an IC₅₀ of ≤1 nM, ≤900 pM, ≤800 pM, ≤700 pM, ≤600 pM, ≤500 pM, ≤400 pM, ≤300 pM, ≤200 pM, ≤100 pM, ≤50 pM, ≤40 pM, ≤30 pM, ≤20 pM, ≤10 pM or ≤1 pM.

In some embodiments an inhibitory nucleic acid according to the present disclosure (e.g. an siRNA) may inhibit protein expression of ADAMTS14 (e.g. as determined by ELISA) with an IC₅₀ of ≤1 nM, ≤900 pM, ≤800 pM, ≤700 pM, ≤600 pM, ≤500 pM, ≤400 pM, ≤300 pM, ≤200 pM, ≤100 pM, ≤50 pM, ≤40 pM, ≤30 pM, ≤20 pM, ≤10 pM or ≤1 pM.

Site-specific nuclease (SSN) systems may be employed to reduce gene and/or protein expression of ADAMTS14. In accordance with such embodiments, the reduction in gene and/or protein expression can be achieved by employing the SSN system modifying nucleic acid encoding ADAMTS14.

The SSN system may be capable of modifying a gene encoding ADAMTS14. The SSN system may be capable of introducing an insertion, substitution or deletion into a nucleic acid sequence encoding ADAMTS14. Modification to nucleic acid sequence encoding ADAMTS14 by the SSN system may reduce or prevent expression of a polypeptide according to SEQ ID NO:1, 2, 3 or 4 from the modified nucleic acid sequence. Modification to nucleic acid sequence encoding ADAMTS14 by the SSN system in a cell may have the result that the cell lacks nucleic acid encoding a polypeptide according to SEQ ID NO:1, 2, 3, or 4.

The SSN system may be capable of introducing a premature stop codon in the sequence transcribed from ADAMTS14. Modification to nucleic acid sequence encoding ADAMTS14 by the SSN system may have the result that the modified nucleic acid encodes a truncated and/or non-functional ADAMTS14 polypeptide, and/or an ADAMTS14 polypeptide that is misfolded and/or degraded.

Gene editing using SSNs is reviewed e.g. in Eid and Mahfouz, Exp Mol Med. 2016 October; 48(10): e265, which is hereby incorporated by reference in its entirety. Enzymes capable of creating site-specific double strand breaks (DSBs) can be engineered to introduce DSBs to target nucleic acid sequence(s) of interest. DSBs may be repaired by either error-prone non-homologous end-joining (NHEJ), in which the two ends of the break are rejoined, often with insertion or deletion of nucleotides. Alternatively, DSBs may be repaired by highly homology-directed repair (HDR), in which a DNA template with ends homologous to the break site is supplied and introduced at the site of the DSB.

SSNs capable of being engineered to generate target nucleic acid sequence-specific DSBs include zinc-finger nucleases (ZFNs), transcription activator-like effector nucleases (TALENs) and clustered regularly interspaced palindromic repeats/CRISPR-associated-9 (CRISPR/Cas9) systems.

ZFN systems are reviewed e.g. in Umov et al., Nat Rev Genet. (2010) 11(9):636-46, which is hereby incorporated by reference in its entirety. ZFNs comprise a programmable Zinc Finger DNA-binding domain and a DNA-cleaving domain (e.g. a Fokl endonuclease domain). The DNA-binding domain may be identified by screening a Zinc Finger array capable of binding to the target nucleic acid sequence.

TALEN systems are reviewed e.g. in Mahfouz et al., Plant Biotechnol J. (2014) 12(8):1006-14, which is hereby incorporated by reference in its entirety. TALENs comprise a programmable DNA-binding TALE domain and a DNA-cleaving domain (e.g. a Fokl endonuclease domain). TALEs comprise repeat domains consisting of repeats of 33-39 amino acids, which are identical except for two residues at positions 12 and 13 of each repeat which are repeat variable di-residues (RVDs). Each RVD determines binding of the repeat to a nucleotide in the target DNA sequence according to the following relationship: ‘HD’ binds to C, ‘NI’ binds to A, ‘NG’ binds to T and ‘NN’ or ‘NK’ binds to G (Moscou and Bogdanove, Science (2009) 326(5959):1501.).

CRISPR/Cas9 and related systems e.g. CRISPR/Cpf1, CRISPR/C2c1, CRISPR/C2c2 and CRISPR/C2c3 are reviewed e.g. in Nakade et al., Bioengineered (2017) 8(3):265-273, which is hereby incorporated by reference in its entirety. These systems comprise an endonuclease (e.g. Cas9, Cpf1 etc.) and the single-guide RNA (sgRNA) molecule. The sgRNA can be engineered to target endonuclease activity to nucleic acid sequences of interest.

In some embodiments, the SSN system for reducing expression of ADAMTS14 according to the present disclosure is selected from: a ZFN system, a TALEN system, CRISPR/Cas9 system, a CRISPR/Cpf1 system, a CRISPR/C2c1 system, a CRISPR/C2c2 system, and a CRISPR/C2c3 system.

For example, the SSN system may employ nucleic acid(s) encoding a CRISPR/Cas9 system. The nucleic acid(s) may encode a CRISPR RNA (crRNA) targeting an exon of ADAMTS14 and a trans-activating crRNA (tracrRNA) for processing the crRNA to its mature form.

ADAMTS14 inhibitors according to the present disclosure may be provided in the form of nucleic acid comprising/encoding the ADAMTS14 inhibitors. By way of illustration, peptides/polypeptides capable of binding to and inhibiting the activity of ADAMTS14 and SSN systems for reducing expression of ADAMTS14 may be provided in the form of nucleic acid(s) encoding the same. Likewise, nucleic acids capable of binding to and inhibiting the activity of ADAMTS14 and inhibitory nucleic acids capable of reducing expression of ADAMTS14 may be provided in the form of nucleic acid(s) comprising/encoding the same.

Nucleic acids comprising/encoding ADAMTS14 inhibitors according to the present disclosure may comprise, or consist of, DNA and/or RNA. In some embodiments, nucleic acids comprising/encoding ADAMTS14 inhibitors according to the present disclosure may be contained in a vector. The vector may facilitate delivery of the nucleic acid(s) comprising/encoding an ADAMTS14. The vector may be an expression vector comprising elements required for expressing nucleic acid(s) comprising/encoding an ADAMTS14 inhibitor.

A ‘vector’ as used herein is a nucleic acid molecule used as a vehicle to transfer exogenous nucleic acid into a cell. The vector may be a vector for expression of the nucleic acid in the cell. Such vectors may include a promoter sequence operably linked to the nucleotide sequence encoding the sequence to be expressed. A vector may also include a termination codon and expression enhancers. Any suitable vectors, promoters, enhancers and termination codons known in the art may be used to express nucleic acid from a vector according to the present disclosure.

The term ‘operably linked’ may include the situation where a selected nucleic acid sequence and regulatory nucleic acid sequence (e.g. promoter and/or enhancer) are covalently linked in such a way as to place the expression of nucleic acid sequence under the influence or control of the regulatory sequence (thereby forming an expression cassette). Thus, a regulatory sequence is operably linked to the selected nucleic acid sequence if the regulatory sequence is capable of affecting transcription of the nucleic acid sequence.

Suitable vectors include plasmids, binary vectors, DNA vectors, mRNA vectors, viral vectors (e.g. gammaretroviral vectors (e.g. murine Leukemia virus (MLV)-derived vectors), lentiviral vectors, adenovirus vectors, adeno-associated virus vectors, lentivirus vectors, vaccinia virus vectors and herpesvirus vectors), transposon-based vectors, and artificial chromosomes (e.g. yeast artificial chromosomes), e.g. as described in Maus et al., Annu Rev Immunol (2014) 32:189-225 or Morgan and Boyerinas, Biomedicines 2016 4, 9, which are both hereby incorporated by reference in their entirety. In preferred embodiments, the vector is an adeno-associated virus (AAV) vector or a lentiviral vector.

In some embodiments, the vector may be a eukaryotic vector, e.g. a vector comprising the elements necessary for expression of nucleic acid from the vector in a eukaryotic cell. In some embodiments, the vector may be a mammalian vector, e.g. comprising a cytomegalovirus (CMV) or SV40 promoter to drive expression. In some embodiments, the vector comprises a cell- or tissue-specific promoter. In some embodiments, the vector comprises a fibroblast-specific promoter.

In some embodiments, a vector is selected based on tropism for a cell type/tissue/organ to which it is desired to deliver the nucleic acid. In some embodiments, a vector is selected based on tropism for a cell type/tissue/organ in which it is desired to express an ADAMTS14 inhibitor. For example, it may be desired to deliver the nucleic acid/express the an ADAMTS14 inhibitor in a cell type/tissue/organ affected by a disease to be treated/prevented in accordance with the present disclosure (e.g. a cell type/tissue/organ in which the symptoms of the disease manifest).

In preferred embodiments, the vector is an adeno-associated virus vector. Adeno-associated virus vectors and their use to vector gene therapy is reviewed e.g. in Wang et al., Nat. Rev. Drug Discov. (2019) 18: 358-378 and Li and Samulski, Nat. Rev. Genet. (2020) 12: 255-272, both of which are hereby incorporated by reference in their entirety. In some embodiments, a vector may be an adeno-associated virus vector described in Wang et al., Nat. Rev. Drug Discov. (2019) 18: 358-378. In some embodiments, a vector may be an adeno-associated virus vector described in Li and Samulski, Nat. Rev. Genet. (2020) 12: 255-272.

A peptide/polypeptide capable of binding to and inhibiting the activity of ADAMTS14 according to the present disclosure may be produced within a cell by transcription from a vector encoding the peptide/polypeptide, and subsequent translation of the transcribed RNA.

A nucleic acid aptamer capable of binding to and inhibiting the activity of ADAMTS14 may be produced within a cell by transcription from a vector encoding the nucleic acid aptamer. An shRNA molecule according to the present disclosure may be produced within a cell by transcription from a vector encoding the shRNA. shRNAs may be produced within a cell by transfecting the cell with a vector encoding the shRNA sequence under control of an RNA polymerase promoter. An siRNA molecule according to the present disclosure may be produced within a cell by transcription from a vector encoding shRNA encoding/comprising the siRNA, and subsequent processing of the shRNA molecule by cellular DICER to form the siRNA molecule.

ADAMTS14 inhibitors according to the present disclosure may be provided in the form of cells comprising nucleic acid comprising/encoding the ADAMTS14 inhibitors, which may e.g. be contained in a vector as described hereinabove.

The articles of the present disclosure (i.e. the ADAMTS14 inhibitors, nucleic acids comprising/encoding ADAMTS14 inhibitors, vectors comprising such nucleic acids, cells comprising such nucleic acids or vectors) may be formulated as compositions suitable for clinical use (i.e. as medicaments or pharmaceutical compositions).

Such compositions may comprise one or more pharmaceutically-acceptable ingredients well known to those skilled in the art. Such ingredients include, but are not limited to, pharmaceutically-acceptable carriers, adjuvants, excipients, diluents, fillers, buffers, preservatives, anti-oxidants, lubricants, stabilisers, solubilisers, surfactants (e.g., wetting agents), masking agents, colouring agents, flavouring agents, and sweetening agents.

The term ‘pharmaceutically-acceptable’ as used herein pertains to compounds, ingredients, materials, compositions, dosage forms, etc., which are, within the scope of sound medical judgment, suitable for use in contact with the tissues of the subject in question (e.g., human) without excessive toxicity, irritation, allergic response, or other problem or complication, commensurate with a reasonable benefit/risk ratio.

Each carrier, adjuvant, excipient, etc. must also be ‘acceptable’ in the sense of being compatible with the other ingredients of the formulation. Suitable carriers, adjuvants, excipients, etc. can be found in standard pharmaceutical texts, for example, Remington's ‘The Science and Practice of Pharmacy’ (ed. A. Adejare), 23rd Edition (2020), Academic Press.

The compositions may be prepared for topical, parenteral, systemic, intracavitary, intravenous, intra-arterial, intramuscular, intrathecal, intraocular, intravitreal, intraconjunctival, subretinal, suprachoroidal, subcutaneous, intradermal, intrathecal, oral, nasal or transdermal routes of administration which may include injection or infusion. Suitable formulations may comprise the selected agent in a sterile or isotonic medium. The formulation and mode of administration may be selected according to the agent to be administered, and disease to be treated/prevented.

Nucleic acids (including inhibitory nucleic acids, expression vectors), cells and compositions according to the present disclosure may be modified and/or formulated to facilitate delivery to, and/or uptake by, a cell/tissue of interest, e.g. a fibroblast.

Strategies for targeted delivery of such species are reviewed e.g. in Li et al., Int. J. Mol. Sci. (2015) 16: 19518-19536 and Fu et al., Bioconjug Chem. (2014) 25(9): 1602-1608, which are hereby incorporated by reference in their entirety. In particular, nucleic acids according to the present disclosure may employ a delivery platform described in Hu et al., Sig. Transduc. Tar. Ther. (2020) 5(101) (incorporated by reference hereinabove), or Tatiparti et al. ‘siRNA Delivery Strategies: A Comprehensive Review of Recent Developments.’ Ed. Thomas Nann. Nanomaterials 7.4 (2017): 77, and Lehto T et al., Adv Drug Deliv Rev. 2016, 106(Pt A):172-182, which are hereby incorporated by reference in their entirety.

In some embodiments, articles of the present disclosure may be encapsulated in a nanoparticle or a liposome. In some embodiments, articles of the present disclosure may be (covalently or non-covalently) associated with a cell-penetrating peptide (e.g. a protein transduction domain, trojan peptide, arginine-rich peptide, vectocell peptide), a cationic polymer, a cationic lipid or a viral carrier.

Nanoparticles may be organic, e.g. micelles, liposomes, proteins, solid-lipid particles, solid polymer particles, dendrimers, and polymer therapeutics. Nanoparticles may be inorganic, e.g. such as nanotubes or metal particles, optionally with organic molecules added. In some embodiments, a nanoparticle is a nanoparticle described in Chen et al., Mol Ther Methods Clin Dev. (2016) 3:16023, which is hereby incorporated by reference in its entirety. In some embodiments, a nanoparticle is a PLGA, polypeptide, poly(β-amino ester), DOPE, β-cyclodextrin-containing polycation, linear PEI, PAMAM dendrimer, branched PEI, chitosan or polyphosophoester nanoparticle.

In some embodiments, nucleic acids according to the present disclosure comprise modification to incorporate one or more moieties facilitating delivery to, and/or uptake by, a cell type or tissue of interest. In some embodiments, nucleic acids according to the present disclosure are linked (e.g. chemically conjugated to) one or more moieties facilitating delivery to, and/or uptake by, a cell type or tissue of interest.

Modification to, and formulation of, nucleic acids to facilitate targeted delivery to cell types and/or tissues of interest is described e.g. in Lorenzer et al., J Control Release (2015) 203:1-15, which is hereby incorporated by reference in its entirety. The moiety facilitating delivery to, and/or uptake by, a cell type or tissue of interest may bind selectively to the target cell type/tissue of interest. The moiety may facilitate traversal of the cell membrane of the target cell type and/or of cells of the tissue of interest. The moiety may bind to a molecule expressed at the cell surface of the target cell type/tissue of interest. The moiety may facilitate internalisation of the nucleic acid by the target cell type/tissue of interest (e.g. by endocytosis).

Moieties facilitating delivery to, and/or uptake by, cell types or tissues of interest are described e.g. in Benizri et al., Bioconjug Chem. (2019) 30(2): 366-383, which is hereby incorporated by reference in its entirety. Such moieties include e.g. N-acetylgalactosamine (GalNAc), α-tocopherol, cell-penetrating peptides, nucleic acid aptamers, antibodies and antigen-binding fragments/derivatives thereof, cholesterol, squalene, polyethylene glycol (PEG), fatty acids (e.g. palmitic acid) and nucleolipid moieties.

### Pro-Fibrotic Fibroblast Inhibition

Aspects and embodiments of the present disclosure are concerned with the use of ADAMTS14 inhibitors to inhibit the generation of, and/or processes mediated by, pro-fibrotic fibroblasts (e.g. myofibroblasts).

Fibroblasts are the most common cell type found in connective tissue. The development and biology of fibroblasts is described e.g. in Plikus et al. Cell (2021) 184(15):3852-3872 (hereby incorporated by reference in its entirety), and the role of fibroblasts in fibrosis is described e.g. in Kendall and Feghali-Bostwick, Front. Pharmacol. (2014) 5:123 (hereby incorporated by reference in its entirety).

As referred to herein, ‘pro-fibrotic fibroblasts’ are fibroblasts which contribute positively to tissue/organ fibrosis through production (i.e. secretion, deposition) of extracellular matrix (ECM) components (e.g. collagen).

Pro-fibrotic fibroblasts include myofibroblasts. Myofibroblasts are described e.g. in Baum and Duffy, J Cardiovasc Pharmacol. (2011) 57(4): 376-379 and Bagalad et al., J Oral Maxillofac Pathol. (2017) 21(3): 462-463, both of which are hereby incorporated by reference in their entirety.

Myofibroblasts are contractile cells having stress fibers comprising a smooth muscle actin (αSMA). Myofibroblasts may be further characterised by the expression of: type 1, 2 and 3 collagens, fibronectin and/or MMP-9. Myofibroblasts may also be characterised by the production of extracellular matrix comprising fibronectin (e.g. EDA isoform of fibronectin), collagens (e.g. type 1 collagens, e.g. a1 type 1 collagen and a2 type 1 collagen), and hyaluronic acid. Further markers of myofibroblasts include vimentin and palladin.

Myofibroblasts can derive from a wide variety of different cell types. Myofibroblasts can develop by differentiation from tissue-resident fibroblasts in response to proinflammatory mediators such as TGFβ1, IL-4, IL-6, IL-13, ET-1, IGF-II and PDGF, undergoing a fibroblast-to-myofibroblast transition (FMT). The same factors can also activate stellate cells—e.g. hepatic stellate cells (HSCs) in the liver, and pancreatic stellate cells in the pancreas—to develop into myofibroblasts. Epithelial cells are stimulated to undergo an epithelial-to-mesenchymal cell transition to myofibroblasts in response to proinflammatory factors such as TGFβ1, TNFα, IL-1β and IL-4 (see e.g. Li et al., Exp Biol Med (Maywood). (2016) 241(1): 1-13), and TGFβ1, TNFα and IL-1β can also induce endothelial cells to undergo an endothelial-to-mesenchymal cell transition to myofibroblasts. Myofibroblasts also differentiate from bone marrow-derived fibrocyte precursors in response to factors including CCL21, TGFβ1, IL-4, IL-13, PDGF and CXCL12, and proinflammatory mediators can also include smooth muscle cells to ‘de-differentiate’ into myofibroblasts.

Myofibroblasts are key effectors in fibrosis. Fibrosis is a form of pathologic tissue remodelling characterised by the formation of excess connective tissue as a consequence of the excess deposition of extracellular matrix (ECM) components (including collagen). ‘Excess connective tissue’ refers to an amount of connective tissue at a given location (e.g. a given tissue/organ, or part of a given tissue/organ) which is greater than the amount of connective tissue present at that location under normal, non-pathological conditions. Similarly, ‘excess deposition of ECM components’ refers to a level of deposition of one or more ECM components which is greater than the level of deposition under normal, non-pathological conditions.

The cellular and molecular mechanisms of fibrosis are described in Wynn, J. Pathol. (2008) 214(2): 199-210, and Wynn and Ramalingam, Nature Medicine (2012) 18:1028-1040, both of which are hereby incorporated by reference in their entirety.

Damage to tissues can result from various stimuli, including infections, autoimmune reactions, toxins, radiation and mechanical injury. Repair typically involves replacement of injured cells by cells of the same type, and replacement of normal parenchymal tissue with connective tissue. Repair processes become pathologic when they are not controlled properly, resulting in excess deposition of ECM components in which normal parenchymal tissue is replaced with connective tissue. In diseases such as idiopathic pulmonary fibrosis, liver cirrhosis, cardiovascular fibrosis, systemic sclerosis and nephritis, extensive tissue remodelling and fibrosis can ultimately lead to organ failure and death.

The main cellular effectors of fibrosis are myofibroblasts. In response to tissue injury, damaged cells and leukocytes produce pro-fibroinflammatory factors such as TGFβ, IL-13 and PDGF, which activate fibroblasts (and other myofibroblast precursor cells) to become αSMA-expressing myofibroblasts, and recruit myofibroblasts to the site of injury. Myofibroblasts produce large amounts of extracellular matrix components such as collagen and periostin for wound contracture and closure, and also produce proinflammatory cytokines such as IL-6, and tissue remodelling factors such as MMP2 and TIMP1. Persistent/chronic infection and/or inflammation can result in the generation of too many myofibroblasts, and consequently the over-production of extracellular matrix, resulting in fibrosis. In many diseases and conditions characterised by fibrosis, a persistent inflammatory trigger is crucial to upregulation of production of growth factors, proteolytic enzymes, angiogenic factors and fibrogenic cytokines, which stimulate the deposition of connective tissue elements that progressively remodel and destroy normal tissue architecture.

ADAMTS14 inhibitors are demonstrated in the experimental examples of the present disclosure to inhibit the expression of pro-fibrotic genes in fibroblasts in response to stimulation with pro-fibrotic factors (e.g. TGFβ1)—see FIGS. 1 and 6, and Examples 2.1 and 2.6. Inhibition of ADAMTS14 is also shown to inhibit the formation of αSMA-containing fibers in, and collagen I deposition by, fibroblasts in response to stimulation with such pro-fibrotic factors—see FIG. 5 and Example 2.5.

Thus, the experimental examples of the present disclosure demonstrate that ADAMTS14 inhibitors are useful to inhibit the generation of myofibroblasts, and to inhibit processes mediated by myofibroblasts.

TGFβ1-mediated expression of pro-fibrotic genes has been shown to be a conserved mechanism driving fibrosis across a wide range of tissues, and similarly myofibroblasts have been shown to be central effectors of fibrosis in a diversity of tissues, and in a variety of different fibrotic disorders (see e.g. Kim et al., Cold Spring Harb Perspect Biol. (2018) 10(4):a022293). Thus, demonstration in the present experimental examples that ADAMTS14 inhibitors antagonise TGFβ1-mediated upregulation of the expression of pro-fibrotic genes, and inhibit the generation and pro-fibrotic activity of myofibroblasts indicates that ADAMTS14 inhibitors are useful for the treatment/prevention of fibrosis in general, across all tissues and the full range of fibrotic disorders.

Accordingly, the present disclosure provides an ADAMTS14 inhibitor for use in a method comprising inhibiting the generation of myofibroblasts from myofibroblast precursor cells. Also provided is the use of an ADAMTS14 inhibitor in a method of inhibiting the generation of myofibroblasts from myofibroblast precursor cells. Also provided is a method of inhibiting the generation of myofibroblasts from myofibroblast precursor cells, comprising contacting myofibroblast precursor cells with an ADAMTS14 inhibitor. In some embodiments, myofibroblast precursor cells may be selected from fibroblasts, stellate cells (e.g. hepatic stellate cells, pancreatic stellate cells) epithelial cells, endothelial cells, fibrocytes, and smooth muscle cells.

In some embodiments, a myofibroblast precursor cell is a fibroblast. In some embodiments, the uses and methods comprise inhibition of the fibroblast-to-myofibroblast transition (FMT).

The present disclosure also provides an ADAMTS14 inhibitor for use in a method comprising inhibiting TGFβ-mediated signalling. Also provided is the use of an ADAMTS14 inhibitor in a method of inhibiting TGFβ-mediated signalling. Also provided is a method of inhibiting TGFβ-mediated signalling, comprising contacting cells (e.g. myofibroblasts or myofibroblast precursor cells) with an ADAMTS14 inhibitor.

In some embodiments, the uses and methods comprise inhibition of TGFβ-mediated signalling in a myofibroblast and/or a myofibroblast precursor cell (e.g. a fibroblast).

The present disclosure also provides an ADAMTS14 inhibitor for use in a method comprising inhibiting a process mediated by myofibroblasts. Also provided is the use of an ADAMTS14 inhibitor in a method of inhibiting a process mediated by myofibroblasts. Also provided is a method of inhibiting a process mediated by myofibroblasts, comprising contacting myofibroblasts or myofibroblast precursor cells with an ADAMTS14 inhibitor. In some embodiments, a process mediated by myofibroblasts may be selected from: production/deposition of collagen (e.g. type I collagen), production/deposition of extracellular matrix, fibrosis, and tissue remodelling (e.g. epithelial tissue remodelling).

In accordance with such aspects and embodiments, myofibroblasts and/or myofibroblast precursor cells may be contacted with an ADAMTS14 inhibitor in vitro or in vivo. In some embodiments, an ADAMTS14 inhibitor is introduced into a myofibroblast and/or a myofibroblast precursor cell (e.g. by transfection or transduction).

In some embodiments, an ADAMTS14 inhibitor according to the present disclosure reduces the number/proportion of myofibroblasts to less than 1 times, e.g. ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the number/proportion of such cells observed in the absence of the ADAMTS14 inhibitor, or in the presence of the same quantity of a control agent known not to possess such inhibitory activity agent, in a given assay. In some embodiments, an ADAMTS14 inhibitor according to the present disclosure reduces the number/proportion of myofibroblasts to less than 100%, e.g. one of ≤99%, ≤95%, ≤90%, ≤85%, ≤80%, ≤75%, ≤70%, ≤65%, ≤60%, ≤55%, ≤50%, ≤45%, ≤40%, ≤35%, ≤30%, ≤25%, ≤20%, ≤15%, ≤10%, ≤5%, or ≤1% of number/proportion of such cells observed in the absence of the ADAMTS14 inhibitor, or in the presence of the same quantity of a control agent known not to possess such inhibitory activity, in a given assay.

In some embodiments, an ADAMTS14 inhibitor according to the present disclosure inhibits a process mediated by myofibroblasts to less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the level observed in the absence of the ADAMTS14 inhibitor, or in the presence of the same quantity of a control agent known not to possess such inhibitory activity, in a given assay. In some embodiments, an ADAMTS14 inhibitor according to the present disclosure inhibits a process mediated by myofibroblasts to less than 100%, e.g. one of ≤99%, ≤95%, ≤90%, ≤85%, ≤80%, ≤75%, ≤70%, ≤65%, ≤60%, ≤55%, ≤50%, ≤45%, ≤40%, ≤35%, ≤30%, ≤25%, ≤20%, ≤15%, ≤10%, ≤5%, or ≤1% of the level observed in the absence of the ADAMTS14 inhibitor, or in the presence of the same quantity of a control agent known not to possess such inhibitory activity, in a given assay.

In some embodiments, an ADAMTS14 inhibitor according to the present disclosure reduces production/deposition of collagen (e.g. type I collagen) or production/deposition of extracellular matrix or fibrosis to less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the level observed in the absence of the ADAMTS14 inhibitor, or in the presence of the same quantity of a control agent known not to possess such inhibitory activity, in a given assay. In some embodiments, an ADAMTS14 inhibitor according to the present disclosure reduces production/deposition of collagen (e.g. type I collagen) or production/deposition of extracellular matrix or fibrosis to less than 100%, e.g. one of ≤99%, ≤95%, ≤90%, ≤85%, ≤80%, ≤75%, ≤70%, ≤65%, ≤60%, ≤55%, ≤50%, ≤45%, ≤40%, ≤35%, ≤30%, ≤25%, ≤20%, ≤15%, ≤10%, ≤5%, or ≤1% of the level observed in the absence of the ADAMTS14 inhibitor, or in the presence of the same quantity of a control agent known not to possess such inhibitory activity, in a given assay.

### Therapy and Prophylaxis

The present invention provides methods and articles of the present disclosure for the treatment and/or prevention of diseases and conditions in which myofibroblasts are pathologically-implicated.

Treatment is achieved through inhibition of ADAMTS14, e.g. in myofibroblasts or myofibroblast precursor cells. Inhibition of ADAMTS14 inhibits the generation of myofibroblasts from myofibroblast precursor cells, and/or the activity of myofibroblasts.

Accordingly, the present disclosure provides an ADAMTS14 inhibitor for use in the treatment or prevention of a disease/condition in which myofibroblasts are pathologically-implicated. Also provided is the use of an ADAMTS14 inhibitor in the manufacture of a medicament for use in treating or preventing a disease/condition in which myofibroblasts are pathologically-implicated. Also provided is a method of treating or preventing a disease/condition in which myofibroblasts are pathologically-implicated, comprising administering to a subject a therapeutically- or prophylactically-effective amount of an ADAMTS14 inhibitor.

It will be appreciated that the methods and articles of the present disclosure may be used for the treatment/prevention of any disease/condition that would derive therapeutic or prophylactic benefit from a reduction in the number and/or activity of myofibroblasts. For example, the disease/condition may be a disease/condition in which an increase in the number and/or activity myofibroblasts is implicated in the pathology of the disease/condition. The present disclosure also provides for the treatment/prevention of diseases/conditions that are caused or exacerbated by myofibroblasts.

The disease/condition may be characterised by an increase in the number and/or activity myofibroblasts (e.g. as compared to the number/activity in the absence of the disease/condition). The disease/condition may be a disease/condition in which an increase in the number and/or activity myofibroblasts is positively associated with the onset, development or progression of the disease/condition. The disease/condition may be a disease/condition in which an increase in the number and/or activity myofibroblasts is positively associated with the severity of one or more symptoms of the disease/condition. The disease/condition may be a disease/condition for which an increase in the number and/or activity myofibroblasts, is a risk factor for the onset, development or progression of the disease/condition.

The increase in the number and/or activity myofibroblasts in accordance with the preceding paragraph may be in tissue and/or an organ in which one or more symptoms of the disease/condition manifest. In some embodiments, the increase in the number and/or activity myofibroblasts may be in tissue and/or an organ of the respiratory system, e.g. the lung.

Therapeutic or prophylactic intervention in accordance with the present disclosure may achieve a reduction in the number and/or activity myofibroblasts (i.e. in the treated subject). In some embodiments, the therapeutic/prophylactic intervention may achieve a reduction in the number and/or activity myofibroblasts in tissue and/or an organ in which one or more symptoms of the disease/condition manifest. In some embodiments, the therapeutic/prophylactic intervention may achieve a reduction in the number and/or activity myofibroblasts in tissue and/or an organ of the respiratory system, e.g. the lung.

In some embodiments, the therapeutic or prophylactic intervention in accordance with the present disclosure is for the treatment/prevention of fibrosis. In some embodiments, a disease/condition in which myofibroblasts are pathologically-implicated is fibrosis, or a disease/condition characterised by fibrosis.

Fibrosis can be triggered by pathological conditions, e.g. conditions, infections or disease states that lead to production of pro-fibrotic factors (e.g. as TGFβ1). Fibrosis may be caused by physical injury/stimuli, chemical injury/stimuli or environmental injury/stimuli. Physical injury/stimuli may occur during surgery, e.g. iatrogenic causes. Chemical injury/stimuli may include drug-induced fibrosis, e.g. following chronic administration of drugs such as bleomycin, cyclophosphamide, amiodarone, procainamide, penicillamine, gold and nitrofurantoin (Daba et al., Saudi Med J. (2004) 25(6): 700-706). Environmental injury/stimuli may include exposure to asbestos fibres or silica.

Fibrosis can be of any tissue/organ of the body. In some embodiments, fibrosis is of the lung (e.g. bronchioles, alveoli), airways (e.g. nasal cavity, oral cavity, pharynx, larynx, trachea, bronchi), heart, kidney, liver, skeletal muscle, blood vessels, eye, skin, pancreas, bowel, small intestine, large intestine, colon, joints, brain, or bone marrow. Fibrosis may also occur in multiple tissues/organs at once.

In some embodiments, fibrosis may be of an organ or tissue of the respiratory system, e.g. the lung (e.g. bronchioles, alveoli), or airways (e.g. nasal cavity, oral cavity, pharynx, larynx, trachea, bronchi). In some embodiments, fibrosis may be of an organ or tissue of the cardiovascular system, e.g. the heart or blood vessels. In some embodiments, fibrosis may be of an organ or tissue of the gastrointestinal system, e.g. of the liver, bowel, small intestine, large intestine, colon, or pancreas. In some embodiments, fibrosis may be of the eye. In some embodiments, fibrosis may be of the skin. In some embodiments, fibrosis may be of an organ or tissue of the nervous system, e.g. the brain. In some embodiments, fibrosis may be of the bone marrow. In some embodiments, fibrosis may be of the joints. In some embodiments, fibrosis may be of an organ or tissue of the urogenital system, e.g. the kidneys ovaries, or fallopian tubes. In some embodiments, fibrosis may be of an organ or tissue of the musculoskeletal system, e.g. muscle tissue. In some embodiments, fibrosis may be of an organ or tissue of one or more organ systems.

The present disclosure contemplates therapeutic and prophylactic intervention for diseases and conditions characterised by fibrosis. As used herein, a disease/condition which is ‘characterised by fibrosis’ is a disease in which fibrosis is a symptom of the disease/condition.

Diseases and conditions characterised by fibrosis include, but are not limited to:
Diseases/conditions affecting the respiratory system such as pulmonary fibrosis, interstitial lung disease (ILD), idiopathic interstitial pneumonia (IIP), idiopathic pulmonary fibrosis (IPF), cystic fibrosis, progressive massive fibrosis, scleroderma, obliterative bronchiolitis, Hermansky-Pudlak syndrome, asbestosis, silicosis, sarcoidosis, tumor stroma in lung disease, chronic obstructive pulmonary disease (COPD), emphysema, chronic bronchitis and asthma;
Diseases/conditions affecting the liver such as chronic liver disease, liver fibrosis, cirrhosis, non-alcoholic fatty liver disease (NAFLD), steatohepatitis, non-alcoholic steatohepatitis (NASH), alcoholic liver disease (ALD), alcoholic fatty liver (AFL), alcoholic hepatitis, alcoholic steatohepatitis (ASH), primary biliary cirrhosis (PBC), schistosomal liver disease and hepatocellular carcinoma (HCC);
Diseases/conditions affecting the cardiovascular system such as hypertrophic cardiomyopathy (HCM), dilated cardiomyopathy (DCM), fibrosis of the atrium, atrial fibrillation, fibrosis of the ventricle, ventricular fibrillation, myocardial fibrosis, Brugada syndrome, myocarditis, endomyocardial fibrosis, myocardial infarction, fibrotic vascular disease, hypertension, hypertensive heart disease, arrhythmogenic right ventricular cardiomyopathy (ARVC), atherosclerosis, chronic pulmonary hypertension, AIDS-associated pulmonary hypertension, varicose veins and cerebral infarcts;
Diseases/conditions affecting the kidneys such as tubulointerstitial fibrosis, glomerular fibrosis, renal fibrosis, nephritic syndrome, Alport's syndrome, HIV-associated nephropathy, polycystic kidney disease, Fabry's disease, diabetic nephropathy, chronic glomerulonephritis and nephritis associated with systemic lupus;
Diseases/conditions affecting the pancreas such as pancreatic fibrosis, cystic fibrosis and chronic pancreatitis;
Diseases/conditions affecting the urogenital system such as endometriosis.
Diseases/conditions affecting the nervous system such as gliosis, Alzheimer's disease and multiple sclerosis;
Diseases/conditions affecting the musculoskeletal system such as muscular dystrophy, Duchenne muscular dystrophy (DMD), Becker's muscular dystrophy (BMD) and fibrotic myopathy;
Diseases/conditions affecting the gastrointestinal system such as inflammatory bowel disease (IBD), Crohn's disease, microscopic colitis and primary sclerosing cholangitis (PSC);
Diseases/conditions affecting the skin such as scleroderma, nephrogenic systemic fibrosis, Dupuytren's contracture and cutis keloid;
Diseases/conditions affecting the eye such as Grave's opthalmopathy, epiretinal fibrosis, retinal fibrosis, subretinal fibrosis, subretinal fibrosis associated with macular degeneration (e.g. wet age-related macular degeneration (AMD)), diabetic retinopathy, glaucoma, corneal fibrosis, post-surgical fibrosis (e.g. of the posterior capsule following cataract surgery, or of the bleb following trabeculectomy for glaucoma), conjunctival fibrosis and subconjunctival fibrosis;
Diseases/conditions affecting the joints such as arthrofibrosis, arthritis and adhesive capsulitis;
Diseases/conditions affecting multiple tissues/organ systems, including progressive systemic sclerosis (PSS), chronic graft versus host disease (GVHD); fibrotic pre-neoplastic and fibrotic neoplastic disease, and fibrosis induced by chemical or environmental insult (e.g., cancer chemotherapy, pesticides, radiation/cancer radiotherapy);
Cancers, such as hepatocellular carcinoma, gastric cancer, esophageal cancer, head and neck cancer, colorectal cancer, pancreatic cancer, cervical cancer, and vulvar cancer;
Mediastinal fibrosis, retroperitoneal fibrosis, myelofibrosis and Peyronie's disease.

It will be appreciated that many of the diseases/conditions recited above are interrelated. For example, fibrosis of the ventricle may occur post myocardial infarction, and is associated with DCM, HCM and myocarditis.

In aspects and embodiments of the present disclosure, an ADAMTS14 inhibitor is provided for use in the treatment or prevention of a disease/condition characterised by fibrosis described herein. Also provided is the use of an ADAMTS14 inhibitor in the manufacture of a medicament for use in treating or preventing a disease/condition characterised by fibrosis described herein. Also provided is a method of treating or preventing a disease/condition characterised by fibrosis described herein, comprising administering to a subject a therapeutically- or prophylactically-effective amount of an ADAMTS14 inhibitor.

Therapeutic or prophylactic intervention in accordance with the present disclosure may be effective to reduce the development or progression of a disease/condition, alleviate the symptoms of a disease/condition or reduce the pathology of a disease/condition. The intervention may be effective to prevent progression of the disease/condition, e.g. to prevent worsening of, or to slow the rate of development of, the disease/condition. In some embodiments the intervention may lead to an improvement in the disease/condition, e.g. a reduction in the symptoms of the disease/condition or reduction in some other correlate of the severity/activity of the disease/condition. In some embodiments the intervention may prevent development of the disease/condition to a later stage (e.g. a more severe stage, or a chronic stage). In some embodiments, the intervention may be aimed at slowing, stopping and/or reversing impairment of tissue/organ function associated with the disease/condition.

Therapeutic or prophylactic intervention in accordance with the present disclosure may be effective to reduce the development or progression of fibrosis, alleviate fibrosis, or reduce fibrosis. The intervention may be effective to prevent progression of fibrosis, e.g. to prevent worsening of, or to slow the rate of development of, fibrosis. In some embodiments the intervention may lead to an improvement, e.g. a reduction in fibrosis. In some embodiments the intervention may prevent development of fibrosis to a later stage (e.g. a more severe stage). In some embodiments, the intervention may be aimed at slowing, stopping and/or reversing impairment of tissue/organ function associated with fibrosis.

In aspects and embodiments of the present disclosure, an ADAMTS14 inhibitor is provided for use in the treatment or prevention of fibrosis in a disease/condition characterised by fibrosis described herein. Also provided is the use of an ADAMTS14 inhibitor in the manufacture of a medicament for use in treating or preventing fibrosis in a disease/condition characterised by fibrosis described herein. Also provided is a method of treating or preventing fibrosis in a disease/condition characterised by fibrosis described herein, comprising administering to a subject a therapeutically- or prophylactically-effective amount of an ADAMTS14 inhibitor. That is, in aspects and embodiments of the present disclosure the ADAMTS14 inhibitor is provided for use in the treatment or prevention of the fibrotic component of the relevant disease/condition.

Fibrosis can lead directly or indirectly to, and/or increase susceptibility to development of, certain diseases and conditions. For example, more than 80% of hepatocellular carcinomas (HCCs) develop in fibrotic or cirrhotic livers (Affo et al. 2016, Annu Rev Pathol.), suggesting an important role for liver fibrosis in the premalignant environment (PME) of the liver.

Accordingly, the present disclosure also provides ADAMTS14 inhibitors for use in the treatment and prevention of diseases associated with fibrosis, and/or for which fibrosis is a risk factor. In some embodiments, the disease associated with fibrosis, or for which fibrosis is a risk factor, is a cancer, e.g. cancer of the liver (e.g. hepatocellular carcinoma).

In some embodiments, the disease/condition to be treated in accordance with the present disclosure may be characterised by an increased level of expression (i.e. gene and/or protein expression) of ADAMTS14, and/or an increased level of ADAMTS14-mediated function (e.g. as compared to the number/activity in the absence of the disease/condition). The disease/condition may be a disease/condition in which an increased level of expression of ADAMTS14, and/or an increased level of ADAMTS14-mediated function is positively associated with the onset, development or progression of the disease/condition. The disease/condition may be a disease/condition in which an increased level of expression of ADAMTS14, and/or an increased level of ADAMTS14-mediated function is positively associated with the severity of one or more symptoms of the disease/condition. The disease/condition may be a disease/condition for which an increased level of expression of ADAMTS14, and/or an increased level of ADAMTS14 function, is a risk factor for the onset, development or progression of the disease/condition.

The increase in the level of expression of ADAMTS14 and/or ADAMTS14-mediated function may be in in cells (e.g. fibroblasts) of a tissue and/or an organ in which one or more symptoms of the disease/condition manifest. In some embodiments, the increase in the number and/or activity myofibroblasts may be in cells (e.g. fibroblasts) of a tissue and/or an organ of the respiratory system, e.g. the lung.

Therapeutic or prophylactic intervention in accordance with the present disclosure may achieve a reduction in the level of expression of ADAMTS14 and/or in the level of an ADAMTS14-mediated function (i.e. in the treated subject). In some embodiments, the therapeutic/prophylactic intervention may achieve a reduction in the level of expression of ADAMTS14 and/or in the level of an ADAMTS14-mediated function in cells (e.g. fibroblasts) of a tissue and/or an organ in which one or more symptoms of the disease/condition manifest. In some embodiments, the therapeutic/prophylactic intervention may achieve a reduction in the level of expression of ADAMTS14 and/or in the level of an ADAMTS14-mediated function in cells (e.g. fibroblasts) of a tissue and/or an organ of the respiratory system, e.g. the lung.

In some embodiments, the therapeutic or prophylactic intervention in accordance with the present disclosure is for the treatment/prevention of fibrosis of an organ/tissue of the respiratory system, e.g. selected from the lung, bronchioles, alveoli, airways, nasal cavity, oral cavity, pharynx, larynx, trachea and bronchi.

In some embodiments, the therapeutic or prophylactic intervention in accordance with the present disclosure is for the treatment/prevention of a disease/condition affecting an organ/tissue of the respiratory system, e.g. selected from pulmonary fibrosis, interstitial lung disease (ILD), idiopathic interstitial pneumonia (IIP), idiopathic pulmonary fibrosis (IPF), cystic fibrosis, progressive massive fibrosis, scleroderma, obliterative bronchiolitis, Hermansky-Pudlak syndrome, asbestosis, silicosis, sarcoidosis, tumor stroma in lung disease, chronic obstructive pulmonary disease (COPD), emphysema, chronic bronchitis and asthma. In some embodiments, the therapeutic or prophylactic intervention in accordance with the present disclosure is for the treatment/prevention of fibrosis in such a disease/condition.

In some embodiments, the present disclosure provides an ADAMTS14 inhibitor for the treatment or prevention of fibrosis of the lung (i.e. pulmonary fibrosis). In some embodiments, the present disclosure provides an ADAMTS14 inhibitor for the treatment or prevention of interstitial lung disease (ILD). In some embodiments, the present disclosure provides an ADAMTS14 inhibitor for the treatment or prevention of idiopathic interstitial pneumonia (IIP). In some embodiments, the present disclosure provides an ADAMTS14 inhibitor for the treatment or prevention of idiopathic pulmonary fibrosis (IPF). Idiopathic pulmonary fibrosis (IPF) is reviewed e.g. in Barratt et al., J Clin Med. (2018) August; 7(8): 201, which is hereby incorporated by reference in its entirety.

In accordance with various aspects of the present disclosure, methods are provided which are for, or which comprise (e.g. in the context of therapeutic/prophylactic intervention as described herein), one or more of the following: reducing expression (e.g. gene and/or protein expression) of ADAMTS14; reducing the level of RNA encoding ADAMTS14; reducing transcription of nucleic acid encoding ADAMTS14; increasing degradation of RNA encoding ADAMTS14; reducing the level of ADAMTS14 protein; reducing post-transcriptional processing (e.g. splicing, translation, post-translational processing) of RNA encoding ADAMTS14; increasing degradation of ADAMTS14 protein; reducing the level of a correlate of ADAMTS14 activity; reducing cleavage of type I aminoprocollagen; reducing expression of one or more genes involved in fibrosis and/or metaplastic differentiation (e.g. one or more genes selected from ADAMTS14, ACTA2, COL1A1, FN1, FOXJ1, KRT5, MUC5AC, MUC5B, SCGB1A1, CTGF, CYR61, MMP7 and SOX2; e.g. in fibroblasts or epithelial cells); reducing the level of YAP (e.g. in fibroblasts); reducing nuclear localisation of YAP (e.g. in fibroblasts); increasing degradation of YAP (e.g. in fibroblasts); increasing the level of phosphorylated YAP (e.g. YAP phosphorylated at S397; e.g. in fibroblasts); reducing the level of a protein encoded by a gene whose expression is upregulated by YAP (e.g. in fibroblasts); reducing the level of TAZ (e.g. in fibroblasts); reducing nuclear localisation of TAZ (e.g. in fibroblasts); increasing the level of phosphorylated TAZ (e.g. TAZ phosphorylated at S89; e.g. in fibroblasts); reducing the level of a protein encoded by a gene whose expression is upregulated by TAZ (e.g. in fibroblasts); reducing the level of CTGF (e.g. in fibroblasts); reducing the level of CYR61 (e.g. in fibroblasts); reducing TGFβ1-mediated signalling (e.g. in fibroblasts); reducing the level of phosphorylated SMAD2 (e.g. in fibroblasts); reducing localisation of SMAD2 to the nucleus (e.g. in fibroblasts); reducing the level of αSMA (e.g. in fibroblasts); reducing production of a component of extracellular matrix (e.g. type 1 collagen I; e.g. in/by fibroblasts) reducing extracellular matrix stiffness (e.g. of extracellular matrix produced by fibroblasts or epithelial cells in culture in vitro); and/or reducing the stiffness of a tissue/organ (e.g. lung, liver, skin, kidney), e.g. a tissue/organ comprising fibrosis.

Also provided are agents according to the present disclosure for use in such methods, and the use of agents according to the present disclosure in manufacture of compositions (e.g. medicaments) for use in such methods. It will be appreciated that the methods comprise administering an ADAMTS14 inhibitor to a subject.

Similarly, one or more of the following may be observed in a subject (or a tissue/organ thereof) following therapeutic or prophylactic intervention in accordance with the present disclosure (e.g. compared to the level prior to intervention): reduced expression (e.g. gene and/or protein expression) of ADAMTS14; reduced level of RNA encoding ADAMTS14; reduced transcription of nucleic acid encoding ADAMTS14; increased degradation of RNA encoding ADAMTS14; reduced level of ADAMTS14 protein; reduced post-transcriptional processing (e.g. splicing, translation, post-translational processing) of RNA encoding ADAMTS14; increased degradation of ADAMTS14 protein; reduced level of a correlate of ADAMTS14 activity; reduced cleavage of type I aminoprocollagen; reduced expression of one or more genes involved in fibrosis and/or metaplastic differentiation (e.g. one or more genes selected from ADAMTS14, ACTA2, COL1A1, FN1, FOXJ1, KRT5, MUC5AC, MUC5B, SCGB1A1, CTGF, CYR61, MMP7 and SOX2; e.g. in fibroblasts or epithelial cells); reduced level of YAP (e.g. in fibroblasts); reduced nuclear localisation of YAP (e.g. in fibroblasts); increased degradation of YAP (e.g. in fibroblasts); increased level of phosphorylated YAP (e.g. YAP phosphorylated at S397; e.g. in fibroblasts); reduced level of a protein encoded by a gene whose expression is upregulated by YAP (e.g. in fibroblasts); reduced level of TAZ (e.g. in fibroblasts); reduced nuclear localisation of TAZ (e.g. in fibroblasts); increased level of phosphorylated TAZ (e.g. TAZ phosphorylated at S89; e.g. in fibroblasts); reduced level of a protein encoded by a gene whose expression is upregulated by TAZ (e.g. in fibroblasts); reduced level of CTGF (e.g. in fibroblasts); reduced level of CYR61 (e.g. in fibroblasts); reduced TGFβ1-mediated signalling (e.g. in fibroblasts); reduced level of phosphorylated SMAD2 (e.g. in fibroblasts); reduced localisation of SMAD2 to the nucleus (e.g. in fibroblasts); reduced level of αSMA (e.g. in fibroblasts); reduced production of a component of extracellular matrix (e.g. type 1 collagen I; e.g. in/by fibroblasts); reduced extracellular matrix stiffness (e.g. of extracellular matrix produced by fibroblasts or epithelial cells in culture in vitro); and/or reduced stiffness of a tissue/organ (e.g. lung, liver, skin, kidney), e.g. a tissue/organ comprising fibrosis.

In some embodiments, therapeutic/prophylactic intervention in accordance with the present disclosure may be described as being ‘associated with’ one or more of the effects described in the preceding paragraph. The skilled person is readily able to evaluate such properties using techniques that are routinely practiced in the art.

Administration of the articles of the present disclosure is preferably in a ‘therapeutically-effective’ or ‘prophylactically-effective’ amount, this being sufficient to show therapeutic or prophylactic benefit to the subject. The actual amount administered, and rate and time-course of administration, will depend on the nature and severity of the disease/condition and the particular article administered. Prescription of treatment, e.g. decisions on dosage etc., is within the responsibility of general practitioners and other medical doctors, and typically takes account of the disease/disorder to be treated, the condition of the individual subject, the site of delivery, the method of administration and other factors known to practitioners. Examples of the techniques and protocols mentioned above can be found in Remington's ‘The Science and Practice of Pharmacy’ (ed. A. Adejare), 23rd Edition (2020), Academic Press.

Administration of the articles of the present disclosure may be parenteral, systemic, intracavitary, intravenous, intra-arterial, intramuscular, intrathecal, topical, intraocular, intravitreal, intraconjunctival, subretinal, suprachoroidal, subcutaneous, intradermal, intrathecal, oral, nasal or transdermal. Administration may be by injection, infusion, inhalation or nebulisation.

In some aspects and embodiments, articles of the present disclosure may be administered to the lung. In some cases, articles of the present disclosure may be administered to the blood (i.e. intravenous/intra-arterial administration), subcutaneously or orally.

In some aspects and embodiments in accordance with the present disclosure there may be targeted delivery of articles of the present disclosure, i.e. wherein the concentration of the relevant agent in the subject is increased in a given tissue(s)/organ(s) relative to other parts of the body. In some embodiments, the methods comprise intravenous, intra-arterial, intramuscular or subcutaneous administration, or administration by inhalation of nebulisation and wherein the relevant article is formulated in a targeted agent delivery system. Suitable targeted delivery systems include, for example, nanoparticles, liposomes, micelles, beads, polymers, metal particles, dendrimers, antibodies, aptamers, nanotubes or micro-sized silica rods. Such systems may comprise a magnetic element to direct the agent to the desired organ or tissue. Suitable nanocarriers and delivery systems will be apparent to one skilled in the art.

In some embodiments, administration may be by inhalation of vector encoding an ADAMTS14 inhibitor according to the present disclosure, e.g. an adeno-associated virus (AAV) vector or a lentiviral vector encoding the ADAMTS14 inhibitor.

In some cases, the relevant agent is formulated for targeted delivery to specific cells (e.g. fibroblasts), tissue(s) and/or organ(s) (e.g. of tissues/organs of the respiratory system, e.g. the lung). In some cases, the relevant agent is formulated for targeted delivery to fibroblasts. In some cases, the relevant agent is formulated for targeted delivery to cells (e.g. fibroblasts) of tissue(s)/organ(s) of the respiratory system. In some cases, the relevant agent is formulated for targeted delivery to cells (e.g. fibroblasts) of the lung.

The particular mode and/or site of administration may be selected in accordance with the location where inhibition of ADAMTS14 is required, e.g. cells (e.g. fibroblasts) of the tissue(s)/organ(s) of the respiratory system (e.g. the lung).

In some embodiments, therapeutic or prophylactic intervention according to the present disclosure may further comprise administering another agent for the treatment/prevention of the relevant disease/condition.

Administration of an article of the present disclosure may be alone or in combination with other treatments, either simultaneously or sequentially dependent upon the condition to be treated.

Simultaneous administration refers to administration with another therapeutic agent together, for example as a pharmaceutical composition containing both agents (combined preparation), or immediately after each other and optionally via the same route of administration (e.g. to the same tissue, artery, vein or other blood vessel). Sequential administration refers to administration of one agent followed after a given time interval by separate administration of another agent. It is not required that the two agents are administered by the same route, although this is the case in some embodiments. The time interval may be any time interval.

Multiple doses of the articles of the present disclosure may be provided. One or more, or each, of the doses may be accompanied by simultaneous or sequential administration of another therapeutic agent.

Multiple doses may be separated by a predetermined time interval, which may be selected to be one of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or31 days, or 1, 2, 3, 4, 5, or 6 months. By way of example, doses may be given once every 7, 14, 21 or 28 days (plus or minus 3, 2, or 1 days).

Articles of the present disclosure may be formulated in a sustained release delivery system, in order to release the inhibitory nucleic acid, nucleic acid, expression vector or composition at a predetermined rate. Sustained release delivery systems may maintain a constant drug/therapeutic/prophylactic concentration for a specified period of time. In some embodiments, articles of the present disclosure are formulated in a liposome, gel, implant, device, or drug-polymer conjugate e.g. hydrogel.

### Subjects

A subject in accordance with the various aspects of the present disclosure may be any animal or human. Therapeutic and prophylactic applications may be in humans, or in animals (i.e. in the case veterinary use).

The subject to be administered with an article of the present disclosure may be a subject in need of such intervention. The subject is preferably mammalian, more preferably human. In some embodiments, the subject may be a non-human mammal. The subject may be male or female. The subject may be a patient.

A subject may have been diagnosed with a disease or condition described herein, may be suspected of having such a disease/condition, or may be at risk of developing/contracting such a disease/condition. In embodiments according to the present disclosure, a subject may be selected for treatment according to the methods based on characterisation for certain markers of such disease/condition.

### Sequence Identity

As used herein, ‘sequence identity’ refers to the percent of nucleotides/amino acid residues in a subject sequence that are identical to nucleotides/amino acid residues in a reference sequence, after aligning the sequences and, if necessary, introducing gaps, to achieve the maximum percent sequence identity between the sequences. Pairwise and multiple sequence alignment for the purposes of determining percent sequence identity between two or more amino acid or nucleic acid sequences can be achieved in various ways known to a person of skill in the art, for instance, using publicly available computer software such as ClustalOmega (Söding, J. 2005, Bioinformatics 21, 951-960), T-coffee (Notredame et al. 2000, J. Mol. Biol. (2000) 302, 205-217), Kalign (Lassmann and Sonnhammer 2005, BMC Bioinformatics, 6(298)) and MAFFT (Katoh and Standley 2013, Molecular Biology and Evolution, 30(4) 772-780 software. When using such software, the default parameters, e.g. for gap penalty and extension penalty, are preferably used.

### Sequences

| SEQ ID | | |
|---|---|---|
| NO: | DESCRIPTION | SEQUENCE |
| 1 | Human ADAMTS14 | MAPLRALLSYLLPLHCALCAAAGSRTPELHLSGKLSDYGVTVPCSTDFRGR |
| | isoform A | FLSHVVSGPAAASAGSMVVDTPPTLPRHSSHLRVARSPLHPGGTLWPGRV |
| | (UniProtKB: | GRHSLYFNVTVFGKELHLRLRPNRRLVVPGSSVEWQEDFRELFRQPLRQE |
| | Q8WXS8-1, v2) | CVYTGGVTGMPGAAVAISNCDGLAGLIRTDSTDFFIEPLERGQQEKEASGR |
| | | THVVYRREAVQQEWAEPDGDLHNEAFGLGDLPNLLGLVGDQLGDTERKR |
| | | RHAKPGSYSIEVLLVVDDSVVRFHGKEHVQNYVLTLMNIVDEIYHDESLGVH |
| | | INIALVRLIMVGYRQSLSLIERGNPSRSLEQVCRWAHSQQRQDPSHAEHHD |
| | | HVVFLTRQDFGPSGYAPVTGMCHPLRSCALNHEDGFSSAFVIAHETGHVL |
| | | GMEHDGQGNGCADETSLGSVMAPLVQAAFHRFHWSRCSKLELSRYLPSY |
| | | DCLLDDPFDPAWPQPPELPGINYSMDEQCRFDFGSGYQTCLAFRTFEPCK |
| | | QLWCSHPDNPYFCKTKKGPPLDGTECAPGKWCFKGHCIWKSPEQTYGQD |
| | | GGWSSWTKFGSCSRSCGGGVRSRSRSCNNPSPAYGGRLCLGPMFEYQV |
| | | CNSEECPGTYEDFRAQQCAKRNSYYVHQNAKHSWVPYEPDDDAQKCELI |
| | | CQSADTGDVVFMNQVVHDGTRCSYRDPYSVCARGECVPVGCDKEVGSM |
| | | KADDKCGVCGGDNSHCRTVKGTLGKASKQAGALKLVQIPAGARHIQIEALE |
| | | KSPHRIVVKNQVTGSFILNPKGKEATSRTFTAMGLEWEDAVEDAKESLKTS |
| | | GPLPEAIAILALPPTEGGPRSSLAYKYVIHEDLLPLIGSNNVLLEEMDTYEWA |
| | | LKSWAPCSKACGGGIQFTKYGCRRRRDHHMVQRHLCDHKKRPKPIRRRC |
| | | NQHPCSQPVWVTEEWGACSRSCGKLGVQTRGIQCLLPLSNGTHKVMPAK |
| | | ACAGDRPEARRPCLRVPCPAQWRLGAWSQCSATCGEGIQQRQVVCRTN |
| | | ANSLGHCEGDRPDTVQVCSLPACGGNHQNSTVRADVWELGTPEGQWVP |
| | | QSEPLHPINKISSTEPCTGDRSVFCQMEVLDRYCSIPGYHRLCCVSCIKKAS |
| | | GPNPGPDPGPTSLPPFSTPGSPLPGPQDPADAAEPPGKPTGSEDHQHGR |
| | | ATQLPGALDTSSPGTQHPFAPETPIPGASWSISPTTPGGLPWGWTQTPTP |
| | | VPEDKGQPGEDLRHPGTSLPAASPVT |
| 2 | Human ADAMTS14 | MVVDTPPTLPRHSSHLRVARSPLHPGGTLWPGRVGRHSLYFNVTVFGKEL |
| | isoform B | HLRLRPNRRLVVPGSSVEWQEDFRELFRQPLRQECVYTGGVTGMPGAAV |
| | (UniProtKB: | AISNCDGLAGLIRTDSTDFFIEPLERGQQEKEASGRTHVVYRREAVQQEWA |
| | Q8WXS8-2) | EPDGDLHNEAFGLGDLPNLLGLVGDQLGDTERKRRHAKPGSYSIEVLLVVD |
| | | DSVVRFHGKEHVQNYVLTLMNIVDEIYHDESLGVHINIALVRLIMVGYRQSLS |
| | | LIERGNPSRSLEQVCRWAHSQQRQDPSHAEHHDHVVFLTRQDFGPSGYA |
| | | PVTGMCHPLRSCALNHEDGFSSAFVIAHETGHVLGMEHDGQGNGCADET |
| | | SLGSVMAPLVQAAFHRFHWSRCSKLELSRYLPSYDCLLDDPFDPAWPQPP |
| | | ELPGINYSMDEQCRFDFGSGYQTCLAFRTFEPCKQLWCSHPDNPYFCKTK |
| | | KGPPLDGTECAPGKWCFKGHCIWKSPEQTYGQDGGWSSWTKFGSCSRS |
| | | CGGGVRSRSRSCNNPSPAYGGRLCLGPMFEYQVCNSEECPGTYEDFRAQ |
| | | QCAKRNSYYVHQNAKHSWVPYEPDDDAQKCELICQSADTGDVVFMNQVV |
| | | HDGTRCSYRDPYSVCARGECVPVGCDKEVGSMKADDKCGVCGGDNSHC |
| | | RTVKGTLGKASKQAGALKLVQIPAGARHIQIEALEKSPHRIVVKNQVTGSFIL |
| | | NPKGKEATSRTFTAMGLEWEDAVEDAKESLKTSGPLPEAIAILALPPTEGGP |
| | | RSSLAYKYVIHEDLLPLIGSNNVLLEEMDTYEWALKSWAPCSKACGGGIQF |
| | | TKYGCRRRRDHHMVQRHLCDHKKRPKPIRRRCNQHPCSQPVWVTEEWG |
| | | ACSRSCGKLGVQTRGIQCLLPLSNGTHKVMPAKACAGDRPEARRPCLRVP |
| | | CPAQWRLGAWSQCSATCGEGIQQRQVVCRTNANSLGHCEGDRPDTVQV |
| | | CSLPACGGNHQNSTVRADVWELGTPEGQWVPQSEPLHPINKISSTEPCTG |
| | | DRSVFCQMEVLDRYCSIPGYHRLCCVSCIKKASGPNPGPDPGPTSLPPFST |
| | | PGSPLPGPQDPADAAEPPGKPTGSEDHQHGRATQLPGALDTSSPGTQHP |
| | | FAPETPIPGASWSISPTTPGGLPWGWTQTPTPVPEDKGQPGEDLRHPGTS |
| | | LPAASPVT |
| 3 | Human ADAMTS14 | MVVDTPPTLPRHSSHLRVARSPLHPGGTLWPGRVGRHSLYFNVTVFGKEL |
| | isoform C | HLRLRPNRRLVVPGSSVEWQEDFRELFRQPLRQECVYTGGVTGMPGAAV |
| | (UniProtKB: | AISNCDGLAGLIRTDSTDFFIEPLERGQQEKEASGRTHVVYRREAVQQEWA |
| | Q8WXS8-4) | EPDGDLHNEAFGLGDLPNLLGLVGDQLGDTERKRRHAKPGSYSIEVLLVVD |
| | | DSVVRFHGKEHVQNYVLTLMNIVDEIYHDESLGVHINIALVRLIMVGYRQSLS |
| | | LIERGNPSRSLEQVCRWAHSQQRQDPSHAEHHDHVVFLTRQDFGPSGMQ |
| | | GYAPVTGMCHPLRSCALNHEDGFSSAFVIAHETGHVLGMEHDGQGNGCA |
| | | DETSLGSVMAPLVQAAFHRFHWSRCSKLELSRYLPSYDCLLDDPFDPAWP |
| | | QPPELPGINYSMDEQCRFDFGSGYQTCLAFRTFEPCKQLWCSHPDNPYFC |
| | | KTKKGPPLDGTECAPGKWCFKGHCIWKSPEQTYGQDGGWSSWTKFGSC |
| | | SRSCGGGVRSRSRSCNNPSPAYGGRLCLGPMFEYQVCNSEECPGTYEDF |
| | | RAQQCAKRNSYYVHQNAKHSWVPYEPDDDAQKCELICQSADTGDVVFMN |
| | | QVVHDGTRCSYRDPYSVCARGECVPVGCDKEVGSMKADDKCGVCGGDN |
| | | SHCRTVKGTLGKASKQAGALKLVQIPAGARHIQIEALEKSPHRIVVKNQVTG |
| | | SFILNPKGKEATSRTFTAMGLEWEDAVEDAKESLKTSGPLPEAIAILALPPTE |
| | | GGPRSSLAYKYVIHEDLLPLIGSNNVLLEEMDTYEWALKSWAPCSKACGGG |
| | | IQFTKYGCRRRRDHHMVQRHLCDHKKRPKPIRRRCNQHPCSQPVWVTEE |
| | | WGACSRSCGKLGVQTRGIQCLLPLSNGTHKVMPAKACAGDRPEARRPCL |
| | | RVPCPAQWRLGAWSQCSATCGEGIQQRQVVCRTNANSLGHCEGDRPDT |
| | | VQVCSLPACGGNHQNSTVRADVWELGTPEGQWVPQSEPLHPINKISSTEP |
| | | CTGDRSVFCQMEVLDRYCSIPGYHRLCCVSCIKKASGPNPGPDPGPTSLP |
| | | PFSTPGSPLPGPQDPADAAEPPGKPTGSEDHQHGRATQLPGALDTSSPGT |
| | | QHPFAPETPIPGASWSISPTTPGGLPWGWTQTPTPVPEDKGQPGEDLRHP |
| | | GTSLPAASPVT |
| 4 | Human ADAMTS14 | MAPLRALLSYLLPLHCALCAAAGSRTPELHLSGKLSDYGVTVPCSTDFRGR |
| | isoform D | FLSHVVSGPAAASAGSMVVDTPPTLPRHSSHLRVARSPLHPGGTLWPGRV |
| | (UniProtKB: | GRHSLYFNVTVFGKELHLRLRPNRRLVVPGSSVEWQEDFRELFRQPLRQE |
| | Q8WXS8-4) | CVYTGGVTGMPGAAVAISNCDGLAGLIRTDSTDFFIEPLERGQQEKEASGR |
| | | THVVYRREAVQQEWAEPDGDLHNEAFGLGDLPNLLGLVGDQLGDTERKR |
| | | RHAKPGSYSIEVLLVVDDSVVRFHGKEHVQNYVLTLMNIVDEIYHDESLGVH |
| | | INIALVRLIMVGYRQSLSLIERGNPSRSLEQVCRWAHSQQRQDPSHAEHHD |
| | | HVVFLTRQDFGPSGMQGYAPVTGMCHPLRSCALNHEDGFSSAFVIAHETG |
| | | HVLGMEHDGQGNGCADETSLGSVMAPLVQAAFHRFHWSRCSKLELSRYL |
| | | PSYDCLLDDPFDPAWPQPPELPGINYSMDEQCRFDFGSGYQTCLAFRTFE |
| | | PCKQLWCSHPDNPYFCKTKKGPPLDGTECAPGKWCFKGHCIWKSPEQTY |
| | | GQDGGWSSWTKFGSCSRSCGGGVRSRSRSCNNPSPAYGGRLCLGPMFE |
| | | YQVCNSEECPGTYEDFRAQQCAKRNSYYVHQNAKHSWVPYEPDDDAQK |
| | | CELICQSADTGDVVFMNQVVHDGTRCSYRDPYSVCARGECVPVGCDKEV |
| | | GSMKADDKCGVCGGDNSHCRTVKGTLGKASKQAGALKLVQIPAGARHIQI |
| | | EALEKSPHRIVVKNQVTGSFILNPKGKEATSRTFTAMGLEWEDAVEDAKES |
| | | LKTSGPLPEAIAILALPPTEGGPRSSLAYKYVIHEDLLPLIGSNNVLLEEMDTY |
| | | EWALKSWAPCSKACGGGIQFTKYGCRRRRDHHMVQRHLCDHKKRPKPIR |
| | | RRCNQHPCSQPVWVTEEWGACSRSCGKLGVQTRGIQCLLPLSNGTHKVM |
| | | PAKACAGDRPEARRPCLRVPCPAQWRLGAWSQCSATCGEGIQQRQVVC |
| | | RTNANSLGHCEGDRPDTVQVCSLPACGGNHQNSTVRADVWELGTPEGQ |
| | | WVPQSEPLHPINKISSTEPCTGDRSVFCQMEVLDRYCSIPGYHRLCCVSCI |
| | | KKASGPNPGPDPGPTSLPPFSTPGSPLPGPQDPADAAEPPGKPTGSEDHQ |
| | | HGRATQLPGALDTSSPGTQHPFAPETPIPGASWSISPTTPGGLPWGWTQT |
| | | PTPVPEDKGQPGEDLRHPGTSLPAASPVT |
| 5 | Human ADAMTS14 | MAPLRALLSYLLPLHCALCAAA |
| | isoforms A, D | |
| | signal peptide | |
| 6 | Human ADAMTS14 | GSRTPELHLSGKLSDYGVTVPCSTDFRGRFLSHVVSGPAAASAGSMVVDT |
| | isoforms A, D | PPTLPRHSSHLRVARSPLHPGGTLWPGRVGRHSLYFNVTVFGKELHLRLR |
| | propeptide | PNRRLVVPGSSVEWQEDFRELFRQPLRQECVYTGGVTGMPGAAVAISNC |
| | | DGLAGLIRTDSTDFFIEPLERGQQEKEASGRTHVVYRREAVQQEWAEPDG |
| | | DLHNEAFGLGDLPNLLGLVGDQLGDTERKRR |
| 7 | Human ADAMTS14 | MVVDTPPTLPRHSSHLRVARSPLHPGGTLWPGRVGRHSLYFNVTVFGKEL |
| | isoforms B, C | HLRLRPNRRLVVPGSSVEWQEDFRELFRQPLRQECVYTGGVTGMPGAAV |
| | propeptide | AISNCDGLAGLIRTDSTDFFIEPLERGQQEKEASGRTHVVYRREAVQQEWA |
| | | EPDGDLHNEAFGLGDLPNLLGLVGDQLGDTERKRR |
| 8 | Human ADAMTS14 | HAKPGSYSIEVLLVVDDSVVRFHGKEHVQNYVLTLMNIVDEIYHDESLGVHI |
| | isoforms A, B | NIALVRLIMVGYRQSLSLIERGNPSRSLEQVCRWAHSQQRQDPSHAEHHD |
| | mature protein | HVVFLTRQDFGPSGYAPVTGMCHPLRSCALNHEDGFSSAFVIAHETGHVL |
| | | GMEHDGQGNGCADETSLGSVMAPLVQAAFHRFHWSRCSKLELSRYLPSY |
| | | DCLLDDPFDPAWPQPPELPGINYSMDEQCRFDFGSGYQTCLAFRTFEPCK |
| | | QLWCSHPDNPYFCKTKKGPPLDGTECAPGKWCFKGHCIWKSPEQTYGQD |
| | | GGWSSWTKFGSCSRSCGGGVRSRSRSCNNPSPAYGGRLCLGPMFEYQV |
| | | CNSEECPGTYEDFRAQQCAKRNSYYVHQNAKHSWVPYEPDDDAQKCELI |
| | | CQSADTGDVVFMNQVVHDGTRCSYRDPYSVCARGECVPVGCDKEVGSM |
| | | KADDKCGVCGGDNSHCRTVKGTLGKASKQAGALKLVQIPAGARHIQIEALE |
| | | KSPHRIVVKNQVTGSFILNPKGKEATSRTFTAMGLEWEDAVEDAKESLKTS |
| | | GPLPEAIAILALPPTEGGPRSSLAYKYVIHEDLLPLIGSNNVLLEEMDTYEWA |
| | | LKSWAPCSKACGGGIQFTKYGCRRRRDHHMVQRHLCDHKKRPKPIRRRC |
| | | NQHPCSQPVWVTEEWGACSRSCGKLGVQTRGIQCLLPLSNGTHKVMPAK |
| | | ACAGDRPEARRPCLRVPCPAQWRLGAWSQCSATCGEGIQQRQVVCRTN |
| | | ANSLGHCEGDRPDTVQVCSLPACGGNHQNSTVRADVWELGTPEGQWVP |
| | | QSEPLHPINKISSTEPCTGDRSVFCQMEVLDRYCSIPGYHRLCCVSCIKKAS |
| | | GPNPGPDPGPTSLPPFSTPGSPLPGPQDPADAAEPPGKPTGSEDHQHGR |
| | | ATQLPGALDTSSPGTQHPFAPETPIPGASWSISPTTPGGLPWGWTQTPTP |
| | | VPEDKGQPGEDLRHPGTSLPAASPVT |
| 9 | Human ADAMTS14 | HAKPGSYSIEVLLVVDDSVVRFHGKEHVQNYVLTLMNIVDEIYHDESLGVHI |
| | isoforms C, D | NIALVRLIMVGYRQSLSLIERGNPSRSLEQVCRWAHSQQRQDPSHAEHHD |
| | mature protein | HVVFLTRQDFGPSGMQGYAPVTGMCHPLRSCALNHEDGFSSAFVIAHETG |
| | | HVLGMEHDGQGNGCADETSLGSVMAPLVQAAFHRFHWSRCSKLELSRYL |
| | | PSYDCLLDDPFDPAWPQPPELPGINYSMDEQCRFDFGSGYQTCLAFRTFE |
| | | PCKQLWCSHPDNPYFCKTKKGPPLDGTECAPGKWCFKGHCIWKSPEQTY |
| | | GQDGGWSSWTKFGSCSRSCGGGVRSRSRSCNNPSPAYGGRLCLGPMFE |
| | | YQVCNSEECPGTYEDFRAQQCAKRNSYYVHQNAKHSWVPYEPDDDAQK |
| | | CELICQSADTGDVVFMNQVVHDGTRCSYRDPYSVCARGECVPVGCDKEV |
| | | GSMKADDKCGVCGGDNSHCRTVKGTLGKASKQAGALKLVQIPAGARHIQI |
| | | EALEKSPHRIVVKNQVTGSFILNPKGKEATSRTFTAMGLEWEDAVEDAKES |
| | | LKTSGPLPEAIAILALPPTEGGPRSSLAYKYVIHEDLLPLIGSNNVLLEEMDTY |
| | | EWALKSWAPCSKACGGGIQFTKYGCRRRRDHHMVQRHLCDHKKRPKPIR |
| | | RRCNQHPCSQPVWVTEEWGACSRSCGKLGVQTRGIQCLLPLSNGTHKVM |
| | | PAKACAGDRPEARRPCLRVPCPAQWRLGAWSQCSATCGEGIQQRQVVC |
| | | RTNANSLGHCEGDRPDTVQVCSLPACGGNHQNSTVRADVWELGTPEGQ |
| | | WVPQSEPLHPINKISSTEPCTGDRSVFCQMEVLDRYCSIPGYHRLCCVSCI |
| | | KKASGPNPGPDPGPTSLPPFSTPGSPLPGPQDPADAAEPPGKPTGSEDHQ |
| | | HGRATQLPGALDTSSPGTQHPFAPETPIPGASWSISPTTPGGLPWGWTQT |
| | | PTPVPEDKGQPGEDLRHPGTSLPAASPVT |
| 10 | Human ADAMTS14 | YSIEVLLVVDDSVVRFHGKEHVQNYVLTLMNIVDEIYHDESLGVHINIALVRLI |
| | isoforms A, B | MVGYRQSLSLIERGNPSRSLEQVCRWAHSQQRQDPSHAEHHDHVVFLTR |
| | peptidase M12B | QDFGPSGYAPVTGMCHPLRSCALNHEDGFSSAFVIAHETGHVLGMEHDG |
| | domain | QGNGCADETSLGSVMAPLVQAAFHRFHWSRCSKLELSRYLPSYDCLLDDP |
| 11 | Human ADAMTS14 | YSIEVLLVVDDSVVRFHGKEHVQNYVLTLMNIVDEIYHDESLGVHINIALVRLI |
| | isoforms C, D | MVGYRQSLSLIERGNPSRSLEQVCRWAHSQQRQDPSHAEHHDHVVFLTR |
| | peptidase M12B | QDFGPSGMQGYAPVTGMCHPLRSCALNHEDGFSSAFVIAHETGHVLGME |
| | domain | HDGQGNGCADETSLGSVMAPLVQAAFHRFHWSRCSKLELSRYLPSYDCLL |
| | | DDP |
| 12 | Human ADAMTS14 | FDPAWPQPPELPGINYSMDEQCRFDFGSGYQTCLAFRTFEPCKQLWCSH |
| | disintegrin | PDNPYFCKTKKGPPLDGTECAPGKWCFKGHCIWKSPEQTYGQ |
| | domain | |
| 13 | Human ADAMTS14 | DGGWSSWTKFGSCSRSCGGGVRSRSRSCNNPSPAYGGRLCLGPMFEYQ |
| | TSP type 1 | VCNSEECP |
| | repeat 1 | |
| 14 | Human ADAMTS14 | DTYEWALKSWAPCSKACGGGIQFTKYGCRRRRDHHMVQRHLCDHKKRPK |
| | TSP type 1 | PIRRRCNQHPCS |
| | repeat 2 | |
| 15 | Human ADAMTS14 | QPVWVTEEWGACSRSCGKLGVQTRGIQCLLPLSNGTHKVMPAKACAGDR |
| | TSP type 1 | PEARRPCLRVP |
| | repeat 3 | |
| 16 | Human ADAMTS14 | CPAQWRLGAWSQCSATCGEGIQQRQVVCRTNANSLGHCEGDRPDTVQV |
| | TSP type 1 | CSLPACG |
| | repeat 4 | |
| 17 | Human ADAMTS14 | LKLVQIPAGARHIQIEALEKSPHRIVVKNQVTGSFILNPKGKEATSRTFTAMG |
| | spacer domain | LEWEDAVEDAKESLKTSGPLPEAIAILALPPTEGGPRSSLAYKYVIHEDLLPLI |
| | | GSNNVLLEEM |
| 18 | Human ADAMTS14 | STEPCTGDRSVFCQMEVLDRYCSIPGYHRLCCVSCIKKA |
| | PLAC domain | |
| 19 | Human ADAMTS14 | PNPGPDPGPTSLPPFSTPGSPLPGPQDPADAAEPPGKPTGSEDHQHGRA |
| | proline-rich | TQLPGALDTSSPGTQHPFAPETPIPGASWSISPTTPGGLPWGWTQTPTPV |
| | region | PEDKGQPGEDLRHPGTSLPAASPVT |

### Numbered Paragraphs

The following numbered paragraphs (paras) provide further statements of features and combinations of features which are contemplated in connection with the present invention:

1. An ADAMTS14 inhibitor for use in the treatment or prevention of fibrosis.

2. Use of an ADAMTS14 inhibitor in the manufacture of a medicament for treating or preventing fibrosis.

3. A method of treating or preventing fibrosis in a subject, comprising administering to a subject a therapeutically- or prophylactically-effective amount of an ADAMTS14 inhibitor.

4. The ADAMTS14 inhibitor for use according to para 1, the use according to para 2, or the method according to para 3, wherein the fibrosis is fibrosis of: an organ or tissue of the respiratory system, lung, bronchioles, alveoli, airways, nasal cavity, oral cavity, pharynx, larynx, trachea, bronchus, an organ or tissue of the cardiovascular system, heart, blood vessels, an organ or tissue of the gastrointestinal system, liver, bowel, small intestine, large intestine, colon, pancreas, skin, eye, an organ or tissue of the nervous system, brain, an organ or tissue of the urogenital system, kidney, ovaries, fallopian tubes, an organ or tissue of the musculoskeletal system, muscle tissue, or bone marrow.

5. The ADAMTS14 inhibitor for use according to para 1 or para 4, the use according to para 2 or para 4, or the method according to para 3 or para 4, wherein the fibrosis is fibrosis of a disease or condition selected from: pulmonary fibrosis, interstitial lung disease (ILD), idiopathic interstitial pneumonia (IIP), idiopathic pulmonary fibrosis (IPF), cystic fibrosis, progressive massive fibrosis, scleroderma, obliterative bronchiolitis, Hermansky-Pudlak syndrome, asbestosis, silicosis, sarcoidosis, tumor stroma in lung disease, chronic obstructive pulmonary disease (COPD), emphysema, chronic bronchitis, asthma, chronic liver disease, liver fibrosis, cirrhosis, non-alcoholic fatty liver disease (NAFLD), steatohepatitis, non-alcoholic steatohepatitis (NASH), alcoholic liver disease (ALD), alcoholic fatty liver (AFL), alcoholic hepatitis, alcoholic steatohepatitis (ASH), primary biliary cirrhosis (PBC), schistosomal liver disease, hepatocellular carcinoma (HCC), hypertrophic cardiomyopathy (HCM), dilated cardiomyopathy (DCM), fibrosis of the atrium, atrial fibrillation, fibrosis of the ventricle, ventricular fibrillation, myocardial fibrosis, Brugada syndrome, myocarditis, endomyocardial fibrosis, myocardial infarction, fibrotic vascular disease, hypertension, hypertensive heart disease, arrhythmogenic right ventricular cardiomyopathy (ARVC), atherosclerosis, chronic pulmonary hypertension, AIDS-associated pulmonary hypertension, varicose veins, cerebral infarcts, tubulointerstitial fibrosis, glomerular fibrosis, renal fibrosis, nephritic syndrome, Alport's syndrome, HIV-associated nephropathy, polycystic kidney disease, Fabry's disease, diabetic nephropathy, chronic glomerulonephritis, nephritis associated with systemic lupus, pancreatic fibrosis, chronic pancreatitis, endometriosis, gliosis, Alzheimer's disease, multiple sclerosis, muscular dystrophy, Duchenne muscular dystrophy (DMD), Becker's muscular dystrophy (BMD), fibrotic myopathy, inflammatory bowel disease (IBD), Crohn's disease, microscopic colitis, primary sclerosing cholangitis (PSC), scleroderma, nephrogenic systemic fibrosis, Dupuytren's contracture, cutis keloid, Grave's opthalmopathy, epiretinal fibrosis, retinal fibrosis, subretinal fibrosis, subretinal fibrosis associated with macular degeneration, wet age-related macular degeneration (AMD), diabetic retinopathy, glaucoma, corneal fibrosis, post-surgical fibrosis, post-surgical fibrosis of the posterior capsule following cataract surgery, post-surgical fibrosis of the bleb following trabeculectomy for glaucoma, conjunctival fibrosis, subconjunctival fibrosis, arthrofibrosis, arthritis, adhesive capsulitis, progressive systemic sclerosis (PSS), chronic graft versus host disease (GVHD), fibrotic pre-neoplastic disease, fibrotic neoplastic disease, fibrosis induced by chemical insult, or fibrosis induced by environmental insult, fibrosis induced by cancer chemotherapy, fibrosis induced by pesticides, fibrosis induced by radiation, fibrosis induced by cancer radiotherapy, cancer, hepatocellular carcinoma, gastric cancer, esophageal cancer, head and neck cancer, colorectal cancer, pancreatic cancer, cervical cancer, vulvar cancer, mediastinal fibrosis, retroperitoneal fibrosis, myelofibrosis and Peyronie's disease.

6. The ADAMTS14 inhibitor for use according to any one of paras 1, 4 or 5, the use according to any one of paras 2, 4 or 5, or the method according to any one of paras para 3 to 5, wherein the fibrosis is fibrosis of: an organ or tissue of the respiratory system, lung, bronchioles, alveoli, airways, nasal cavity, oral cavity, pharynx, larynx, trachea or bronchus.

7. The ADAMTS14 inhibitor for use according to any one of paras 1, or 4 to 6, the use according to any one of paras 2, or 4 to 6, or the method according to any one of paras para 3 to 6, wherein the fibrosis is fibrosis of a disease or condition selected from: pulmonary fibrosis, interstitial lung disease (ILD), idiopathic interstitial pneumonia (IIP), idiopathic pulmonary fibrosis (IPF), cystic fibrosis, progressive massive fibrosis, scleroderma, obliterative bronchiolitis, Hermansky-Pudlak syndrome, asbestosis, silicosis, sarcoidosis, tumor stroma in lung disease, chronic obstructive pulmonary disease (COPD), emphysema, chronic bronchitis and asthma.

8. An ADAMTS14 inhibitor for use in the treatment or prevention of a disease or condition characterised by fibrosis.

9. Use of an ADAMTS14 inhibitor in the manufacture of a medicament for treating or preventing a disease or condition characterised by fibrosis.

10. A method of treating or preventing a disease or condition characterised by fibrosis in a subject, comprising administering to a subject a therapeutically- or prophylactically-effective amount of an ADAMTS14 inhibitor.

11. The ADAMTS14 inhibitor for use according to para 8, the use according to para 9, or the method according to para 10, wherein the disease or condition characterised by fibrosis comprises fibrosis of: an organ or tissue of the respiratory system, lung, bronchioles, alveoli, airways, nasal cavity, oral cavity, pharynx, larynx, trachea, bronchus, an organ or tissue of the cardiovascular system, heart, blood vessels, an organ or tissue of the gastrointestinal system, liver, bowel, small intestine, large intestine, colon, pancreas, skin, eye, an organ or tissue of the nervous system, brain, an organ or tissue of the urogenital system, kidney, ovaries, fallopian tubes, an organ or tissue of the musculoskeletal system, muscle tissue, or bone marrow.

12. The ADAMTS14 inhibitor for use according to para 8 or para 11, the use according to para 9 or para 11, or the method according to para 10 or para 11, wherein the disease or condition characterised by fibrosis is selected from: pulmonary fibrosis, interstitial lung disease (ILD), idiopathic interstitial pneumonia (IIP), idiopathic pulmonary fibrosis (IPF), cystic fibrosis, progressive massive fibrosis, scleroderma, obliterative bronchiolitis, Hermansky-Pudlak syndrome, asbestosis, silicosis, sarcoidosis, tumor stroma in lung disease, chronic obstructive pulmonary disease (COPD), emphysema, chronic bronchitis, asthma, chronic liver disease, liver fibrosis, cirrhosis, non-alcoholic fatty liver disease (NAFLD), steatohepatitis, non-alcoholic steatohepatitis (NASH), alcoholic liver disease (ALD), alcoholic fatty liver (AFL), alcoholic hepatitis, alcoholic steatohepatitis (ASH), primary biliary cirrhosis (PBC), schistosomal liver disease, hepatocellular carcinoma (HCC), hypertrophic cardiomyopathy (HCM), dilated cardiomyopathy (DCM), fibrosis of the atrium, atrial fibrillation, fibrosis of the ventricle, ventricular fibrillation, myocardial fibrosis, Brugada syndrome, myocarditis, endomyocardial fibrosis, myocardial infarction, fibrotic vascular disease, hypertension, hypertensive heart disease, arrhythmogenic right ventricular cardiomyopathy (ARVC), atherosclerosis, chronic pulmonary hypertension, AIDS-associated pulmonary hypertension, varicose veins, cerebral infarcts, tubulointerstitial fibrosis, glomerular fibrosis, renal fibrosis, nephritic syndrome, Alport's syndrome, HIV-associated nephropathy, polycystic kidney disease, Fabry's disease, diabetic nephropathy, chronic glomerulonephritis, nephritis associated with systemic lupus, pancreatic fibrosis, chronic pancreatitis, endometriosis, gliosis, Alzheimer's disease, multiple sclerosis, muscular dystrophy, Duchenne muscular dystrophy (DMD), Becker's muscular dystrophy (BMD), fibrotic myopathy, inflammatory bowel disease (IBD), Crohn's disease, microscopic colitis, primary sclerosing cholangitis (PSC), scleroderma, nephrogenic systemic fibrosis, Dupuytren's contracture, cutis keloid, Grave's opthalmopathy, epiretinal fibrosis, retinal fibrosis, subretinal fibrosis, subretinal fibrosis associated with macular degeneration, wet age-related macular degeneration (AMD), diabetic retinopathy, glaucoma, corneal fibrosis, post-surgical fibrosis, post-surgical fibrosis of the posterior capsule following cataract surgery, post-surgical fibrosis of the bleb following trabeculectomy for glaucoma, conjunctival fibrosis, subconjunctival fibrosis, arthrofibrosis, arthritis, adhesive capsulitis, progressive systemic sclerosis (PSS), chronic graft versus host disease (GVHD), fibrotic pre-neoplastic disease, fibrotic neoplastic disease, fibrosis induced by chemical insult, or fibrosis induced by environmental insult, fibrosis induced by cancer chemotherapy, fibrosis induced by pesticides, fibrosis induced by radiation, fibrosis induced by cancer radiotherapy, cancer, hepatocellular carcinoma, gastric cancer, esophageal cancer, head and neck cancer, colorectal cancer, pancreatic cancer, cervical cancer, vulvar cancer, mediastinal fibrosis, retroperitoneal fibrosis, myelofibrosis and Peyronie's disease.

13. The ADAMTS14 inhibitor for use according to any one of paras 8, 11 or 12, the use according to any one of paras 9, 11 or 12, or the method according to any one of paras 10 to 12, wherein the disease or condition characterised by fibrosis comprises fibrosis of: an organ or tissue of the respiratory system, lung, bronchioles, alveoli, airways, nasal cavity, oral cavity, pharynx, larynx, trachea or bronchus.

14. The ADAMTS14 inhibitor for use according to any one of paras 8, or 11 to 13, the use according to any one of paras 9, or 11 to 13, or the method according to any one of paras 10 to 13, wherein the disease or condition characterised by fibrosis is selected from: pulmonary fibrosis, interstitial lung disease (ILD), idiopathic interstitial pneumonia (IIP), idiopathic pulmonary fibrosis (IPF), cystic fibrosis, progressive massive fibrosis, scleroderma, obliterative bronchiolitis, Hermansky-Pudlak syndrome, asbestosis, silicosis, sarcoidosis, tumor stroma in lung disease, chronic obstructive pulmonary disease (COPD), emphysema, chronic bronchitis and asthma.

15. A method of inhibiting TGFβ1-mediated signalling in a cell, comprising contacting a cell with an ADAMTS14 inhibitor.

16. A method of inhibiting YAP-mediated signalling in a cell, comprising contacting a cell with an ADAMTS14 inhibitor.

17. A method of inhibiting the generation of a pro-fibrotic fibroblast, comprising contacting a pro-fibrotic fibroblast precursor cell with an ADAMTS14 inhibitor.

18. A method of inhibiting a process mediated by pro-fibrotic fibroblasts, comprising contacting a pro-fibrotic fibroblast or a pro-fibrotic fibroblast precursor cell with an ADAMTS14 inhibitor.

19. The ADAMTS14 inhibitor for use according to any one of paras 1, 4 to 7, 8, or 11 to 14, the use according to any one of paras 2, 4 to 7, 9, or 11 to 14, or the method according to any one of paras 3 to 7 or 10 to 17, wherein the ADAMTS14 inhibitor reduces gene and/or protein expression of ADAMTS14.

20. The ADAMTS14 inhibitor for use according to any one of paras 1, 4 to 7, 8, 11 to 14, or 19, the use according to any one of paras 2, 4 to 7, 9, 11 to 14, or 19, or the method according to any one of paras 3 to 7 or 10 to 19, wherein the ADAMTS14 inhibitor is an inhibitory nucleic acid selected from: an siRNA, dsiRNA, miRNA, shRNA, pri-miRNA, pre-miRNA, saRNA, snoRNA, and an antisense oligonucleotide.

The present disclosure includes the combination of the aspects and preferred features described except where such a combination is clearly impermissible or expressly avoided.

The section headings used herein are for organisational purposes only and are not to be construed as limiting the subject-matter described.

Aspects and embodiments of the present disclosure will now be illustrated, by way of example, with reference to the accompanying figures. Further aspects and embodiments will be apparent to those skilled in the art. All documents mentioned in this text are incorporated herein by reference.

Throughout this specification, including the claims which follow, unless the context requires otherwise, the word ‘comprise,’ and variations such as ‘comprises’ and ‘comprising,’ will be understood to imply the inclusion of a stated integer or step or group of integers or steps but not the exclusion of any other integer or step or group of integers or steps.

As used in the specification and the appended claims, the singular forms ‘a,’ ‘an,’ and ‘the’ include plural referents unless the context clearly dictates otherwise. Ranges may be expressed herein as from ‘about’ one particular value, and/or to ‘about’ another particular value. When such a range is expressed, another embodiment includes from the one particular value and/or to the other particular value. Similarly, when values are expressed as approximations, by the use of the antecedent ‘about,’ it will be understood that the particular value forms another embodiment.

Where a nucleic acid sequence is disclosed or referred to herein, the reverse complement thereof is also expressly contemplated.

Methods described herein may preferably performed in vitro. The term ‘in vitro’ is intended to encompass procedures not performed with, in or on multi-cellular organisms (e.g. procedures not performed with, in or on the human or animal body), and include methods procedures performed with cells in culture. The term ‘in vivo’ is intended to encompass procedures with, in or on with intact multi-cellular organisms (e.g. procedures performed with, in or on the human or animal body).

### BRIEF DESCRIPTION OF THE FIGURES

Embodiments and experiments illustrating the principles of the present disclosure will now be discussed with reference to the accompanying figures.

FIGS. 1A to 1D. Images and bar charts showing that siRNA-mediated depletion of ADAMTS14 in NHLFs significantly reduces nuclear localization of YAP, and also significantly reduces expression of pro-fibrotic genes induced by TGFβ1. (1A) Fluorescence microscopy images and (1B) quantification of the percentage of cells having YAP staining localised to the nucleus, in NHLFs treated with control, non-targeting siRNA (siNT2) or siADAMTS14, in the presence or absence of stimulation with TGFβ1. (1C) The results of qPCR analysis of ADAMTS14 expression in NHLFs treated with control, non-targeting siRNA (siNT2) or siADAMTS14, in the presence or absence of stimulation with TGFβ1. (1D) Expression of pro-fibrotic genes CTGF, CYR61, COL1A1 by NHLFs treated with control, non-targeting siRNA (siNT2) or siADAMTS14, in the presence or absence of stimulation with TGFβ1, as determined by qPCR.

FIGS. 2A to 2C. Images and bar charts showing that treatment of NHLFs with siADAMTS14 in vitro significantly reduces the level of YAP/TAZ protein, and increases phosphorylation at YAP^{S397} and TAZ^{S89} sites. (2A) Western blot and (2B) quantification of total YAP/TAZ protein level, and (2C) quantification of phosphorylated YAP/TAZ protein level (as a proportion of total YAP/TAZ), following treatment of NHLFs with control siRNA (siNT2), siADAMTS14 or siADAMTS2, in the presence or absence of stimulation with TGFβ1.

FIGS. 3A to 3D. Images and bar charts showing that treatment of NHLFs with siADAMTS14 in vitro significantly reduces p-SMAD2 protein level, and inhibits TGFβ1-induced localization of SMAD2 to the nucleus. (3A) Western blot showing protein expression of p-SMAD2 and SMAD2, and (3B) quantification of the p-SMAD2 (as a proportion of total SMAD2), following treatment of NHLFs with control siRNA (siNT2), siADAMTS14 or siADAMTS2, in the presence or absence of stimulation with TGFβ1. (3C) Fluorescence microscopy images and (3D) quantification of SMAD2 localised to the nucleus following treatment of NHLFs with control siRNA (siNT2), siADAMTS14 or siADAMTS2, in the presence or absence of stimulation with TGFβ1.

FIGS. 4A and 4B. Images and bar chart showing that mRNA expression of ADAMTS14 is significantly higher in a lung biopsy sample from a patient with IPF compared to a lung biopsy sample from a normal lung. (4A) RNAscope® staining for ADAMTS14 mRNA, and (4B) quantification of number of ADAMTS14 mRNA signals per field.

FIGS. 5A and 5B. Bar charts showing that treatment of human lung fibroblasts with siADAMTS14 in vitro significantly reduces TGFβ1-stimulated (5A) formation of α-SMA fibers, and (5B) collagen I deposition, compared to TGFβ1 stimulated, non-siRNA treated (TGFβ1) and non-stimulated, non-siRNA treated control (CTR) cells. Data are presented ±SD of n=3 donors.

FIGS. 6A and 6B. Bar charts showing that treatment of lung fibroblasts with siADAMTS14 in co-culture with SAECs in vitro significantly reduced the expression of pro-fibrotic genes. (6A) Expression of pro-fibrotic genes ADAMTS14, ACTA2, COL1A1 and FN1 by NHLFs treated with control, non-targeting siRNA (siNT2) or siADAMTS14, in the presence or absence of stimulation with IPF-RC, as determined by qPCR. (6B) Expression of metaplastic differentiation/pro-fibrotic genes FOXJ1, KRT5, MUC5AC, MUC5B, SCGB1A1, CTGF, MMP7 and SOX2 by SAECs treated with control, non-targeting siRNA (siNT2) or siADAMTS14, in the presence or absence of stimulation with IPF-RC, as determined by qPCR. N=4 NHLFs/SAECs donors, *p<0.05, **p<0.01, ***p<0.001, Kruskal-Wallis/Dunn's.

FIG. 7. Graph showing that knockout of Adamts14 reduces fibrosis of the lungs in mice, in response to challenge with TGFβ. Wildtype C57Bl/6 mice (WT) or Adamts14−/− mice (KO) were administered AAV-TGFb, and collagen content of the lungs was evaluated 21 days later by analysis of total hydroxyproline content. *p<0.05.

### EXAMPLES

In the present Examples the inventors demonstrate that siRNA-mediated knockdown of expression of ADAMTS14 in lung fibroblasts reduces the level of YAP, inhibits nuclear localization of YAP and promotes phosphorylation and degradation of YAP. Knockdown of ADAMTS14 expression in lung fibroblasts also reduces the level of TAZ, inhibits its nuclear localization and thereby its activity. siRNA-mediated knockdown of expression of ADAMTS14 in lung fibroblasts is also shown to inhibit TGFβ1-mediated signalling via reducing phosphorylation of SMAD2 and localization of SMAD2 to the nucleus. ADAMTS14 expression is found to be elevated in IPF lung tissue compared to healthy control lung tissue. Inhibition of expression of ADAMTS14 in lung fibroblasts is demonstrated to inhibit the expression of pro-fibrotic and metaplastic differentiation genes in response to stimulation with proinflammatory cytokines, and to inhibit formation of α-SMA fibers in and collagen I deposition by human lung fibroblasts in response to stimulation with IPF-RC.

### Example 1: Materials and Methods

### Primary Human Cell Lines

Primary human lung fibroblasts (HLF) from IPF or healthy control donors were collected through the MGH Fibrosis Translational Research program from de-identified discarded excess tissue from clinically indicated surgical lung resections or lung transplant explants. Cells were routinely grown in DMEM (Lonza) supplemented with 10% FBS (Lonza), 2 mM L-Glutamine (Lonza), 100 U/ml penicillin and 100 μg/ml streptomycin (Lonza) in a humidified incubator with 5% CO₂ at 37° C. Unless otherwise stated, experiments were conducted using fibroblasts from healthy control donor 699.

For the Scar in a Jar (SiaJ) and co-culture assays fibroblasts from Lonza (NHLF CC-2512, and DHLF-IPF CC-7231) were used. Fibroblasts were cultured in FGM-2 Fibroblast Growth Medium-2 kit (Lonza CC-3132) in a humidified incubator with 5% CO₂ at 37° C.

Primary siRNA Screen

The siRNA screen and subsequent high-throughput validation experiments were conducted at the ICCB-Longwood Screening Facility at Harvard Medical School. A total of 7,638 siRNA pools from the druggable genome libraries Human 6 and Human 7 (Dharmacon siGENOME siRNA) were screened in triplicate. Non-targeting siRNA control and transfection controls were added to each plate.

Primary human lung fibroblasts (HLF) from a healthy donor were reverse transfected at passage 4 with 20 μM siRNA and Lipofectamine 2000 (Thermo Fisher Scientific, 11668019). Briefly, 0.05 μL lipofectamine were diluted in 10 μL Opti-MEM (Thermo Fisher Scientific, 31985070), complexed for 5 minutes at room temperature and added to each well of a 384-well clear bottom black microplate (Corning, 3764). Then, 1 μL siRNA (1 mM) was added to each well and complexed for 20-30 minutes at room temperature. Finally, 300 cells were added to each well in 40 μL DMEM (Lonza) supplemented with 12.5% FBS (Lonza) and 2 mM L-Glutamine (Lonza). Plates were placed in a humidified incubator with 5% CO₂ at 37° C. for 72 h and then processed for YAP imaging and localization analysis [20].

Secondary siRNA Screen

For further validation of the hits the four individual siRNA duplexes comprising each siRNA pool were tested in the same assay (1,512 siRNA duplexes total). Targets with at least two independent active siRNA duplexes were selected for a validation assay. The same four individual siRNA duplexes were retested for their ability to displace YAP and tested for their ability to reduce YAP target gene expression (CTGF and CYR61). Cell lysates were prepared directly on 384-well plates and gene expression analyzed using a Cells-to-CT 1-Step TaqMan Kit (Thermo Fisher Scientific, A25602), Taqman assays ((Thermo Fisher Scientific) and a QuantStudio 7 Flex Real-Time PCR System (Thermo Fisher Scientific). Gene expression was calculated using the 2^{ΔΔCT} method and β2-microglobulin (B2M) as a housekeeping control. Results were filtered based on ability to reduce CTGF and CYR61 expression, ability to reduce nuclear/cytoplasmic YAP ratio, gene expression in HLF (based on public and internal datasets), target novelty, druggability and safety. The validation assay was repeated with the inclusion of three additional siRNA duplexes (Ambion) and the analysis of target gene expression knockdown by RT-qPCR. Two siRNA duplexes for each of the remaining hits were selected for testing in the TGFβ1 assay. Briefly, HLF were reverse transfected as previously and, after two days, treated with or without 5 ng/mL TGFβ1 in serum-free medium for two days. Plates were prepared for imaging (YAP and actin) and RT-qPCR analysis of pro-fibrotic gene expression (CTGF, CYR61, COL1A1, ACTA2).

### Immunofluorescence Staining

Cells were fixed and stained as previously described [20]. Briefly, cells were fixed with 4% paraformaldehyde (PFA) (Electron Microscopy Sciences, 15710S) for 10 to 15 minutes and blocked for 1 hour at room temperature with blocking solution: 10% goat serum, 0.1% Triton-X in PBS. Primary antibodies were incubated overnight at 4° C. and secondary antibodies were incubated for 1 hour at room temperature in blocking solution. The following antibodies and dilutions were used: YAP (Santa Cruz Biotechnology, sc-101199), 1:500; SMAD2 (Cell Signaling Technologies, 5339S), 1:1000; Rhodamine Phalloidin (Invitrogen, R415), 1:200; Alexa Fluor 488 goat anti-mouse (Thermo Fisher Scientific, A11001), 1:400; Alexa Fluor 647 goat anti-rabbit (Thermo Fisher Scientific, A-21244), 1:400. Cells in 384 and 96 well pates were stained with DAPI (Sigma-Aldrich, 100 ng/mL in PBS) and imaged with the ImageXpress Confocal system (Molecular Devices).

### RNAscope® Staining

Staining was performed following manufacturer's instructions for RNAScope® Multiplex Fluorescent V2 Assay kit. Dilutions of probes used: RNAscope® customize designed probe ADAMTS14-C3 1:750, Opal™ 690 reagent 1:500. Images were taken using Zeiss Axio Imager microscope and the following exposure time: DAPI: 20 ms; Cy5: 300 ms

Cell Culture and siRNA Transfection

Cells were routinely plated at 5×10³ cells/cm² and recombinant human TGFβ1 (R&D systems, 240-B) was used at 5 ng/mL in serum-free medium, unless otherwise stated. For knockdown of gene expression, cells were reverse transfected with Lipofectamine 2000 (Thermo Fisher Scientific, 11668019) and 20 μM siRNA according to the manufacturer's instructions. The following siRNAs from Dharmacon were used: Non-Targeting siRNA Pool #2 (D-001206-14); siGENOME Human ADAMTS14 siRNAs (D-005765-01, D-005765-03, 10 μM each). After 48 h, cells were washed twice and treated with or without TGFβ1 for an additional 24 h in serum-free medium and then processed for gene expression analysis.

### Western Blotting

Protein extracts were prepared by lysing cells in RIPA buffer (Cell Signaling Technology) supplemented with Halt Protease and Phosphatase Inhibitor Cocktail (Thermo Fisher Scientific, 78440) on ice. The protein concentration in each sample was determined using the Pierce BCA Protein assay kit (Thermo Fisher Scientific, 23225) according to the manufacturer's instructions. Proteins were separated by SDS-PAGE (Novex® Bis-Tris 4-12% gels) and transferred to nitrocellulose membranes (Thermo Fisher Scientific, LC2001) using the NuPAGE® Novex® system (Thermo Fisher Scientific), under reducing conditions. Membranes were blocked with and all antibodies were diluted in 5% nonfat milk in TBST. Primary antibodies were incubated overnight at 4° C. and secondary antibodies were added for 30 min at room temperature. Blots were imaged using Odyssey® CLx Imaging System. The following antibodies from Cell Signaling Technology were used at 1:1000: YAP (4912); phospho-YAP (Ser397) (13619); phospho-YAP (Ser127) (4911); TAZ (4883); phospho-TAZ (Ser89) (59971); GAPDH (2118); The following secondary antibodies were used: IRDye® 8000W Goat anti-Rabbit IgG Secondary Antibody (LI-COR, 926-32211), 1:5000; IRDye® 680RD Goat anti-Mouse IgG Secondary Antibody (LI-COR, 926-68070), 1:5000. Images were analyzed with Image Studio™ Software.

RNA Extraction and qPCR

Cell lysates were prepared directly on the plates and gene expression analyzed using a Cells-to-CT™ 1 Step TaqMan™ Kit (Thermo Fisher Scientific, A25602). Quantitative real-time PCR was performed using a CFX Connect Real-Time PCR Detection System (Bio-Rad). The following Taqman assays from Thermo Fisher Scientific were used: CTGF Hs00170014_m1 (FAM-MGB); CYR61 Hs00155479_m1 (FAM-MGB); ACTA2 Hs00426835_g1 (FAM-MGB); COL1A1 Hs00164004_m1 (FAM-MGB); ADAMTS14 Hs01548440_m1 (FAM-MGB); B2M Hs00187842_m1 (FAM-MGB). Gene expression was calculated using the 2^{-ΔΔ}Ct method and β2-microglobulin (B2M) as a housekeeping control.

### Scar in a Jar Assay

Normal (2 donors) and IPF-derived (1 donor) lung fibroblasts were from Lonza. siADAMTS14 was from Dharmacon (Smart Pool L-00576-00-0005). Fibroblasts were trypsinized (Reagent Pack Subculture, Lonza CC-5034) and seeded at a density of 2000 cells/55 μl FGM-2 per well in CellCarrier-384 ultra PDL coated plates (Gibco 6057508). After 5 h, 15 μl per well siADAMTS14/RNAiMAX (ThermoFisher 13778150) mix was added and incubated for 24 h. Final concentration for siRNA was 16 nM. The medium was changed to 60 μl Fibroblast Basal Medium (Lonza CC-3131) and incubated at 37° C., 5% CO₂. After a total of 24 h starvation the cells were ‘crowded’ by changing the medium to 40 μl FBM supplemented with 100 μM L-Ascorbic acid 2-phosphate sesquimagnesium salt hydrate (Sigma A8960), 37.5 mg/mL 70 kDa Ficoll (Sigma F2878) and 25 mg/mL 400 kDa Ficoll (Sigma F2637). TGF-β1 (R&D Systems 240-B) at a final concentration of 5 ng/ml was added together with the crowding medium. After addition of TGF-β1 the cells were incubated at 5% CO₂ and 37° C. for 72 h.

Cells were washed with cold PBS and fixed with methanol for 30 minutes on ice. Fixed cells were washed and permeabilized with PBS-T (1% TritonX 100) for 20 min at RT. Cells were then washed and blocked in 3% BSA in PBS for 30 min at RT. After another washing step cells were incubated with primary antibodies (anti collagen Type I (1:1000 Sigma SAB4200678) and anti-αSMA (1:1000 Sigma A2547) for 1.5 h at 37° C. followed by washing three times with PBS. Secondary antibodies (1:1000, Goat anti-mouse IgG1 Alexa Fluor 568, Invitrogen A-21124 and goat anti-mouse IgG2a Alexa Fluor 647 Sigma SAB4600355) in PBS+Hoechst 33342 (1:2000 ThermoFisher 62249) were incubated for 30 min at 37° C. in blocking buffer. Afterwards samples were washed three times with PBS. CellMask Green (Invitrogen H32714) was added to the wells 1:50000 in PBS (20 μl) and incubated for 30 min at RT.

For High Content Imaging, the Opera Phenix High-Content Screening System (Perkin Elmer) equipped with spinning disk confocal imaging optics was used. For fluorescence imaging 405, 488, 568 and 647 nm lasers were used. Images were stored and analyzed in in Columbus 2.7 Image Data Storage and Analysis System (Perkin Elmer)

### SAECs/NHLFs Co-Culture Assay

30 000 small airway epithelial cells (SAECs, Lonza CC-2547S) were seeded on collagen type I (Corning) coated 6.5 mm Transwell (Corning, 0.4 μm pore size, PET membrane, 3470) in PneumaCult-Ex Plus medium until confluent. Apical medium was then removed and SAECs were stratified and differentiated at air-liquid interface in Pneumacult-ALI-S medium (Stemcell Technologies, 05099) for 28 days. The basolateral medium was exchanged every other day. 25 000 normal human lung fibroblasts (NHLFs, Lonza CC-2512) per well (24-well plate, Corning, 3526) were seeded. After reaching 50-70% confluency, cells were starved overnight and medium was replaced by fresh DMEM before experiment start. After full stratification and differentiation (28 days post ALI), SAECs were brought together with NHLFs and treatments were applied basolaterally for 72 h. Where indicated, IPF-relevant cytokine cocktail (IPF-RC as described in Schruf et al. FASEB J. (2020) 34(6):7825-7846; 1:100 dilution) treatment was applied to SAECs cultures in basolateral medium after 14 days post ALI, when cilia were detected.

### Statistical Analysis

Data was plotted and analyzed for statistical significance using GraphPad Prism 9 software. Two groups were compared using 2-tailed unpaired t-tests and three or more groups were compared using 1-way or 2-way ANOVA with post-hoc tests to correct for multiple comparisons, as indicated in figure legends.

### Example 2: Results and Discussion

### 2.1 ADAMTS14 Depletion Significantly Reduces Nuclear YAP Localization and Inhibits TGFβ1 Induced Fibroblast Activation

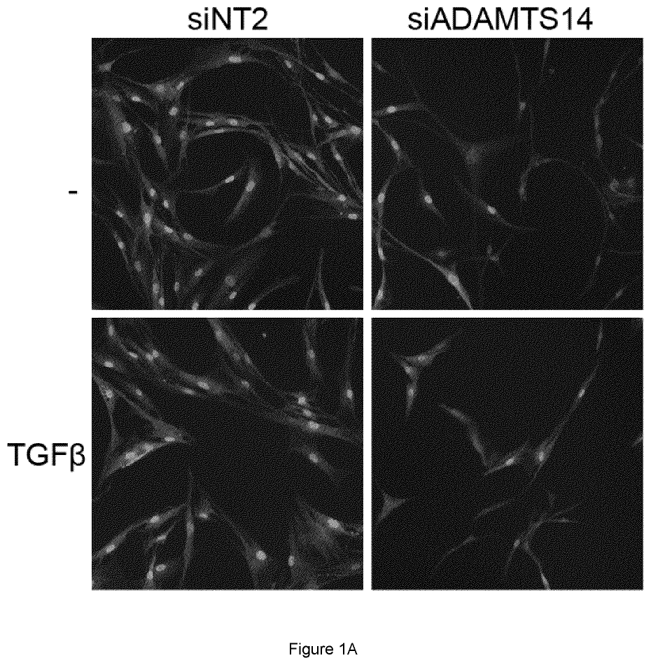

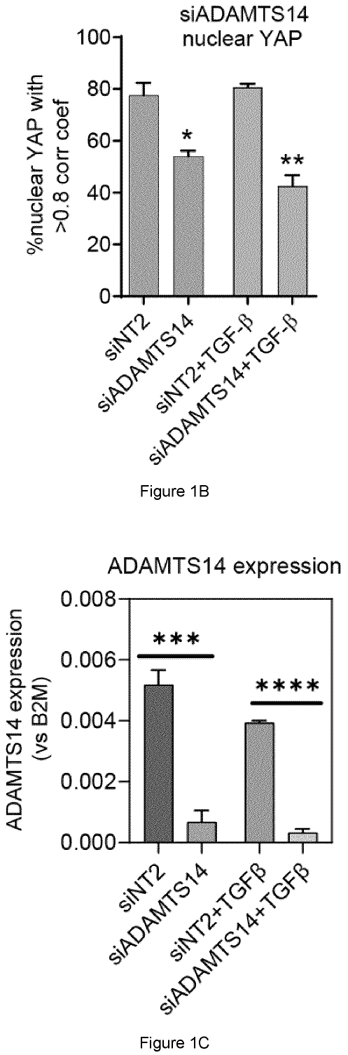

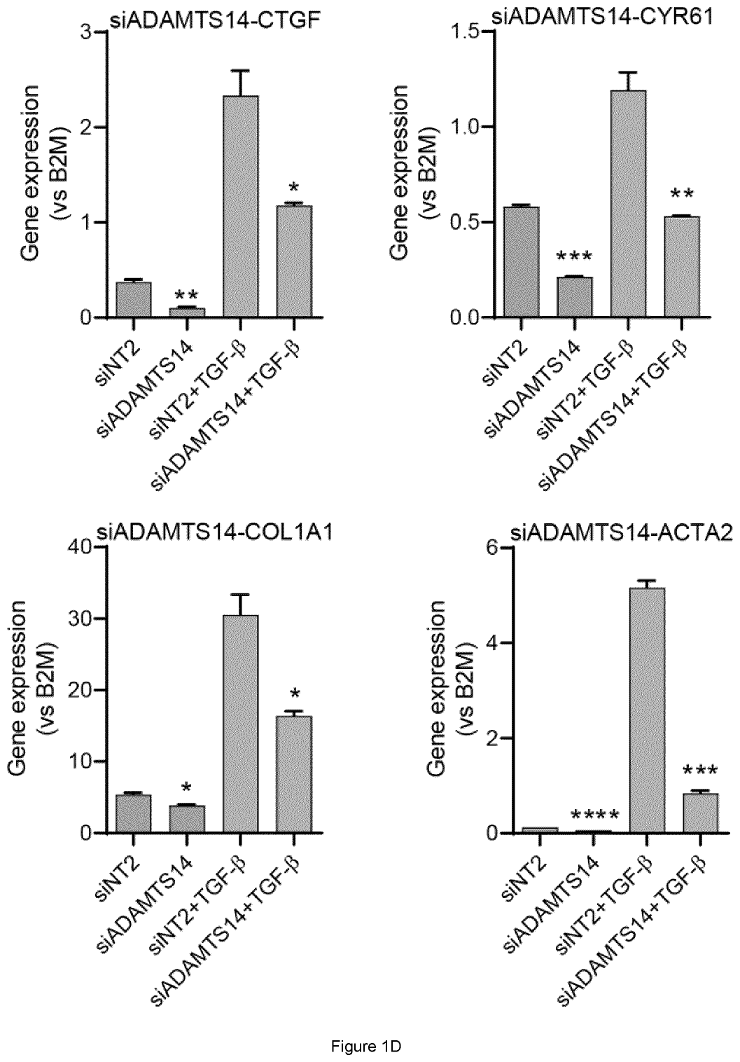

The inventors sought to investigate whether siRNA-mediated knockdown of ADAMTS14 using siADAMTS14 was associated with anti-fibrotic effects in normal human lung fibroblasts (NHLFs). Cells were cultured in 96 well plates in triplicates and reverse transfected with 20 μM siNT2 (siNon-Targeting control #2), or siADAMTS14 (Dharmacon siGENOME individual #1 and #3, 10 μM each). After 48 h, cells were washed twice, and fresh serum free medium was added, with or without 5 ng/ml TGFβ1. After 24 h, cells were fixed in 4% PFA for staining, or prepared for qPCR. The results show that ADAMTS14 expression was efficiently depleted (FIG. 1C), and that siADAMTS14 significantly reduced the percentage of cells with nuclear YAP localization (FIGS. 1A, 1B) and decreased TGFβ1-induced pro-fibrotic gene expression (FIG. 1D).

### 2.2 Depleting ADAMTS14 Leads to YAP/TAZ Phosphorylation and Degradation

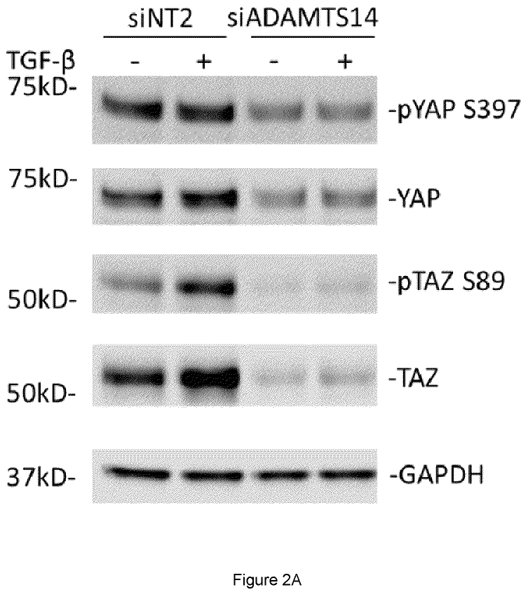

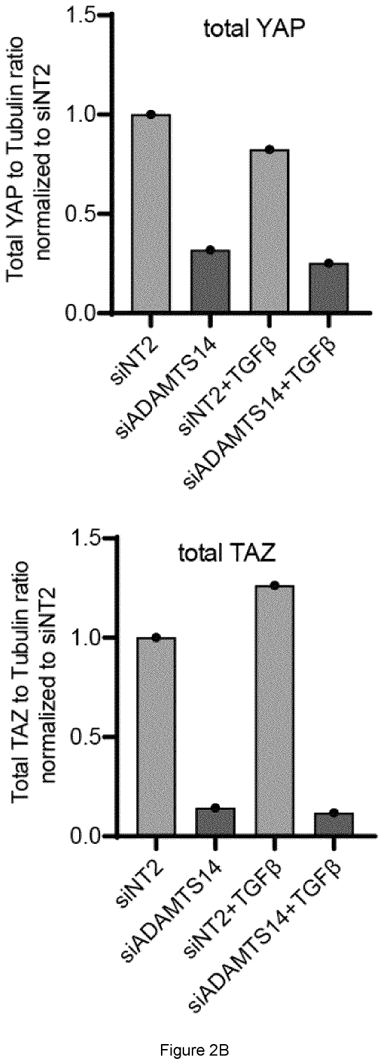

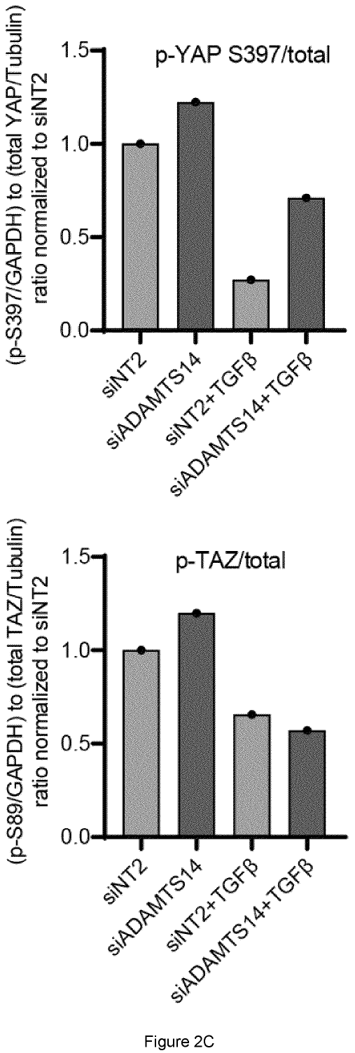

After confirming that ADAMTS14 depletion leads to YAP/TAZ inactivation, we wanted to determine the level and phosphorylation status of YAP/TAZ proteins. Western blots show that total YAP protein level was dramatically decreased after siADAMTS14 (FIGS. 2A, 2B). Consistently, phosphorylation on serine 397 of YAP, which marks YAP for degradation in the cytoplasm, increased after siADAMTS14 (FIG. 2C). TAZ protein level was also dramatically decreased when ADAMTS14 was depleted, with an increase of phosphorylation on serine 89 of TAZ in the absence of TGFβ1, but not when TGFβ1 is added (FIGS. 2A, 2C). Phosphorylation of serine 89 of TAZ has been shown to result in sequestration of TAZ in the cytoplasm, and inhibition of YAP/TAZ activity (Freeman and Morrison, Semin. Cell Dev. Biol. (2012) 23: 681-687; Irvine et al., Semin. Cell Dev. Biol. (2012) 23: 812-817). These results suggest that ADAMTS14 may have a role in regulating the phosphorylation, sequestration and degradation of YAP and TAZ.

2.3 siADAMTS14 Inhibits TGFβ1 Signaling Pathway Through Decreasing SMAD2 Phosphorylation

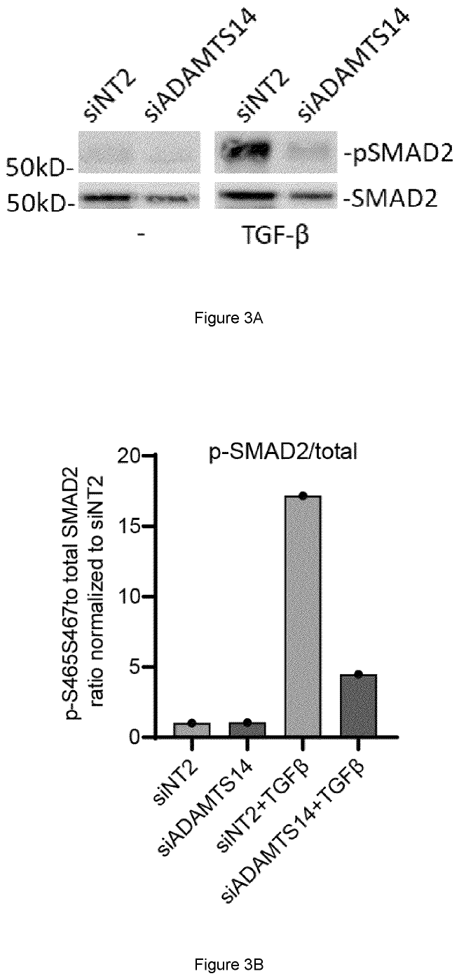

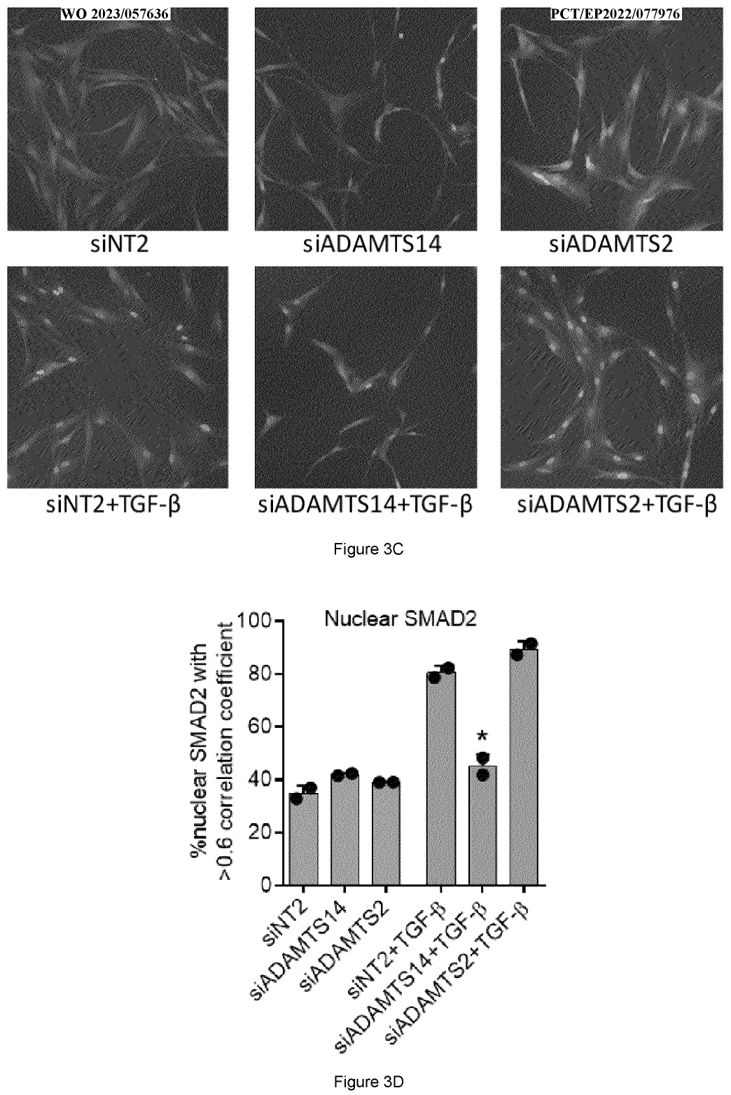

Because ADAMTS14 depletion has a dramatic effect in decreasing TGFβ1-induced expression of pro-fibrotic genes, we next sought to see if canonical TGFβ1 signaling is impaired. TGFβ1 binding to its receptor leads to the activation of the receptor, which phosphorylates SMAD2 on serine residues 465 and 467. Phosphorylated SMAD2 can go into the nucleus and promote target gene expression. NHLFs were plated in 6 well plates at 5×10⁴ cells per well, and reverse transfected with 20 μM siRNA or siNT2. After 48 h, cells were washed twice, and fresh serum free medium was added with or without 5 ng/ml TGFβ1. After another 24 h, cell lysates were directly harvested by lysing with RIPA buffer in the wells and resolved using SDSPAGE. Western blot result showed that the phosphorylated form of SMAD2 was significantly decreased in HLFs after ADAMTS14 knockdown (FIGS. 3A, 3B). Fluorescent imaging of SMAD2 confirmed that the percentage of cells having SMAD2 localized in the nucleus was significantly decreased after siADAMTS14 (FIGS. 3C, 3D). These data suggest that ADAMTS14 has a role in promoting TGFβ1 canonical signaling through SMAD2 phosphorylation.

Interestingly, ADAMTS2 depletion did not have the same effect on TGFβ1 signaling as siADAMTS14, as SMAD2 remained highly enriched in the nucleus when TGFβ1 was added to the cells (FIGS. 3C, 3D). This result shows that although ADAMTS2 and ADAMTS14 are co-expressed and share similar pools of substrates, they may have distinct roles in regulating specific signaling pathways. In an Adamts2 and Adamts14 double knockout mouse model, there were some skin lesion phenotypes that were not present in the Adamts2 single knockout mouse [21], which suggests that ADAMTS14 may have some unknown additional roles.

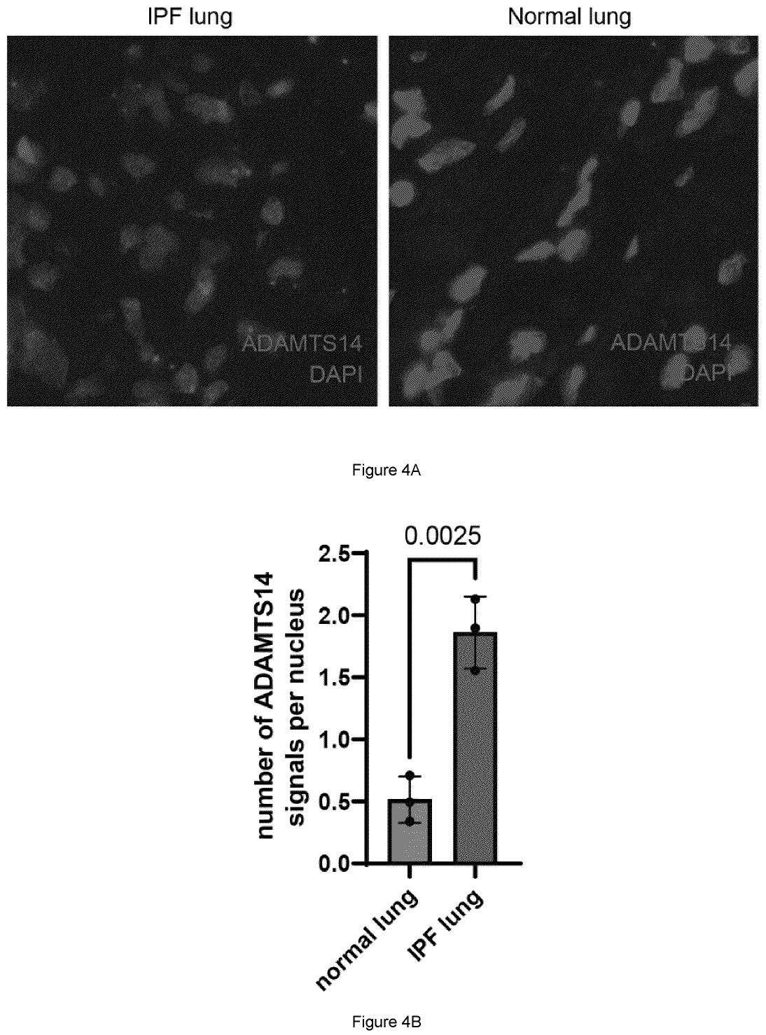

2.4 ADAMTS14 mRNA Expression is Significantly Higher in IPF Lung Biopsy Compared to Normal Lung IPF lung biopsy containing the fibroblast foci were prepared by embedding in paraffin, and microtome cutting and mounting onto slides. Using a custom designed ADAMTS14 RNAscope® probe, we were able to detect significantly higher numbers of ADAMTS14 signal per field of fibroblast foci in IPF lung compared to normal lung (FIGS. 4A, 4B). This is the first evidence showing that ADAMTS14 is elevated in IPF fibroblast foci and could potentially be targeted by treatment.

### 2.5 ADAMTS14 Depletion Significantly Inhibits TGFβ1 Induced αSMA Fiber Formation and Collagen I Deposition

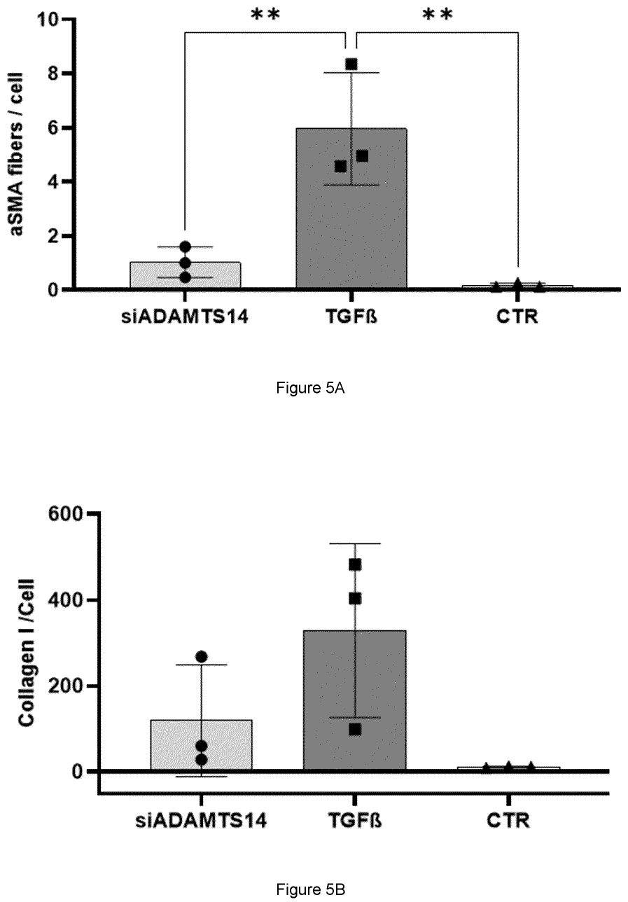

To determine the inhibitory potency of a siRNA knock down of ADAMST14 on αSMA fiber formation and Collagen I deposition, two hallmarks of pro-fibrotic fibroblast-to-myofibroblast transition (FMT), data from 2 healthy and 1 IPF-derived lung fibroblast lines were collected. siADAMTS14 significantly inhibited TGFβ-induced α-SMA fiber formation by 82.2% (FIG. 5A). Furthermore, Collagen I deposition by lung fibroblasts was reduced by 71.2% compared to TGFβ plus transfection reagent only treated cells (FIG. 5B).

### 2.6 ADAMTS14 Depletion in Fibroblasts Attenuates Metaplastic Differentiation of the Airway Epithelium

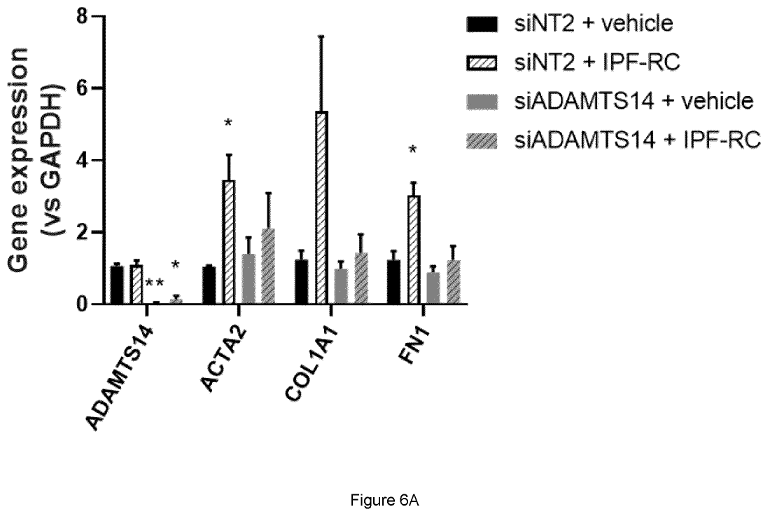

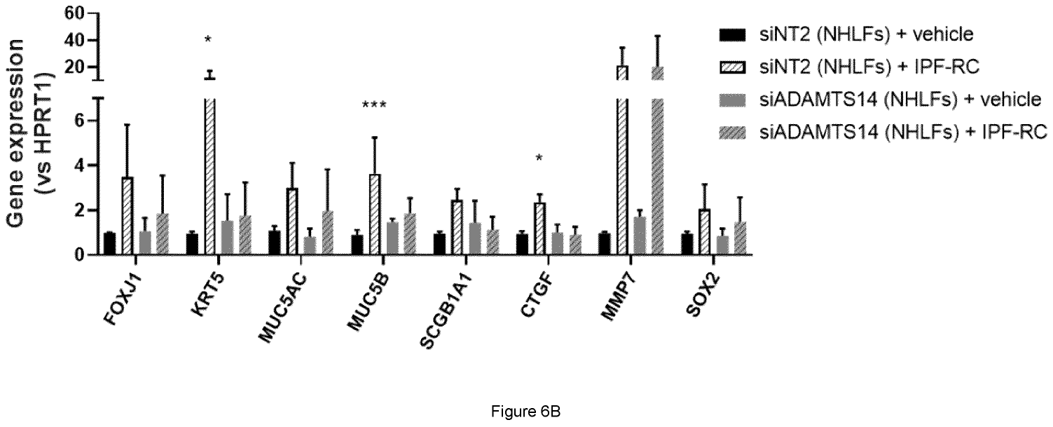

As fibroblasts have been shown to promote epithelial remodeling upon injury, we next assessed whether ADAMTS14 depleted fibroblasts in turn influence small airway epithelial cells (SAECs) during fibrotic repair. To test this, we employed an air-liquid interface (ALI) culture of SAECs in co-culture with either non-targeting siRNA (siNT2) or ADAMTS14 knockdown (siADAMTS14) fibroblasts. We further exposed the co-culture system to a pro-fibrotic environment stimulated by an IPF-relevant cocktail (IPF-RC) for 72 h [22]. We first observed an induction of FMT process in NHLFs upon IPF-RC treatment (FIG. 6A). As expected, depletion of ADAMTS14 attenuated IPF-RC induced FMT. These results reinforce our observations that ADAMTS14 plays important roles in fibrotic progression in fibroblasts. Furthermore, analysis of the airway epithelium revealed a dramatic increase in the expression of metaplasia/pro-fibrotic genes upon IPF-RC treatment (FIG. 6B). Importantly, ADAMTS14-depleted fibroblasts attenuated the fibrotic effect of IPF-RC on airway epithelial lineage commitment genes. These data suggest that ADAMTS14 depleted fibroblasts, in conjunction with pro-fibrotic multi-factorial stimuli, acts as an anti-fibrotic niche to prevent aberrant epithelial remodeling upon injury.

### Example 3: Adamts14 Knockout Mice Display Less Fibrosis In Vivo Compared to Wildtype Mice in Response to a Pro-Fibrotic Stimulus

In experiments, the inventors investigated the fibrotic response in wildtype mice and mice engineered to knockout expression of Adamts14, following treatment with a pro-fibrotic stimulus.

Codon-usage-optimised murine cDNA encoding Tgfb1 was cloned into an AAV2-inverted terminal repeat (ITR)- and cytomegalovirus (CMV) promoter-containing pAAV vector. The AAV6.2 cap gene (accession no. EU368910.1) was used to replace the AAV2 cap sequence in pAAV-RC. AAV6.2 vectors were produced by calcium phosphate transfection of human embryonic kidney (HEK)-293 cells using a three-plasmid based production protocol (AAV helper free system; no. 240071; Agilent Technologies, Waldbronn, Germany) and purified. Virus titer: AAV6.2-TGFb1: 7.26e+13 vg/mL.

Under light anaesthesia (3-4% isoflurane), adult male wildtype C57Bl/6 mice or Adamts14−/− mice were administered 50 μl of 2.7×10¹¹ vg AAV6.2-TGFb1 virus suspension into the trachea using Endotracheal Intubation Kit (Kent Scientific). After 21 days, mice were sacrificed, and their lungs harvested for fibrosis assessment.

Total lung hydroxyproline content was determined for assessment of lung fibrosis. Briefly, collected lungs were flash-frozen and stored at −80° C. until analysed. Lungs were then thawed on ice, homogenized in PBS with Halt™ Protease and Phosphatase Inhibitor (Thermo Fisher Scientific, 78440) and hydrolysed overnight in 6N HCl at 120° C. Next, 25 μL aliquots were added to 1 ml of 1.4% chloramine T (MilliporeSigma), 10% n-propanol, and 0.5 M sodium acetate, pH 6.0 and incubated at room temperature for 20 min. Then, 1 ml of Erlich's solution (1 M p-dimethylaminobezaldehyde (MilliporeSigma) in 70% n-propanol, 20% perchloric acid) was added and samples were incubated a 65° C. for 15 minutes. Absorbance was measured at 550 nm and the amount of hydroxyproline was determined against a standard curve.

Data were plotted and analysed for statistical significance using GraphPad Prism software. Two groups were compared using 2-tailed t-tests.

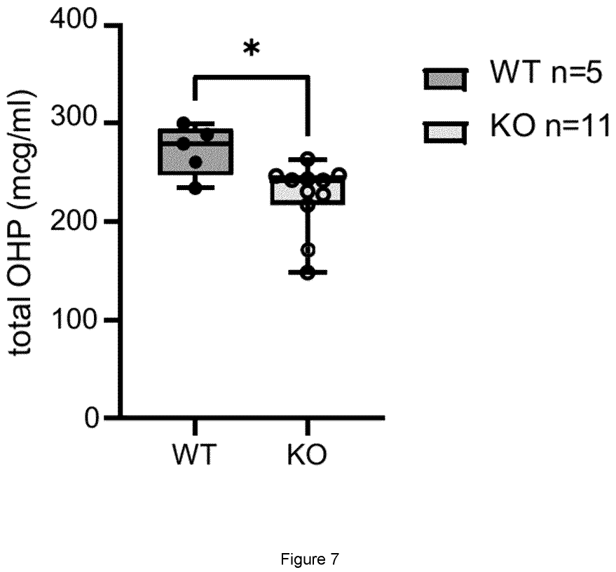

The results are shown in FIG. 7. Adamts14 knockout mice displayed statistically-significantly less fibrosis of the lung compared to wildtype mice following treatment with AAV-TGFb, as determined by detection of reduced total hydroxyproline content.

### REFERENCES

1. Raghu, G., et al., An Official ATS/ERS/JRS/ALAT Clinical Practice Guideline: Treatment of Idiopathic Pulmonary Fibrosis. An Update of the 2011 Clinical Practice Guideline. Am J Respir Crit Care Med, 2015. 192(2): p. e3-19.
2. King, T. E., Jr., et al., A phase 3 trial of pirfenidone in patients with idiopathic pulmonary fibrosis. N Engl J Med, 2014. 370(22): p. 2083-92.
3. Richeldi, L., et al., Efficacy and safety of nintedanib in idiopathic pulmonary fibrosis. N Engl J Med, 2014. 370(22): p. 2071-82.
4. Kanai, F., et al., TAZ: a novel transcriptional co-activator regulated by interactions with 14-3-3 and PDZ domain proteins. EMBO J, 2000. 19(24): p. 6778-91.
5. Yagi, R., et al., A WW domain-containing yes-associated protein (YAP) is a novel transcriptional co-activator. EMBO J, 1999. 18(9): p. 2551-62.
6. Dupont, S., et al., Role of YAP/TAZ in mechanotransduction. Nature, 2011. 474(7350): p. 179-83.
7. Liu, F., et al., Mechanosignaling through YAP and TAZ drives fibroblast activation and fibrosis. Am J Physiol Lung Cell Mol Physiol, 2015. 308(4): p. L344-57.
8. Szeto, S. G., et al., YAP/TAZ Are Mechanoregulators of TGF-beta-Smad Signaling and Renal Fibrogenesis. J Am Soc Nephrol, 2016. 27(10): p. 3117-3128.
9. Zhao, B., et al., The Hippo-YAP pathway in organ size control and tumorigenesis: an updated version. Genes Dev, 2010. 24(9): p. 862-74.
10. Colige, A., et al., Cloning and characterization of ADAMTS-14, a novel ADAMTS displaying high homology with ADAMTS-2 and ADAMTS-3. J Biol Chem, 2002. 277(8): p. 5756-66.
11. Bekhouche, M., et al., Determination of the substrate repertoire of ADAMTS2, 3, and 14 significantly broadens their functions and identifies extracellular matrix organization and TGF-beta signaling as primary targets. FASEB J, 2016. 30(5): p. 1741-56.
12. Janssen, L., et al., ADAMTS3 activity is mandatory for embryonic lymphangiogenesis and regulates placental angiogenesis. Angiogenesis, 2016. 19(1): p. 53-65.
13. Brouillard, P., et al., Loss of ADAMTS3 activity causes Hennekam lymphangiectasia-lymphedema syndrome 3. Hum Mol Genet, 2017. 26(21): p. 4095-4104.
14. Wang, G., et al., Specific fibroblast subpopulations and neuronal structures provide local sources of Vegfc-processing components during zebrafish lymphangiogenesis. Nat Commun, 2020. 11(1): p. 2724.
15. Leduc, C., et al., In vivo N-Terminomics Highlights Novel Functions of ADAMTS2 and ADAMTS14 in Skin Collagen Matrix Building. Front Mol Biosci, 2021. 8: p. 643178.
16. Goertsches, R., et al., Genetic association between polymorphisms in the ADAMTS14 gene and multiple sclerosis. J Neuroimmunol, 2005. 164(1-2): p. 140-7.
17. Ma, S., C. Ouyang, and S. Ren, Relationship between ADAMTS14/rs4747096 gene polymorphism and knee osteoarthritis in Chinese population. Biosci Rep, 2018. 38(5).
18. Poonpet, T., et al., ADAMTS14 gene polymorphism associated with knee osteoarthritis in Thai women. Genet Mol Res, 2013. 12(4): p. 5301-9.
19. Rodriguez-Lopez, J., et al., Association of a nsSNP in ADAMTS14 to some osteoarthritis phenotypes. Osteoarthritis Cartilage, 2009. 17(3): p. 321-7.
20. Santos, D. M., et al., Screening for YAP Inhibitors Identifies Statins as Modulators of Fibrosis. Am J Respir Cell Mol Biol, 2020. 62(4): p. 479-492.
21. Dupont, L., et al., Spontaneous atopic dermatitis due to immune dysregulation in mice lacking Adamts2 and 14. Matrix Biol, 2018. 70: p. 140-157.
22. Schruf, E., et al., Recapitulating idiopathic pulmonary fibrosis related alveolar epithelial dysfunction in a human iPSC-derived air-liquid interface model. FASEB J, 2020. 34(6): p. 7825-7846.

1. An ADAMTS14 inhibitor for use in the treatment or prevention of fibrosis.

2. The ADAMTS14 inhibitor for use according to claim 1, wherein the fibrosis is fibrosis of: an organ or tissue of the respiratory system, lung, bronchioles, alveoli, airways, nasal cavity, oral cavity, pharynx, larynx, trachea, bronchus, an organ or tissue of the cardiovascular system, heart, blood vessels, an organ or tissue of the gastrointestinal system, liver, bowel, small intestine, large intestine, colon, pancreas, skin, eye, an organ or tissue of the nervous system, brain, an organ or tissue of the urogenital system, kidney, ovaries, fallopian tubes, an organ or tissue of the musculoskeletal system, muscle tissue, or bone marrow.

3. The ADAMTS14 inhibitor for use according to claim 1 or claim 2, wherein the fibrosis is fibrosis of a disease or condition selected from: pulmonary fibrosis, interstitial lung disease (ILD), idiopathic interstitial pneumonia (IIP), idiopathic pulmonary fibrosis (IPF), cystic fibrosis, progressive massive fibrosis, scleroderma, obliterative bronchiolitis, Hermansky-Pudlak syndrome, asbestosis, silicosis, sarcoidosis, tumor stroma in lung disease, chronic obstructive pulmonary disease (COPD), emphysema, chronic bronchitis, asthma, chronic liver disease, liver fibrosis, cirrhosis, non-alcoholic fatty liver disease (NAFLD), steatohepatitis, non-alcoholic steatohepatitis (NASH), alcoholic liver disease (ALD), alcoholic fatty liver (AFL), alcoholic hepatitis, alcoholic steatohepatitis (ASH), primary biliary cirrhosis (PBC), schistosomal liver disease, hepatocellular carcinoma (HCC), hypertrophic cardiomyopathy (HCM), dilated cardiomyopathy (DCM), fibrosis of the atrium, atrial fibrillation, fibrosis of the ventricle, ventricular fibrillation, myocardial fibrosis, Brugada syndrome, myocarditis, endomyocardial fibrosis, myocardial infarction, fibrotic vascular disease, hypertension, hypertensive heart disease, arrhythmogenic right ventricular cardiomyopathy (ARVC), atherosclerosis, chronic pulmonary hypertension, AIDS-associated pulmonary hypertension, varicose veins, cerebral infarcts, tubulointerstitial fibrosis, glomerular fibrosis, renal fibrosis, nephritic syndrome, Alport's syndrome, HIV-associated nephropathy, polycystic kidney disease, Fabry's disease, diabetic nephropathy, chronic glomerulonephritis, nephritis associated with systemic lupus, pancreatic fibrosis, chronic pancreatitis, endometriosis, gliosis, Alzheimer's disease, multiple sclerosis, muscular dystrophy, Duchenne muscular dystrophy (DMD), Becker's muscular dystrophy (BMD), fibrotic myopathy, inflammatory bowel disease (IBD), Crohn's disease, microscopic colitis, primary sclerosing cholangitis (PSC), scleroderma, nephrogenic systemic fibrosis, Dupuytren's contracture, cutis keloid, Grave's opthalmopathy, epiretinal fibrosis, retinal fibrosis, subretinal fibrosis, subretinal fibrosis associated with macular degeneration, wet age-related macular degeneration (AMD), diabetic retinopathy, glaucoma, corneal fibrosis, post-surgical fibrosis, post-surgical fibrosis of the posterior capsule following cataract surgery, post-surgical fibrosis of the bleb following trabeculectomy for glaucoma, conjunctival fibrosis, subconjunctival fibrosis, arthrofibrosis, arthritis, adhesive capsulitis, progressive systemic sclerosis (PSS), chronic graft versus host disease (GVHD), fibrotic pre-neoplastic disease, fibrotic neoplastic disease, fibrosis induced by chemical insult, or fibrosis induced by environmental insult, fibrosis induced by cancer chemotherapy, fibrosis induced by pesticides, fibrosis induced by radiation, fibrosis induced by cancer radiotherapy, cancer, hepatocellular carcinoma, gastric cancer, esophageal cancer, head and neck cancer, colorectal cancer, pancreatic cancer, cervical cancer, vulvar cancer, mediastinal fibrosis, retroperitoneal fibrosis, myelofibrosis and Peyronie's disease.

4. The ADAMTS14 inhibitor for use according to any one of claims 1 to 3, wherein the fibrosis is fibrosis of: an organ or tissue of the respiratory system, lung, bronchioles, alveoli, airways, nasal cavity, oral cavity, pharynx, larynx, trachea or bronchus.

5. The ADAMTS14 inhibitor for use according to any one of claims 1 to 4, wherein the fibrosis is fibrosis of a disease or condition selected from: pulmonary fibrosis, interstitial lung disease (ILD), idiopathic interstitial pneumonia (IIP), idiopathic pulmonary fibrosis (IPF), cystic fibrosis, progressive massive fibrosis, scleroderma, obliterative bronchiolitis, Hermansky-Pudlak syndrome, asbestosis, silicosis, sarcoidosis, tumor stroma in lung disease, chronic obstructive pulmonary disease (COPD), emphysema, chronic bronchitis and asthma.

6. An ADAMTS14 inhibitor for use in the treatment or prevention of a disease or condition characterised by fibrosis.

7. The ADAMTS14 inhibitor for use according to claim 6, wherein the disease or condition characterised by fibrosis comprises fibrosis of: an organ or tissue of the respiratory system, lung, bronchioles, alveoli, airways, nasal cavity, oral cavity, pharynx, larynx, trachea, bronchus, an organ or tissue of the cardiovascular system, heart, blood vessels, an organ or tissue of the gastrointestinal system, liver, bowel, small intestine, large intestine, colon, pancreas, skin, eye, an organ or tissue of the nervous system, brain, an organ or tissue of the urogenital system, kidney, ovaries, fallopian tubes, an organ or tissue of the musculoskeletal system, muscle tissue, or bone marrow.

8. The ADAMTS14 inhibitor for use according to claim 6 or claim 7, wherein the disease or condition characterised by fibrosis is selected from: pulmonary fibrosis, interstitial lung disease (ILD), idiopathic interstitial pneumonia (IIP), idiopathic pulmonary fibrosis (IPF), cystic fibrosis, progressive massive fibrosis, scleroderma, obliterative bronchiolitis, Hermansky-Pudlak syndrome, asbestosis, silicosis, sarcoidosis, tumor stroma in lung disease, chronic obstructive pulmonary disease (COPD), emphysema, chronic bronchitis, asthma, chronic liver disease, liver fibrosis, cirrhosis, non-alcoholic fatty liver disease (NAFLD), steatohepatitis, non-alcoholic steatohepatitis (NASH), alcoholic liver disease (ALD), alcoholic fatty liver (AFL), alcoholic hepatitis, alcoholic steatohepatitis (ASH), primary biliary cirrhosis (PBC), schistosomal liver disease, hepatocellular carcinoma (HCC), hypertrophic cardiomyopathy (HCM), dilated cardiomyopathy (DCM), fibrosis of the atrium, atrial fibrillation, fibrosis of the ventricle, ventricular fibrillation, myocardial fibrosis, Brugada syndrome, myocarditis, endomyocardial fibrosis, myocardial infarction, fibrotic vascular disease, hypertension, hypertensive heart disease, arrhythmogenic right ventricular cardiomyopathy (ARVC), atherosclerosis, chronic pulmonary hypertension, AIDS-associated pulmonary hypertension, varicose veins, cerebral infarcts, tubulointerstitial fibrosis, glomerular fibrosis, renal fibrosis, nephritic syndrome, Alport's syndrome, HIV-associated nephropathy, polycystic kidney disease, Fabry's disease, diabetic nephropathy, chronic glomerulonephritis, nephritis associated with systemic lupus, pancreatic fibrosis, chronic pancreatitis, endometriosis, gliosis, Alzheimer's disease, multiple sclerosis, muscular dystrophy, Duchenne muscular dystrophy (DMD), Becker's muscular dystrophy (BMD), fibrotic myopathy, inflammatory bowel disease (IBD), Crohn's disease, microscopic colitis, primary sclerosing cholangitis (PSC), scleroderma, nephrogenic systemic fibrosis, Dupuytren's contracture, cutis keloid, Grave's opthalmopathy, epiretinal fibrosis, retinal fibrosis, subretinal fibrosis, subretinal fibrosis associated with macular degeneration, wet age-related macular degeneration (AMD), diabetic retinopathy, glaucoma, corneal fibrosis, post-surgical fibrosis, post-surgical fibrosis of the posterior capsule following cataract surgery, post-surgical fibrosis of the bleb following trabeculectomy for glaucoma, conjunctival fibrosis, subconjunctival fibrosis, arthrofibrosis, arthritis, adhesive capsulitis, progressive systemic sclerosis (PSS), chronic graft versus host disease (GVHD), fibrotic pre-neoplastic disease, fibrotic neoplastic disease, fibrosis induced by chemical insult, or fibrosis induced by environmental insult, fibrosis induced by cancer chemotherapy, fibrosis induced by pesticides, fibrosis induced by radiation, fibrosis induced by cancer radiotherapy, cancer, hepatocellular carcinoma, gastric cancer, esophageal cancer, head and neck cancer, colorectal cancer, pancreatic cancer, cervical cancer, vulvar cancer, mediastinal fibrosis, retroperitoneal fibrosis, myelofibrosis and Peyronie's disease.

9. The ADAMTS14 inhibitor for use according to any one of claims 6 to 8, wherein the disease or condition characterised by fibrosis comprises fibrosis of: an organ or tissue of the respiratory system, lung, bronchioles, alveoli, airways, nasal cavity, oral cavity, pharynx, larynx, trachea or bronchus.

10. The ADAMTS14 inhibitor for use according to any one of claims 6 to 9, wherein the disease or condition characterised by fibrosis is selected from: pulmonary fibrosis, interstitial lung disease (ILD), idiopathic interstitial pneumonia (IIP), idiopathic pulmonary fibrosis (IPF), cystic fibrosis, progressive massive fibrosis, scleroderma, obliterative bronchiolitis, Hermansky-Pudlak syndrome, asbestosis, silicosis, sarcoidosis, tumor stroma in lung disease, chronic obstructive pulmonary disease (COPD), emphysema, chronic bronchitis and asthma.

11. A method of inhibiting TGFβ1-mediated signalling or YAP-mediated signalling in a cell, comprising contacting a cell with an ADAMTS14 inhibitor.

12. A method of inhibiting the generation of a pro-fibrotic fibroblast, comprising contacting a pro-fibrotic fibroblast precursor cell with an ADAMTS14 inhibitor.

13. A method of inhibiting a process mediated by pro-fibrotic fibroblasts, comprising contacting a pro-fibrotic fibroblast or a pro-fibrotic fibroblast precursor cell with an ADAMTS14 inhibitor.

14. The ADAMTS14 inhibitor for use according to any one of claims 1 to 10, or the method according to any one of claims 11 to 13, wherein the ADAMTS14 inhibitor reduces gene and/or protein expression of ADAMTS14.

15. The ADAMTS14 inhibitor for use according to any one of claims 1 to 10 or claim 14, or the method according to any one of claims 11 to 14, wherein the ADAMTS14 inhibitor is an inhibitory nucleic acid selected from: an siRNA, dsiRNA, miRNA, shRNA, pri-miRNA, pre-miRNA, saRNA, snoRNA, and an antisense oligonucleotide.


SEQUENCE LISTING (19 sequences)

SEQ ID NO: 1 - protein, 1223 aa, synthetic construct
  REGION 1..1223: note = Human ADAMTS14 isoform A (UniProtKB: Q8WXS8-1, v2)
MAPLRALLSYLLPLHCALCAAAGSRTPELHLSGKLSDYGVTVPCSTDFRGRFLSHVVSGPAAASAGSMVVDTPPTLPRHSSHLRVARSPLHPGGTLWPGRVGRHSLYFNVTVFGKELHLRLRPNRRLVVPGSSVEWQEDFRELFRQPLRQECVYTGGVTGMPGAAVAISNCDGLAGLIRTDSTDFFIEPLERGQQEKEASGRTHVVYRREAVQQEWAEPDGDLHNEAFGLGDLPNLLGLVGDQLGDTERKRRHAKPGSYSIEVLLVVDDSVVRFHGKEHVQNYVLTLMNIVDEIYHDESLGVHINIALVRLIMVGYRQSLSLIERGNPSRSLEQVCRWAHSQQRQDPSHAEHHDHVVFLTRQDFGPSGYAPVTGMCHPLRSCALNHEDGFSSAFVIAHETGHVLGMEHDGQGNGCADETSLGSVMAPLVQAAFHRFHWSRCSKLELSRYLPSYDCLLDDPFDPAWPQPPELPGINYSMDEQCRFDFGSGYQTCLAFRTFEPCKQLWCSHPDNPYFCKTKKGPPLDGTECAPGKWCFKGHCIWKSPEQTYGQDGGWSSWTKFGSCSRSCGGGVRSRSRSCNNPSPAYGGRLCLGPMFEYQVCNSEECPGTYEDFRAQQCAKRNSYYVHQNAKHSWVPYEPDDDAQKCELICQSADTGDVVFMNQVVHDGTRCSYRDPYSVCARGECVPVGCDKEVGSMKADDKCGVCGGDNSHCRTVKGTLGKASKQAGALKLVQIPAGARHIQIEALEKSPHRIVVKNQVTGSFILNPKGKEATSRTFTAMGLEWEDAVEDAKESLKTSGPLPEAIAILALPPTEGGPRSSLAYKYVIHEDLLPLIGSNNVLLEEMDTYEWALKSWAPCSKACGGGIQFTKYGCRRRRDHHMVQRHLCDHKKRPKPIRRRCNQHPCSQPVWVTEEWGACSRSCGKLGVQTRGIQCLLPLSNGTHKVMPAKACAGDRPEARRPCLRVPCPAQWRLGAWSQCSATCGEGIQQRQVVCRTNANSLGHCEGDRPDTVQVCSLPACGGNHQNSTVRADVWELGTPEGQWVPQSEPLHPINKISSTEPCTGDRSVFCQMEVLDRYCSIPGYHRLCCVSCIKKASGPNPGPDPGPTSLPPFSTPGSPLPGPQDPADAAEPPGKPTGSEDHQHGRATQLPGALDTSSPGTQHPFAPETPIPGASWSISPTTPGGLPWGWTQTPTPVPEDKGQPGEDLRHPGTSLPAASPVT

SEQ ID NO: 2 - protein, 1156 aa, synthetic construct
  REGION 1..1156: note = Human ADAMTS14 isoform B (UniProtKB: Q8WXS8-2)
MVVDTPPTLPRHSSHLRVARSPLHPGGTLWPGRVGRHSLYFNVTVFGKELHLRLRPNRRLVVPGSSVEWQEDFRELFRQPLRQECVYTGGVTGMPGAAVAISNCDGLAGLIRTDSTDFFIEPLERGQQEKEASGRTHVVYRREAVQQEWAEPDGDLHNEAFGLGDLPNLLGLVGDQLGDTERKRRHAKPGSYSIEVLLVVDDSVVRFHGKEHVQNYVLTLMNIVDEIYHDESLGVHINIALVRLIMVGYRQSLSLIERGNPSRSLEQVCRWAHSQQRQDPSHAEHHDHVVFLTRQDFGPSGYAPVTGMCHPLRSCALNHEDGFSSAFVIAHETGHVLGMEHDGQGNGCADETSLGSVMAPLVQAAFHRFHWSRCSKLELSRYLPSYDCLLDDPFDPAWPQPPELPGINYSMDEQCRFDFGSGYQTCLAFRTFEPCKQLWCSHPDNPYFCKTKKGPPLDGTECAPGKWCFKGHCIWKSPEQTYGQDGGWSSWTKFGSCSRSCGGGVRSRSRSCNNPSPAYGGRLCLGPMFEYQVCNSEECPGTYEDFRAQQCAKRNSYYVHQNAKHSWVPYEPDDDAQKCELICQSADTGDVVFMNQVVHDGTRCSYRDPYSVCARGECVPVGCDKEVGSMKADDKCGVCGGDNSHCRTVKGTLGKASKQAGALKLVQIPAGARHIQIEALEKSPHRIVVKNQVTGSFILNPKGKEATSRTFTAMGLEWEDAVEDAKESLKTSGPLPEAIAILALPPTEGGPRSSLAYKYVIHEDLLPLIGSNNVLLEEMDTYEWALKSWAPCSKACGGGIQFTKYGCRRRRDHHMVQRHLCDHKKRPKPIRRRCNQHPCSQPVWVTEEWGACSRSCGKLGVQTRGIQCLLPLSNGTHKVMPAKACAGDRPEARRPCLRVPCPAQWRLGAWSQCSATCGEGIQQRQVVCRTNANSLGHCEGDRPDTVQVCSLPACGGNHQNSTVRADVWELGTPEGQWVPQSEPLHPINKISSTEPCTGDRSVFCQMEVLDRYCSIPGYHRLCCVSCIKKASGPNPGPDPGPTSLPPFSTPGSPLPGPQDPADAAEPPGKPTGSEDHQHGRATQLPGALDTSSPGTQHPFAPETPIPGASWSISPTTPGGLPWGWTQTPTPVPEDKGQPGEDLRHPGTSLPAASPVT

SEQ ID NO: 3 - protein, 1159 aa, synthetic construct
  REGION 1..1159: note = Human ADAMTS14 isoform C (UniProtKB: Q8WXS8-4)
MVVDTPPTLPRHSSHLRVARSPLHPGGTLWPGRVGRHSLYFNVTVFGKELHLRLRPNRRLVVPGSSVEWQEDFRELFRQPLRQECVYTGGVTGMPGAAVAISNCDGLAGLIRTDSTDFFIEPLERGQQEKEASGRTHVVYRREAVQQEWAEPDGDLHNEAFGLGDLPNLLGLVGDQLGDTERKRRHAKPGSYSIEVLLVVDDSVVRFHGKEHVQNYVLTLMNIVDEIYHDESLGVHINIALVRLIMVGYRQSLSLIERGNPSRSLEQVCRWAHSQQRQDPSHAEHHDHVVFLTRQDFGPSGMQGYAPVTGMCHPLRSCALNHEDGFSSAFVIAHETGHVLGMEHDGQGNGCADETSLGSVMAPLVQAAFHRFHWSRCSKLELSRYLPSYDCLLDDPFDPAWPQPPELPGINYSMDEQCRFDFGSGYQTCLAFRTFEPCKQLWCSHPDNPYFCKTKKGPPLDGTECAPGKWCFKGHCIWKSPEQTYGQDGGWSSWTKFGSCSRSCGGGVRSRSRSCNNPSPAYGGRLCLGPMFEYQVCNSEECPGTYEDFRAQQCAKRNSYYVHQNAKHSWVPYEPDDDAQKCELICQSADTGDVVFMNQVVHDGTRCSYRDPYSVCARGECVPVGCDKEVGSMKADDKCGVCGGDNSHCRTVKGTLGKASKQAGALKLVQIPAGARHIQIEALEKSPHRIVVKNQVTGSFILNPKGKEATSRTFTAMGLEWEDAVEDAKESLKTSGPLPEAIAILALPPTEGGPRSSLAYKYVIHEDLLPLIGSNNVLLEEMDTYEWALKSWAPCSKACGGGIQFTKYGCRRRRDHHMVQRHLCDHKKRPKPIRRRCNQHPCSQPVWVTEEWGACSRSCGKLGVQTRGIQCLLPLSNGTHKVMPAKACAGDRPEARRPCLRVPCPAQWRLGAWSQCSATCGEGIQQRQVVCRTNANSLGHCEGDRPDTVQVCSLPACGGNHQNSTVRADVWELGTPEGQWVPQSEPLHPINKISSTEPCTGDRSVFCQMEVLDRYCSIPGYHRLCCVSCIKKASGPNPGPDPGPTSLPPFSTPGSPLPGPQDPADAAEPPGKPTGSEDHQHGRATQLPGALDTSSPGTQHPFAPETPIPGASWSISPTTPGGLPWGWTQTPTPVPEDKGQPGEDLRHPGTSLPAASPVT

SEQ ID NO: 4 - protein, 1226 aa, synthetic construct
  REGION 1..1226: note = Human ADAMTS14 isoform D (UniProtKB: Q8WXS8-4)
MAPLRALLSYLLPLHCALCAAAGSRTPELHLSGKLSDYGVTVPCSTDFRGRFLSHVVSGPAAASAGSMVVDTPPTLPRHSSHLRVARSPLHPGGTLWPGRVGRHSLYFNVTVFGKELHLRLRPNRRLVVPGSSVEWQEDFRELFRQPLRQECVYTGGVTGMPGAAVAISNCDGLAGLIRTDSTDFFIEPLERGQQEKEASGRTHVVYRREAVQQEWAEPDGDLHNEAFGLGDLPNLLGLVGDQLGDTERKRRHAKPGSYSIEVLLVVDDSVVRFHGKEHVQNYVLTLMNIVDEIYHDESLGVHINIALVRLIMVGYRQSLSLIERGNPSRSLEQVCRWAHSQQRQDPSHAEHHDHVVFLTRQDFGPSGMQGYAPVTGMCHPLRSCALNHEDGFSSAFVIAHETGHVLGMEHDGQGNGCADETSLGSVMAPLVQAAFHRFHWSRCSKLELSRYLPSYDCLLDDPFDPAWPQPPELPGINYSMDEQCRFDFGSGYQTCLAFRTFEPCKQLWCSHPDNPYFCKTKKGPPLDGTECAPGKWCFKGHCIWKSPEQTYGQDGGWSSWTKFGSCSRSCGGGVRSRSRSCNNPSPAYGGRLCLGPMFEYQVCNSEECPGTYEDFRAQQCAKRNSYYVHQNAKHSWVPYEPDDDAQKCELICQSADTGDVVFMNQVVHDGTRCSYRDPYSVCARGECVPVGCDKEVGSMKADDKCGVCGGDNSHCRTVKGTLGKASKQAGALKLVQIPAGARHIQIEALEKSPHRIVVKNQVTGSFILNPKGKEATSRTFTAMGLEWEDAVEDAKESLKTSGPLPEAIAILALPPTEGGPRSSLAYKYVIHEDLLPLIGSNNVLLEEMDTYEWALKSWAPCSKACGGGIQFTKYGCRRRRDHHMVQRHLCDHKKRPKPIRRRCNQHPCSQPVWVTEEWGACSRSCGKLGVQTRGIQCLLPLSNGTHKVMPAKACAGDRPEARRPCLRVPCPAQWRLGAWSQCSATCGEGIQQRQVVCRTNANSLGHCEGDRPDTVQVCSLPACGGNHQNSTVRADVWELGTPEGQWVPQSEPLHPINKISSTEPCTGDRSVFCQMEVLDRYCSIPGYHRLCCVSCIKKASGPNPGPDPGPTSLPPFSTPGSPLPGPQDPADAAEPPGKPTGSEDHQHGRATQLPGALDTSSPGTQHPFAPETPIPGASWSISPTTPGGLPWGWTQTPTPVPEDKGQPGEDLRHPGTSLPAASPVT

SEQ ID NO: 5 - protein, 22 aa, synthetic construct
  REGION 1..22: note = Human ADAMTS14 isoforms A, D signal peptide
MAPLRALLSYLLPLHCALCAAA

SEQ ID NO: 6 - protein, 230 aa, synthetic construct
  REGION 1..230: note = Human ADAMTS14 isoforms A, D propeptide
GSRTPELHLSGKLSDYGVTVPCSTDFRGRFLSHVVSGPAAASAGSMVVDTPPTLPRHSSHLRVARSPLHPGGTLWPGRVGRHSLYFNVTVFGKELHLRLRPNRRLVVPGSSVEWQEDFRELFRQPLRQECVYTGGVTGMPGAAVAISNCDGLAGLIRTDSTDFFIEPLERGQQEKEASGRTHVVYRREAVQQEWAEPDGDLHNEAFGLGDLPNLLGLVGDQLGDTERKRR

SEQ ID NO: 7 - protein, 185 aa, synthetic construct
  REGION 1..185: note = Human ADAMTS14 isoforms B, C propeptide
MVVDTPPTLPRHSSHLRVARSPLHPGGTLWPGRVGRHSLYFNVTVFGKELHLRLRPNRRLVVPGSSVEWQEDFRELFRQPLRQECVYTGGVTGMPGAAVAISNCDGLAGLIRTDSTDFFIEPLERGQQEKEASGRTHVVYRREAVQQEWAEPDGDLHNEAFGLGDLPNLLGLVGDQLGDTERKRR

SEQ ID NO: 8 - protein, 971 aa, synthetic construct
  REGION 1..971: note = Human ADAMTS14 isoforms A, B mature protein
HAKPGSYSIEVLLVVDDSVVRFHGKEHVQNYVLTLMNIVDEIYHDESLGVHINIALVRLIMVGYRQSLSLIERGNPSRSLEQVCRWAHSQQRQDPSHAEHHDHVVFLTRQDFGPSGYAPVTGMCHPLRSCALNHEDGFSSAFVIAHETGHVLGMEHDGQGNGCADETSLGSVMAPLVQAAFHRFHWSRCSKLELSRYLPSYDCLLDDPFDPAWPQPPELPGINYSMDEQCRFDFGSGYQTCLAFRTFEPCKQLWCSHPDNPYFCKTKKGPPLDGTECAPGKWCFKGHCIWKSPEQTYGQDGGWSSWTKFGSCSRSCGGGVRSRSRSCNNPSPAYGGRLCLGPMFEYQVCNSEECPGTYEDFRAQQCAKRNSYYVHQNAKHSWVPYEPDDDAQKCELICQSADTGDVVFMNQVVHDGTRCSYRDPYSVCARGECVPVGCDKEVGSMKADDKCGVCGGDNSHCRTVKGTLGKASKQAGALKLVQIPAGARHIQIEALEKSPHRIVVKNQVTGSFILNPKGKEATSRTFTAMGLEWEDAVEDAKESLKTSGPLPEAIAILALPPTEGGPRSSLAYKYVIHEDLLPLIGSNNVLLEEMDTYEWALKSWAPCSKACGGGIQFTKYGCRRRRDHHMVQRHLCDHKKRPKPIRRRCNQHPCSQPVWVTEEWGACSRSCGKLGVQTRGIQCLLPLSNGTHKVMPAKACAGDRPEARRPCLRVPCPAQWRLGAWSQCSATCGEGIQQRQVVCRTNANSLGHCEGDRPDTVQVCSLPACGGNHQNSTVRADVWELGTPEGQWVPQSEPLHPINKISSTEPCTGDRSVFCQMEVLDRYCSIPGYHRLCCVSCIKKASGPNPGPDPGPTSLPPFSTPGSPLPGPQDPADAAEPPGKPTGSEDHQHGRATQLPGALDTSSPGTQHPFAPETPIPGASWSISPTTPGGLPWGWTQTPTPVPEDKGQPGEDLRHPGTSLPAASPVT

SEQ ID NO: 9 - protein, 974 aa, synthetic construct
  REGION 1..974: note = Human ADAMTS14 isoforms C, D mature protein
HAKPGSYSIEVLLVVDDSVVRFHGKEHVQNYVLTLMNIVDEIYHDESLGVHINIALVRLIMVGYRQSLSLIERGNPSRSLEQVCRWAHSQQRQDPSHAEHHDHVVFLTRQDFGPSGMQGYAPVTGMCHPLRSCALNHEDGFSSAFVIAHETGHVLGMEHDGQGNGCADETSLGSVMAPLVQAAFHRFHWSRCSKLELSRYLPSYDCLLDDPFDPAWPQPPELPGINYSMDEQCRFDFGSGYQTCLAFRTFEPCKQLWCSHPDNPYFCKTKKGPPLDGTECAPGKWCFKGHCIWKSPEQTYGQDGGWSSWTKFGSCSRSCGGGVRSRSRSCNNPSPAYGGRLCLGPMFEYQVCNSEECPGTYEDFRAQQCAKRNSYYVHQNAKHSWVPYEPDDDAQKCELICQSADTGDVVFMNQVVHDGTRCSYRDPYSVCARGECVPVGCDKEVGSMKADDKCGVCGGDNSHCRTVKGTLGKASKQAGALKLVQIPAGARHIQIEALEKSPHRIVVKNQVTGSFILNPKGKEATSRTFTAMGLEWEDAVEDAKESLKTSGPLPEAIAILALPPTEGGPRSSLAYKYVIHEDLLPLIGSNNVLLEEMDTYEWALKSWAPCSKACGGGIQFTKYGCRRRRDHHMVQRHLCDHKKRPKPIRRRCNQHPCSQPVWVTEEWGACSRSCGKLGVQTRGIQCLLPLSNGTHKVMPAKACAGDRPEARRPCLRVPCPAQWRLGAWSQCSATCGEGIQQRQVVCRTNANSLGHCEGDRPDTVQVCSLPACGGNHQNSTVRADVWELGTPEGQWVPQSEPLHPINKISSTEPCTGDRSVFCQMEVLDRYCSIPGYHRLCCVSCIKKASGPNPGPDPGPTSLPPFSTPGSPLPGPQDPADAAEPPGKPTGSEDHQHGRATQLPGALDTSSPGTQHPFAPETPIPGASWSISPTTPGGLPWGWTQTPTPVPEDKGQPGEDLRHPGTSLPAASPVT

SEQ ID NO: 10 - protein, 202 aa, synthetic construct
  REGION 1..202: note = Human ADAMTS14 isoforms A, B peptidase M12B domain
YSIEVLLVVDDSVVRFHGKEHVQNYVLTLMNIVDEIYHDESLGVHINIALVRLIMVGYRQSLSLIERGNPSRSLEQVCRWAHSQQRQDPSHAEHHDHVVFLTRQDFGPSGYAPVTGMCHPLRSCALNHEDGFSSAFVIAHETGHVLGMEHDGQGNGCADETSLGSVMAPLVQAAFHRFHWSRCSKLELSRYLPSYDCLLDDP

SEQ ID NO: 11 - protein, 205 aa, synthetic construct
  REGION 1..205: note = Human ADAMTS14 isoforms C, D peptidase M12B domain
YSIEVLLVVDDSVVRFHGKEHVQNYVLTLMNIVDEIYHDESLGVHINIALVRLIMVGYRQSLSLIERGNPSRSLEQVCRWAHSQQRQDPSHAEHHDHVVFLTRQDFGPSGMQGYAPVTGMCHPLRSCALNHEDGFSSAFVIAHETGHVLGMEHDGQGNGCADETSLGSVMAPLVQAAFHRFHWSRCSKLELSRYLPSYDCLLDDP

SEQ ID NO: 12 - protein, 91 aa, synthetic construct
  REGION 1..91: note = Human ADAMTS14 disintegrin domain
FDPAWPQPPELPGINYSMDEQCRFDFGSGYQTCLAFRTFEPCKQLWCSHPDNPYFCKTKKGPPLDGTECAPGKWCFKGHCIWKSPEQTYGQ

SEQ ID NO: 13 - protein, 56 aa, synthetic construct
  REGION 1..56: note = Human ADAMTS14 TSP type 1 repeat 1
DGGWSSWTKFGSCSRSCGGGVRSRSRSCNNPSPAYGGRLCLGPMFEYQVCNSEECP

SEQ ID NO: 14 - protein, 61 aa, synthetic construct
  REGION 1..61: note = Human ADAMTS14 TSP type 1 repeat 2
DTYEWALKSWAPCSKACGGGIQFTKYGCRRRRDHHMVQRHLCDHKKRPKPIRRRCNQHPCS

SEQ ID NO: 15 - protein, 60 aa, synthetic construct
  REGION 1..60: note = Human ADAMTS14 TSP type 1 repeat 3
QPVWVTEEWGACSRSCGKLGVQTRGIQCLLPLSNGTHKVMPAKACAGDRPEARRPCLRVP

SEQ ID NO: 16 - protein, 55 aa, synthetic construct
  REGION 1..55: note = Human ADAMTS14 TSP type 1 repeat 4
CPAQWRLGAWSQCSATCGEGIQQRQVVCRTNANSLGHCEGDRPDTVQVCSLPACG

SEQ ID NO: 17 - protein, 117 aa, synthetic construct
  REGION 1..117: note = Human ADAMTS14 spacer domain
LKLVQIPAGARHIQIEALEKSPHRIVVKNQVTGSFILNPKGKEATSRTFTAMGLEWEDAVEDAKESLKTSGPLPEAIAILALPPTEGGPRSSLAYKYVIHEDLLPLIGSNNVLLEEM

SEQ ID NO: 18 - protein, 39 aa, synthetic construct
  REGION 1..39: note = Human ADAMTS14 PLAC domain
STEPCTGDRSVFCQMEVLDRYCSIPGYHRLCCVSCIKKA

SEQ ID NO: 19 - protein, 124 aa, synthetic construct
  REGION 1..124: note = Human ADAMTS14 proline-rich region
PNPGPDPGPTSLPPFSTPGSPLPGPQDPADAAEPPGKPTGSEDHQHGRATQLPGALDTSSPGTQHPFAPETPIPGASWSISPTTPGGLPWGWTQTPTPVPEDKGQPGEDLRHPGTSLPAASPVT